SEQUENCE LISTING (12 sequences)

SEQ ID NO: 1 - DNA, 20 bp, Artificial Sequence
  XBP-Specific Forward Primer for RT PCR
ccttgtagttgagaaccagg

SEQ ID NO: 2 - DNA, 20 bp, Artificial Sequence
  XBP-specific reverse primer for RT-PCR
ggggcttggtatatatgtgg

SEQ ID NO: 3 - DNA, 21 bp, Artificial Sequence
  IREl  alpha primer for RNAi screening
cagcacggacgtcaagtttga

SEQ ID NO: 4 - DNA, 21 bp, Artificial Sequence
  ATF6 alpha primer for RNAi screening
cagcaaccaattatcagttta

SEQ ID NO: 5 - DNA, 25 bp, Artificial Sequence
  Derlin-1 primer for RNAi screening
tcccggcgatcacgcgctattggtt

SEQ ID NO: 6 - DNA, 19 bp, Artificial Sequence
  Caspase 2 primer for RNAi screening
gcaccatgcctttgagctt

SEQ ID NO: 7 - DNA, 25 bp, Artificial Sequence
  IREl  alpha primer - targeted 1
ccctacctacacggtggacatcttt

SEQ ID NO: 8 - DNA, 25 bp, Artificial Sequence
  IREl  alpha primer - targeted 2
gacctgcgtaaattcaggacctata

SEQ ID NO: 9 - DNA, 39 bp, Artificial Sequence
  PCR primer - mouse ATF6 forward
ggtaccgcgggcgcgccatggagtcgccttttactccgg

SEQ ID NO: 10 - DNA, 36 bp, Artificial Sequence
  PCR primer - mouse ATF6 Rev
cttggatccgcggcctactgcaacgactcagggatg

SEQ ID NO: 11 - DNA, 20 bp, Artificial Sequence
  PCR XBP1 primer - For
ccttgtagttgagaaccagg

SEQ ID NO: 12 - DNA, 20 bp, Artificial Sequence
  PCR XBP1 primer - Rev
ggggcttggtatatatgtgg

(19) United States
(12) Patent Application Publication
(10) Pub. No.: US 2010/0266618 A1
(43) Pub. Date: Oct. 21, 2010

(54) COMPOSITIONS AND METHODS FOR AUGMENTING ACTIVITY OF ONCOLYTIC VIRUSES

(75) Inventors: David Stojdl, Ottawa (CA); Douglas Mahoney, Ottawa (CA)

Correspondence Address: DAVID S. RESNICK, NIXON PEABODY LLP, 100 SUMMER STREET, BOSTON, MA, 02110-2131, US

(73) Assignee: CHILDREN'S HOSPITAL OF EASTERN ONTARIO RESEARCH INSTITUTE, OTTAWA (CA)

(21) Appl. No.: 12/726,928

(22) Filed: Mar. 18, 2010

Related U.S. Application Data
(60) Provisional application No. 61/161,297, filed on Mar. 18, 2009.

(51) Int. Cl. A61K 39/395; A61K 35/76; C12Q 1/70; C07H 21/02; C07K 14/00; C07K 16/18; A61K 31/713; A61K 38/16; A61P 35/00

(52) U.S. Cl. 424/174.1; 424/93.6; 424/93.2; 435/5; 536/24.5; 530/350; 530/387.1; 514/44 A; 514/19.3

(57) ABSTRACT

Disclosed are compositions and methods for augmenting activity of oncolytic viruses. Virus activity is augmented by sensitizing cancer or tumour cells through modulation of the Endoplasmic Reticulum (ER) stress response pathway, for instance by introducing into a tumour cell an agent effective to modulate ER stress response and sensitize the tumour cell. The tumour cells are then contacted with an oncolytic virus in an amount effective to reduce viability of the sensitized tumour cell. The oncolytic virus is thereby rendered more effective at lysing or killing the sensitized tumour or cancer cells.

### FIELD OF INVENTION

The present invention relates to compositions and methods for augmenting activity of oncolytic viruses. In particular, oncolytic virus activity is augmented by sensitizing cancer or tumour cells through modulation of the Endoplasmic Reticulum (ER) stress response pathway.

### BACKGROUND OF THE INVENTION

Despite major advances in the understanding of cancer over the last 50 years, it remains one of the most important health challenges worldwide. Innovative approaches are needed to complement current drug based therapeutic strategies, and oncolytic viruses represent one such promising tool in the fight against cancer.

Oncolytic viruses preferentially infect and lyse cancer cells. They have been shown to act: (i) by directly destroying tumour cells via their inherent cytolytic activity, and (ii) through modification to function as vectors for delivering genes expressing anticancer proteins to a tumour site.

One example of an oncolytic virus having cytolytic activity is ONYX-015. ONYX-015 is the commercial name of an adenovirus mutant (dll 520) that is replication-restricted in normal cells having a wild-type p53 gene. ONYX-015 has been shown to replicate and kill tumour cells lacking a functional p53.

When used as a vector, therapeutic or cytotoxic genes can be delivered by the oncolytic virus to a tumour site where the products of these genes either directly or indirectly inhibit tumour growth. A number of different genes have been used for such applications, including human cytokine genes, tumour suppressor genes, bacterial or viral prodrug-activating enzyme encoding genes (suicide genes) and genes which make the tumour mass more susceptible to radiation and chemotherapy.

A variety of different virus strains have been studied, including naturally occurring or genetically modified versions of adenovirus, herpes simplex virus (“HSV”), reovirus, poxvirus (e.g. vaccinia virus and Myxoma virus), vesicular stomatitis virus (“VSV”), poliovirus, Newcastle disease virus (“NDV”), and measles. However, such viruses often lack sufficient potency as monotherapies to be completely clinically effective anticancer agents.

In an effort to improve clinical efficacy, candidate viruses have been genetically engineered to express therapeutic transgenes, and have been combined with other common oncolytic therapies. While such studies are ongoing with encouraging success, there continues to be a need for ways to enhance potency and efficacy, and generally make oncolytic viruses more effective cancer therapeutics.

### SUMMARY OF THE INVENTION

It is an object of the invention to provide improved compositions and methods for augmenting activity of oncolytic viruses.

The invention relates to a method of reducing viability of a tumor cell in a subject, comprising the steps of: (i) introducing into a tumor cell in the subject an agent effective to modulate endoplasmic reticulum (ER) stress response and sensitize the tumour cell to cytolytic activity of an oncolytic virus in the subject; and (ii) contacting the tumor cell with an oncolytic virus in an amount effective to reduce viability of the sensitized tumour cell, wherein viability of the tumor cell is reduced by the oncolytic virus. In a preferred embodiment, the oncolytic virus lyses or kills the sensitized tumour cell.

In a further embodiment, the invention relates to a method of modulating sensitivity of cancer cells to infection by an oncolytic virus, comprising introducing into a cancer cell an agent effective to modulate endoplasmic reticulum (ER) stress response and sensitize the cancer cell to cytolytic activity of the oncolytic virus, wherein the cancer cells are sensitized to infection by the oncolytic virus.

According to the methods described herein, the agent may be effective to enhance, diminish or inhibit the ER stress response in said subject. In one preferred embodiment, the agent is effective to inhibit the ER stress response in said subject. In a further embodiment, the agent may be a siRNA specific to ERN, ATF6, Derlin1, Derlin2 or SEC61. Alternately, the agent may be a molecule effective to modulate ERN, ATF6, Derlin1, Derlin2 or SEC61 signaling, for instance a molecule effective to block or enhance ERN, ATF6, Derlin1, Derlin2 or SEC61 signaling. It is also contemplated that the agent may be a modified oncolytic virus wherein the modification renders the oncolytic virus effective to modulate ER stress response and sensitize the tumour cell to cytolytic activity.

It is further envisioned that the tumour cells or cancer cells described herein may be any tumour or cancer cell that is susceptible to oncolytic virus infection and modulation of the ER stress response. Without wishing to be limiting in any way, such cancer and tumour cells may be colon cancer cells, lung cancer cells, liver cancer cells, prostate cancer cells, bladder cancer cells, neck and mouth cancer cells, breast cancer cells, glioblastoma cells, lymphoma cells, carcinoma cells, renal cell cancer cells, pancreatic cancer cells, ovarian cancer cells and any other such cancer or tumour cells known in the art.

In further embodiments, the oncolytic virus may be any oncolytic virus, such as but without wishing to be limiting, a native or modified herpes virus, Adenovirus, Adeno-associated virus, influenza virus, reovirus, rhabdovirus, Newcastle virus, vaccinia virus, poliovirus, measles virus, mumps virus, sindbis virus (SIN) or sendai virus (SV). In preferred embodiments the oncolytic virus may be a native or modified rhabdovirus, for example a native or modified vesicular stomatitis virus (VSV) or Maraba virus. By ‘modified’, it is meant that the virus is a mutant virus modified with a function-improving mutation to make the virus a more effective cancer or tumour cell lysing agent.

The invention further relates to a method of identifying a tumour cell sensitizing agent effective for sensitizing tumour cells to infection by an oncolytic virus. The method comprises: (i) providing a test molecule with putative endoplasmic reticulum (ER) stress response modulating activity, (ii) adding the test molecule to a sample of said tumor cells, (iii) contacting the tumor cells with the oncolytic virus, and (iv) comparing cytolytic activity of the oncolytic virus in the sample of tumour cells with the test molecule to activity of the oncolytic virus in a sample of tumour cells without the test molecule, wherein increased cytolytic activity of the oncolytic virus in the sample of tumour cells with the test molecule indicates the presence of a tumour cell sensitizing agent.

The test molecule described above may be any molecule suspected of having ER stress response modulating activity. Such molecules may be a small molecule, a protein, a nucleic acid, an antibody, or any other non-limiting example of a putative test molecule.

The invention further provides compounds effective to modulate endoplasmic reticulum (ER) stress response and sensitize a tumour cell to cytolytic activity of an oncolytic virus in a subject. Such compounds may be effective to inhibit the ER stress response in the subject. For instance, the compound may be a siRNA specific to ERN, ATF6, Derlin1, Derlin2 or SEC61, or a molecule effective to modulate ERN, ATF6, Derlin1, Derlin2 or SEC61 signaling. In another embodiment, the compounds may augment the ER stress response in the tumour to improve oncolytic therapy.

In an embodiment, the compound is effective to block or enhance ERN, ATF6, Derlin1, Derlin2 or SEC61 signaling. In a further embodiment, the compound is a protein, a small molecule, a nucleic acid, or an antibody.

The above-described compounds may also be formulated into a composition, for instance a pharmaceutical composition including a pharmaceutically acceptable carrier or excipient.

Also contemplated by the present invention is a modified oncolytic virus, wherein the modification renders the oncolytic virus effective to modulate endoplasmic reticulum (ER) stress response and sensitize a tumour cell to cytolytic activity of the oncolytic virus in a subject. The modified oncolytic virus may be effective to inhibit the ER stress response in the subject. In another embodiment, the modified oncolytic virus may augment the ER stress response in the tumour to improve oncolytic therapy.

In an embodiment, the oncolytic virus may be modified to include a nucleotide specific to ERN, ATF6, Derlin1, Derlin2 or SEC61 or a nucleotide encoding a molecule effective to modulate ERN, ATF6, Derlin1, Derlin2 or SEC61 signaling. In a further embodiment, the modified oncolytic virus may be effective to block or enhance ERN, ATF6, Derlin1, Derlin2 or SEC61 signaling. In a further embodiment, the molecule may be a protein or an antibody.

The above-described modified oncolytic virus may also be formulated into a composition, for instance a pharmaceutical composition including a pharmaceutically acceptable carrier or excipient.

The invention also relates to a method of sensitizing a tumor to cytolytic activity of an oncolytic virus, said method comprising inducing in a subject a mild stress to the endoplasmic reticulum (ER).

In a non-limiting embodiment, inducing the mild stress may comprise genetically disrupting an ER stress response gene, for instance a ER stress response gene such as IRE1/ERN, DERLIN, and ATF6. In another non-limiting embodiment, inducing the mild stress may comprise chemically inhibiting IRE1/ERN1. For instance, compound 2 (described herein) is administered to the subject to chemically inhibit IRE1/ERN1. In a further non-limiting embodiment, inducing the mild stress may comprise chemically inhibiting cyclophilins which blocks the function of chaperones in the ER. Without wishing to be limiting, Cyclosporin A can be administered to chemically inhibiting the cyclophilins. In another non-limiting embodiment, inducing the mild stress may comprise chemically inhibiting protein glycosylation and producing more unfolded proteins in the ER. For instance, but without wishing to be limiting, Tunicamycin can be administered to chemically inhibit protein glycosylation.

It is also to be understood that the above-described compound can be effective to induce an ER stress and render tumour cells susceptible to a virus infection. In addition, yet without wishing to be limiting, the compound can be effective to initiate caspase 2 mediated cell death in response to a virus infection, and render tumour cells susceptible to a virus infection.

In certain non-limiting embodiments, the compound may be one of the following

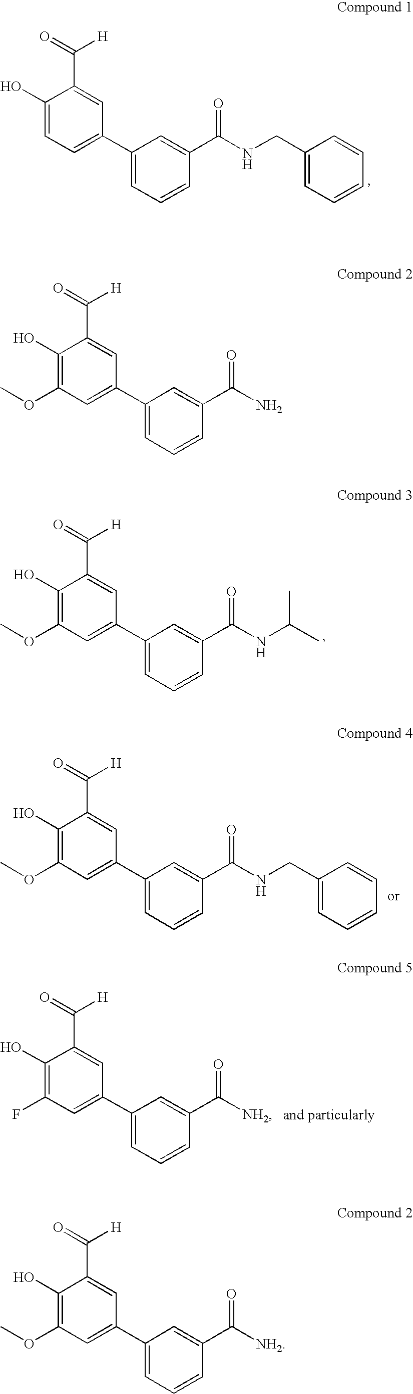

### BRIEF DESCRIPTION OF THE DRAWINGS

These and other features of the invention will become more apparent from the following description in which reference is made to the appended drawings wherein:

FIG. 1 is a schematic describing the functional genetic screen used to identify host genes that modulate oncolytic virus therapy. Briefly, cells in 384 well format were reverse transfected with 10 nM siRNA pools from an arrayed genome wide library of siRNA (Dharmacon Inc. USA). After an incubation of 72 hours, cells were infected with Maraba virus at an MOI of 0.05 and further incubated for 48 hours. Cell viability was measured using Alamar Blue™ vital dye assay. Alternatively cells were washed, fixed and stained with hoechst nuclear stain and scored for cell number and nuclear morphology to assay cell viability;

FIG. 2 shows a summary of the genome-wide RNAi screen in two cancer cell lines: (A) Venn diagram indicating the number of genes scored as “hits” in the screen for each cell line. Hits are designated as genes with viability scores greater than two standard deviations of the negative control from the mean of the overall screen. In addition, genes with viability scores of one standard deviation of the negative control from the mean of RNAi alone screen were filtered from the list to remove false positives due to the effects of RNAi alone. (B) Bioinformatic analysis of the 485 hits common to both cell lines;

FIG. 3 illustrates results showing IRE1/ERN knockdown sensitizing of cancer cells to Maraba virus killing: (A) Cell lines were transfected with siRNA targeting IRE1alpha (ERN1) or IRE1beta (ERN2) or control in 96 well format. After 72 hours, wells were infected with Maraba virus at various MOIs. Viability was monitored by Alamar Blue™ viability assay following 48 h of infection. Experiments were performed in triplicate and plotted as the mean, with error bars representing the standard error. EC50 values were calculated for each condition and compared to the control to determine fold sensitization. (B) Summary of the results from (A) indicating the effects of loss of ERN mRNA on Maraba virus killing of tumour lines. (C) Western blot confirming the inhibition of ERN-1 (IRE1alpha) protein expression; and

FIG. 4 illustrates results showing Maraba virus activates the Unfolded Protein Response: (A) U373 and OVCAR-8 cells were treated with tunicamycin (5 μg/mL) or wild-type Maraba virus (MOI 5) for the indicated amounts of time. Total protein lysates were collected, and immunoblots performed using the indicated antibodies. F-ATF6, Full-length activated transcription factor 6. DG-ATF6, de-glycosylated ATF6. C-ATF-6, cleaved ATF6. P-EIF2α, phosphorylated eukaryotic initiation factor 2α. BIP/GRP78, immunoglobulin heavy chain binding protein/glucose regulated protein 78. XBP(s), spliced X-box binding protein 1. GAPDH, glyceraldehyde dehydrogenase. G, N, P and M indicate Maraba virus proteins. (B) U373 and OVCAR-8 cells were treated with tunicamycin (TM) and Maraba virus, as in (A). Total RNA was extracted and RT-PCR performed using XBP-specific primers. XBP(u), un-spliced XBP. XBP(s), spliced XBP.

FIG. 5 illustrates the results of the genome wide screen of Example 2, and the identification of ER stress response blockade as a potent sensitizer to rhabdovirus-mediated oncolysis. (A) Schematic representation of the screen. (B) Venn diagram outlining the number of overlapping hits, and a table (+=synthetic lethal, −=no interaction) and schematic diagram (hits outlined in red) illustrating key hits within the UPR and ERAD pathways. (C) Phase contrast images of U373 cells treated first with siRNA (72 h) followed by Maraba virus infection (MOI 5; 24 h). (D) Upper panels. Cell viability assays were performed 48 h after Maraba virus infection, which followed 72 h siRNA treatment. Western blots demonstrating protein knockdown are depicted (* denotes non-specific band). Lower panels. Cell viability assays were conducted 48 h after virus infection, in U373 cells ectopically expressing mouse ATF6α (or GFP control)±siRNA targeting human ATF6α (or NT control). (E) Representative tumour and normal cell lines were treated with siRNA targeting IRE1α for 72 h followed by Maraba virus (MOI 0.1). Cell viability assays were performed 48 h later. (F) Cell viability assays were performed on a panel of cancer-derived cell lines 48 h after Maraba virus infection (MOI 5), which followed UPR knockdown (72 h). Data is represented as “log sensitization”, which is defined as the reduction in the amount of Maraba virus (on a log₁₀ scale) required to obtain an EC₅₀. The degree of functionality of the interferon system is also plotted, with (−) indicating completely defective and (+++) indicating completely functional (N/A indicates cell lines not analyzed). (G) Cell viability was measured after 48 h infection with wild type or “double mutant” Maraba virus of wild type VSV (MOI 0.1), which followed 72 h siRNA treatment (*=p<0.05; #=p<0.01).

FIG. 6 illustrates that UPR knockdown sensitizes U373 cells towards Maraba virus mediated killing. U373 cells were treated with non- or IRE1α-targeting RNAi duplexes for 72 h prior to Maraba-WT infection. After 48 h, cell viability was assessed using Resazurin sodium salt (*=p<0.05).

FIG. 7 illustrates that acute inhibition of the ER stress response is not sufficient to sensitize tumour cells to viral oncolysis. (A) U373 cells were treated with the ER stressor tunicamycin (5 μg/mL) or Maraba virus (MOI 5). Total RNA was collected and RT-PCR for XBP1 splicing performed. (B) Cells were treated as in (A). Total cell lysates were collected and immunoblot analyses conducted (DG-ATG6α=deglycosylated ATF6α, due to the inhibitory effect of TM on glycosylation). (C) U373 cells were treated with putative IRE1α small molecule inhibitors (2 h) prior to tunicamycin treatment (4 h). Total RNA was collected and RT-PCR performed. RNAi targeting IRE1α (72 h) was used as a control. (D) U373 cells were treated with Compound 2 (50 uM) or controls for 4 or 48 h prior to Maraba virus infection. Cell viability assays were performed 48 h later. (E) Cells were treated as in (D) and combination index analyses performed. (F) U373 cells were treated with DMSO or cyclosporine (25 μM) for 4 or 48 h before Maraba virus infection. Cell viability was assessed 48 h later (*=p<0.05).

FIG. 8 shows ER preload rewires cancer cells for caspase 2-mediated apoptosis in response to oncolytic virus infection. (A) Cells were treated with siRNA for 0-72 h. Total cell lysates were collected followed by immunoblot analyses. (B) Cells were treated with siRNA (72 h), followed by infection with Maraba virus (MOI 5). Total cell lysates were collected and immunoblots performed. (C) Cells were treated as in (B) or treated with tunicamycin (5 ug/mL) for 24 h followed by 24 h “washout”, after which cells were infected with Maraba virus (MOI 5). Total cell lysates were collected at the indicated timepoints and immunoblots performed. (D-E) Cells were treated as in (B), and Western blots performed. (F) Schematic diagram depicting ER Preload model. Rhabdovirus infection of naïve tumour cells (Standard OVT) causes ER stress but fails to trigger a caspase 2 mediated apoptotic response (grayed out dormant response). Inhibiting UPR or ERAD (Combination ER/OV Therapy) induces an ER stress resulting in an adaptive response (rewiring) that predisposes tumour cells to undergo an alternative death program (caspase 2 dependent apoptosis) upon challenge with an oncolytic virus.

FIG. 9 shows UPR knockdown leads to ER preload in U373 glioblastoma cells as compared to GM38 normal human fibroblasts. Cells were treated with siRNA targeting IRE1α (or controls) for 72 h, total cell lysates collected at the indicated timepoints and immunoblots performed.

FIG. 10 shows UPR knockdown has no bearing on virus infectivity. (A) U373 cells were treated with siRNA (72 h) prior to Maraba infection (MOI 5). Total cell lysates were taken at the indicated timepoints post-infection and Western blots performed. (B) Cells were treated as in (A), and single-step growth analyses conducted. (C) Cells were treated as in (A), and phase contrast and fluorescent microscopy images captured following infection.

FIG. 11 shows Maraba virus infection following UPR knockdown leads to Caspase-2 activation in U373 glioblastoma cells but not GM38 normal human fibroblasts. Cells were treated with siRNA targeting IRE1α (or controls) for 72 h, total cell lysates collected at the indicated timepoints and immunoblots performed.

FIG. 12 shows a pharmacokinetic analysis of Compound 2 in CD1 nude mice. (A) CD-1 nude mice (n=3/group) were given a single dose of Compound 2 (500, 125, or 25 mg/kg, IP) and blood was drawn from the saphenous vein at 30 or 120 min. The plasma [Compound 2] was determined by LC-MS, and regression analysis conducted. (B) CD-1 nude mice (n=3/group) were given a single dose of Compound 2 (50 mg/kg, IV or 250 mg/kg, IP) and blood was drawn from the saphenous vein at 0, 5, 15, 30, 60, 120, 240, 360, 480, and 1440 min. Plasma was analyzed for [Compound 2] by LC-MS. The dotted line indicates [20 μM], which was the lowest, maximally effective dose in cell culture experiments. (C) Pharmacokinetic parameters, determined from data in (B), depicting maximum concentrations (Cmax), time at maximum concentration (Tmax), half life T_{1/2}), area under the curves (AUC), clearance rate (CL), volume of distribution (Vc), and relative bioavailability (F=(AUC PO/AUC IV)*100).

FIG. 13 shows chemical inhibition of IRE1α potentiates oncolytic therapy in vivo. (A) Luciferase-tagged OVCAR-4 cells (5e⁶) were delivered intraperitoneally (IP) into CD-1 nude mice. At day 14, mice were treated twice daily with Compound 2 (250 mg/Kg; IP delivery) or vehicle for 6 consecutive days (drug TX window is outlined by the dotted lines). Maraba-DM treatment (1e⁵ PFU; IV injections) was initiated 48-72 h later (virus injections depicted by arrows). Tumours were regularly evaluated using IVIS bioluminescent imaging. The graph depicts relative change in luminescent signal, which corresponds to tumour size. (B) Representative bioluminescent images from (A). (C) EMT6 cells were treated with Compound 2 (50 uM) or vehicle for 48 h prior to Maraba virus infection. Viability assays were conducted 48 h later. (D) Luciferase-tagged EMT6 cells (1e⁵) were implanted into the breast fat pads of Balb/c mice. Compound 2 treatment was initiated at Day 7 (250 mg/Kg; IP; twice daily for 6 days; treatment window depicted by the dotted box). Maraba virus injections (1e⁷ PFU; IV) commenced on Day 10 (black arrows). Bioluminescent data is plotted (as above). (E) Kaplan-meier curve depicting mouse survival in an EMT6 model. The experiment was done as in (D), except that Compound 2 treatment was extended for an additional six days. (*=p<0.05).

### DETAILED DESCRIPTION

Disclosed herein are improved compositions and methods for augmenting activity of oncolytic viruses, which are obtained through the manipulation of the endoplasmic reticulum (ER) stress response. As will be described in greater detail in the following, modulators of the ER stress response pathway can sensitize tumour or cancer cells to the cytolytic activity of an oncolytic virus.

A wide range of viruses are contemplated as oncolytic viruses in the present invention, such as but not limited to herpes viruses, Adenovirus, Adeno-associated virus, influenza virus, reovirus, rhabdoviruses such as vesicular stomatitis virus (VSV) and Maraba virus, Newcastle virus, vaccinia virus, poliovirus, measles virus, mumps virus, sindbis virus (SIN) and sendai virus (SV).

Oncolytic viruses may additionally or alternatively be targeted to specific tissues or tumor tissues. This can be achieved for example through transcriptional targeting of viral genes (e.g. WO 96/39841) or through modification of viral proteins that are involved in the cellular binding and uptake mechanisms during the infection process (e.g. WO 2004033639 or WO 2003068809).

### Experiments

### Experiment 1:

### ER Stress Response

Increased levels of unfolded proteins in the endoplasmic reticulum (ER) of all eukaryotes trigger the unfolded protein response (UPR). Several cellular pathways are involved in mitigating this stress. The ER stress pathway is responsible for dealing with unfolded protein load within the endoplasmic reticulum (reviewed in Kincaid et al., 2007, Antioxid Redox Signal, 9(12):2373-87).

Yeast have a single response to dealing with unfolded proteins through a protein kinase called IRE1. This protein kinase is activated in response to accumulated unfolded proteins within the ER and through its endoribonuclease activity, catalyses the noncanonical splicing of xbp1 mRNA to code for a functional transcription factor upregulating the expression of genes required to ameliorate the stress.

Mammalian cells also make use of the archetypal IRE1 signalling cascade in response to ER stress, but have evolved another parallel response through the ATF6 transcription factor. In unstressed cells, ATF6 resides in a transmembrane protein spanning the ER membrane. Following a stimulation of unfolded proteins in the lumen of the ER, a key at six trends locates that Golgi apparatus whereupon it is cleaved by a resident peptidase forming a soluble transcription factor responsible for the up-regulation of a subset of stress response genes. In addition to this pathway mammalian cells also possess a translational attenuation response. When unfolded proteins accumulate in the lumen of the ER, a protein kinase called PERK phosphorylates elF2alpha in the cytoplasm and attenuates global translation. This response serves to decrease ER load by stemming the influx of proteins into the ER by blocking protein production. A final response to ER stress is the selective translocation of terminally unfolded proteins from the ER into the cytoplasm for proteosome-mediated degradation. This response is termed the ER-associated Degradation (ERAD) response.

### Cancer and ER Stress

Mounting evidence demonstrates that during the etiology of a tumour, cancer cells undergo sustained and/or transient ER stress. Mutations give rise to protein species with suboptimal folding, and hypoxia impedes proper folding of proteins within the ER. Several components of the ER stress response pathway are upregulated in a variety of cancers (Shuda et al., 2003, J. Hepatol. 2003, 38(5):605-14). Manipulating ER stress sensitizes cancer cells to hypoxia (Bi et al., 2005, EMBO J., 24:3470-81) and to chemotherapeutics (Nawrocki et al., 2005, Cancer Res. 65:11658-66).

### Viruses and ER Stress

Viruses induce ER stress, for example: HCV (Joyce et al., 2009, PLoS Pathog., 5(2):e1000291), SARS (Ye et al., 2008, Biochim Biophys Acta.,1780:1383-7), West Nile (Medigeshi et al., 2007, J. Virol., 81:10849-60), Hepatitis B (Li et al., 2007, Virus Res., 124:44-9), Hantavirus, Japanese Encephalitis virus (Su et al., 2002, J. Virol., 76:4162-71), RSV (Bitko et al., 2001, J. Cell Biochem., 80:441-54), influenza (Watowich et al., 1991, J. Virol., 65:3590-7), Herpes (Lee 2008), and dengue fever virus (Umareddy et al., 2007, Virol J., 4:91). Some viruses have the ability to manipulate the cell's response to ER stress: (Bechill et al., 2008, J. Virol.; 82:4492-501; Isler et al., 2005, J. Virol., 79:6890-9; Tardif et al., 2004, J. Biol Chem., 279:17158-64).

Replicating virus based therapeutics, or oncolytic viruses, are a rapidly emerging and promising treatment modality for a wide range of cancers. In pre-clinical studies, oncolytic viruses have produced remarkable results in a variety of experimental animal models including human xenografts in nude mice and syngeneic animal tumours (see Hawkins et al., 2002, Lancet Oncol. 3:17-26; and VähäKoskela 2007 for review). Successfully tested oncolytic viruses include: vesicular stomatitis virus (VSV) (Stojdl et al., 2003, Cancer Cell., 4:263-75; Lun et al., 2006, J. Natl. Cancer Inst., 98:1546-57), adenovirus (AdV) (Ries et al., 2002, Br. J. Cancer, 86:5-11), reovirus (Coffey et al., 1998, Science, 282:1332-4), Newcastle disease virus (NDV) (Lorene et al., 1994, J. Natl. Cancer Inst., 86:1228-33; and Schirrmacher et al., 2001, Int. J. Oncol., 18:945-52), herpes simplex virus (HSV) (Todo et al., 1999, Hum. Gene Ther., 10:2741-55) and vaccinia virus (McCart et al., 2001, Cancer Res., 61:8751-7). Several of these viruses have been, and are continuing to be tested in human clinical trials; again with encouraging results. For example, in a phase I trial, using a genetically modified herpes virus (HSV G207), patients with malignant gliomas were injected intratumourally and some antitumour efficacy was seen by both radiographic and neuropathologic criteria (Markert et al., 2000, Gene Ther.,7:867-74). Onyx-015, an E1B-55kDa gene-deleted adenovirus has completed two phase II human trials directed at squamous cell carcinomas of the head and neck (Nemunaitis et al., 2001, J. Clin. Oncol., 19:289-98; Lamont et al., 2000, Ann. Surg. Oncol., 2000, 7:588-92). In these studies virus was delivered by a series of intra-tumour injections either in combination with chemotherapy agents (Lamont et al., 2000, Ann. Surg. Oncol., 2000, 7:588-92) or as a single agent (Nemunaitis et al., 2001, J. Clin. Oncol., 19:289-98). Onyx-015 was found to be safe and showed some antitumour activity with 10-30% of patients showing complete responses at injection sites and 30-60% of patients having stabilized disease. In a more recent phase I trial, an engineered form of vaccinia virus demonstrated an excellent safety profile as well as promising efficacy data in 14 patients (Park et al., 2008, Lancet Oncol., 9:533-42). There have been in excess of some 25 clinical trials (mostly phase I) that demonstrate the safety of these virus therapies. Results from more phase 2 and 3 trials are awaited to evaluate the efficacy of these oncolytic viruses.

### Rhabdoviridae:

The Rhabdoviridae viral family is divided into 6 genera, in which the vesicular stomatitis virus (VSV) is one of them. Rhabdoviridae are membrane-enveloped viruses that are widely distributed in nature where they infect vertebrates, invertebrates, and plants. Viral particles contain a helical, nucleocapsid core composed of genomic RNA and protein. Rhabdoviridae have single, negative-strand RNA genomes of 11-12,000 nucleotides. Further information on the Rhabdoviridae family of viruses can be found in Rose and Whitt, 2001, Chapter 38, Rhabdoviridae: The viruses and their replication, in Fields Virology, 4.sup.th edition, pp. 1221-1244, the entirety of which is hereby incorporated by reference.

The inventors have previously shown that VSV has oncolytic properties (Stojdl et al., 2000, Nat. Med., 6:821-5) and have since shown that the VSV M protein antagonizes the innate immune system by blocking nuclear cytoplasmic transport of host mRNA. In doing so, the transcriptional cascade responsible for perpetuating the interferon mediated antiviral program is severed and no IFN is produced from these infected cells (Stojdl et al., 2003, Cancer Cell., 4:263-75). VSV strains with M protein mutations lose their capacity to block the IFN response and were shown to be extremely attenuated in normal cells, yet retain their ability to kill tumour cells (Stojdl et al., 2000, supra; Stojdl et al, 2003, supra). In a variety of subcutaneous, metastatic lung and intraperitoneal mouse models of cancer, systemic injection of the engineered VSV mutants was shown to effectively cure mice of local and disseminated tumours (Stojdl et al., 2000, supra; Stojdl et al. 2003 supra).

Oncolytic virus strains from the rhabdovirus family are described in WO 2009/016433, which is herein incorporated by reference.

### RNAi Technology:

The recent advent of RNAi technology has made it possible to use forward genetics techniques to study the function of mammalian genes (Berns et al., 2004, Nature, 428:431-7; Krishnan et al., 2008, Nature, 455:242-5). This technology is particularly useful for studying host virus interactions as many of the host systems relevant to virus infection are unique to higher order organisms (e.g. interferon signaling).

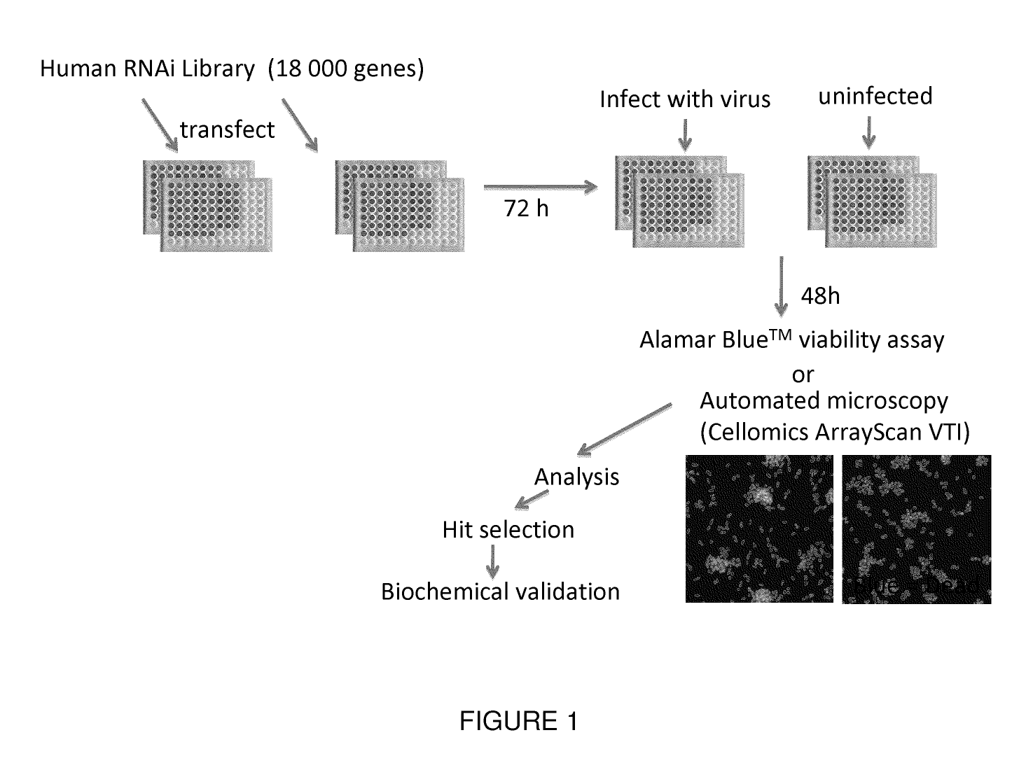

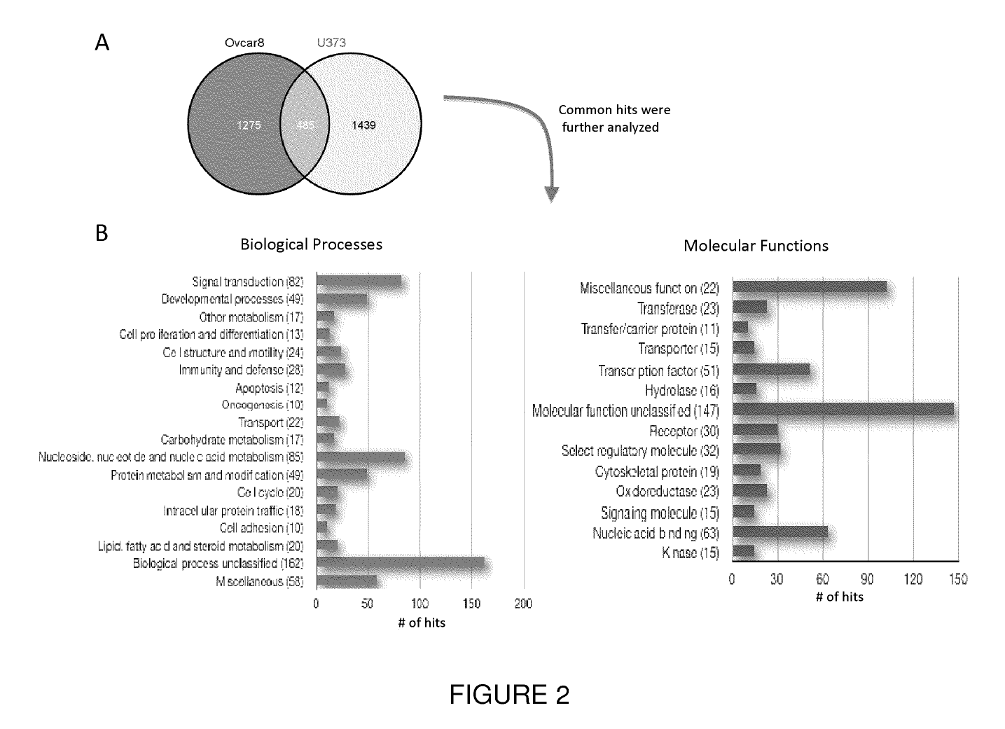

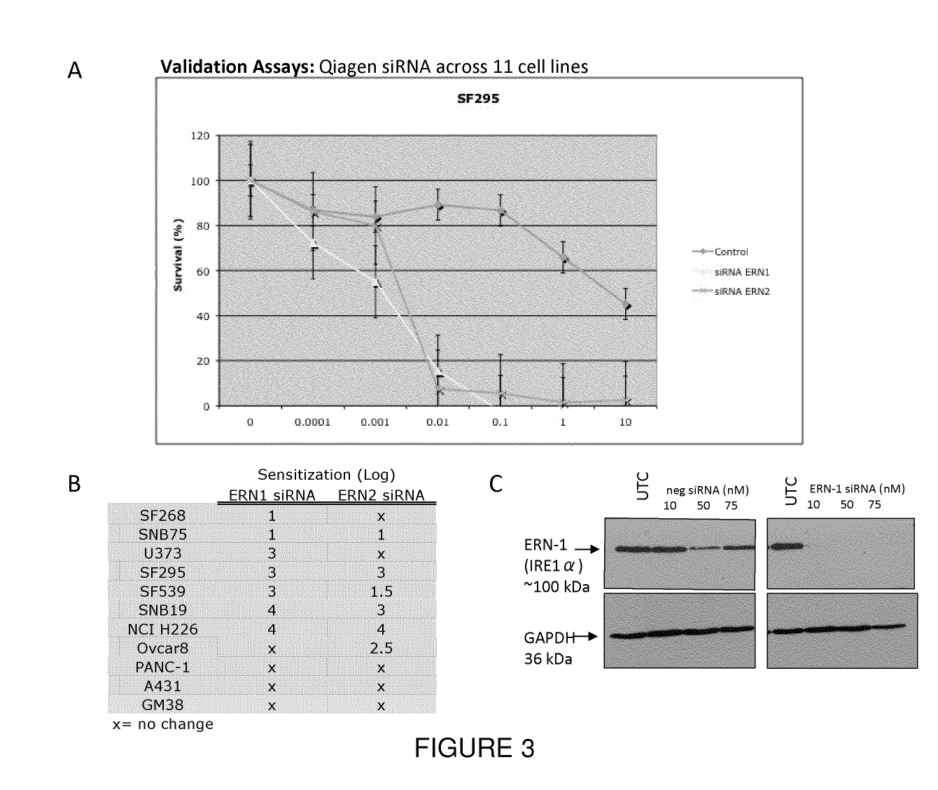

The present inventors have utilized a genome wide RNAi screen to identify host genes, which when neutralized, sensitize cells to a subsequent oncolytic virus infection resulting in increased cell death (FIGS. 1 and 2). This sensitization is specific to cancer cells and does not sensitize normal human primary fibroblasts to oncolytic virus infection (FIG. 3). Accordingly, a means for specifically sensitizing cancer cells to killing by oncolytic virus-based therapy is provided.

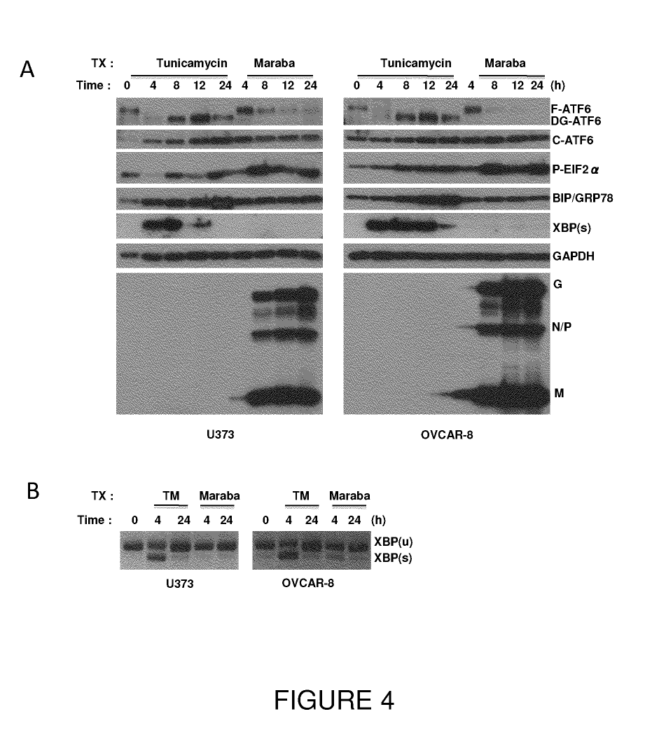

Table 1 lists components of the endoplasmic reticulum (ER) stress pathway that, when removed from the cell or deactivated according to an embodiment of the invention, makes the cell more susceptible to killing by a subsequent infection with an oncolytic virus, for example rhabdovirus-based oncolytic viruses. This is demonstrated by in vitro cytotoxicity assays across a panel of cancer cells using a panel of oncolytic agents as shown in FIGS. 3 and 4.

TABLE 1
Components of the UPR and ERAD pathways identified as synthetic lethal with Maraba
virus infection in both OVCAR 8 and U373 human cancer
cells.

| UPR | | | | ERAD | | | |
|---|---|---|---|---|---|---|---|
| Symbol | GeneID | RefSeq | Synonym | Symbol | GeneID | RefSeq | Synonym |
| ATF6 | 22926 | NM_007348.2 | | DERL1 | 79139 | NM_024295.4 | |
| CREBL1 | 1388 | NM_004381.4 | atf6 beta | DERL2 | 51009 | NM_016041.3 | |
| ERN1 | 2081 | NM_001433.3 | IRE1alpha | sec61a | 29927 | NM_013336.3 | |
| NFYC | 4802 | NM_014223.4 | | sec61g | 23480 | NM_014302.3 | |
| HSPA5BP | 54972 | NM_178031 | | Dnajb9 | 27362 | NM_013760.4 | erdj4 |
| FKBP10 | 60681 | NM_021939 | | DNAJB11 | 51726 | NM_016306.4 | Erdj3 |
| SEP15 | 9403 | NM_004261 | | AMFR | 267 | NM_001144.4 | |

Without wishing to be bound by theory, the enhanced tumour killing capacity is proposed to improve oncolytic virus efficacy by increasing tumour cell death following infection by an oncolytic virus and thereby debulking the tumour more rapidly and requiring less oncolytic virus at the tumour site to achieve similar efficacy.

Tumours are variably and intermittently hypoxic. This is because the vasculature that feeds tumours is often poorly structured. Hypoxia induces ER stress in a number of ways (reviewed in Wouters 2008, supra). It has been proposed that blocking UPR mechanisms would sensitize hypoxic tumour cells to death due to their dependence on these rescue pathways. However, some portions of tumours (often the rims) are not hypoxic as they are fed oxygen from the surrounding healthy stroma, or are adjacent to properly functioning vasculature. These areas of tumours that are not themselves hypoxic would not be affected by ER stress response blocking agents. However, an oncolyic virus infection of these non-hypoxic tumour cells would kill these cells efficiently in combination with ER stress response blockade. Since only tumour cells will be infected by the oncolytic virus, we refer to this as “targeted ER stress”. This combination of oncolytic virus and ER stress response blockade would result in a more complete tumour cell ablation and lessen the chance of re-growth of the tumour; a common problem with current chemotherapy.

Alternatively, it has been shown that oncolytic therapy can induce vascular shutdown and catastrophic hypoxia within tumour cores (Breitbach et al., 2007, Mol. Ther., 15:1686-93). Combination therapy of oncolytic virus with ER stress response blockade would be promoted by the hypoxia induced by the oncolytic virus, even in distant cells not directly infected by the virus. This would again limit the probability that a tumour cell would escape treatment and thereby improve patient outcomes.

Chemical signals (chemokines/cytokines) from the infected cell are released to warn neighboring cells of an imminent virus infection. For example, interferon beta is released from infected cells and induces a paracrine and autocrine signaling cascade that results in a potent antiviral response. Some tumour cells are capable of responding to these chemical signals and mount a defense against an incoming oncolytic virus. It has been demonstrated that interferon type I receptor is downregulated during ER stress. Without wishing to be bound by theory, we propose that inducing an ER stress following oncolytic virus infection will decrease the ability of the infected cell to secrete chemokines/cytokines and as well as the receptors that are required to sense these chemical signals. Further inhibiting of the ER stress response, through combination therapy with a drug or by engineering the oncolytic virus to block this response, should additionally attenuate the chemokine/cytokine mediated antiviral defenses of the infected cell and the surrounding tumour cells. Since the normal healthy cells are themselves resistant to the oncolytic agents, they would not be significantly affected by this mechanism. Therefore, ER stress modulation of innate immunity would specifically sensitize tumour cells to oncolytic virus infection.

### Experimental Design:

A genome wide RNAi screen was conducted to find host genes that could modulate the ability of an oncolytic virus to kill tumour cells. Maraba virus was selected as a representative oncolytic virus from the Rhabdoviridae family. In the following experiments human cancer cells were sensitized to Maraba virus infection by interfering with host cell mRNA expression using siRNA technology. Two cell lines: (1) OVCAR 8 human ovarian carcinoma cells; and (2) U373 human glioblastoma cells, were studied as representative unrelated malignancies in an effort to identify genes that were common to many cancers, and not necessarily specific to one indication. Genes were identified that, when inhibited or augmented, gave rise to improvements in oncolytic activity.

“Hits” from the screens were analyzed for their known functions and it was determined that several of these genes were components of the host ER stress response pathways. Specifically, IRE1 and ATF6alpha and ATF6beta were identified as components of the UPR. IRE1 is known to activate the transcription factor XBP1 through a non-canonical mRNA splicing mechanism in the cytoplasm. Interestingly, the transcriptional co-activator NFYC known to bind and cooperate with both ATF6 and XBP1 was also identified as a “hit”. In addition, components of the ER activated Degradation (ERAD) pathway were identified in the primary screen. AMFR and DERL are known to form a complex and are responsible for tagging and removing terminally unfolded protein from the ER for proteosomal degradation.

These results strongly indicated that modulating ER stress responses through multiple pathways all sensitized cells to Maraba virus infection induced cell death.

### Experimental Procedures:

Genome-wide Screening Procedure: Cells were reverse transfected in 384 well format using 10 nM of Dharmacon siGenome SmartPool human set (Invitrogen USA). For OVCAR 8 human ovarian carcinoma cells 2500 cells/well were transfected using RNAiMax (Invitrogen, USA) (0.05 ul/well) in a total volume of 40 ul of DMEM containing 10% FBS. Alternatively U373 human glioblastoma cells were similarly reverse transfected using Oligofectamine (0.05 ul/well) at a density of 625 cell/well. Plates were incubated for 72 hours to allow for siRNA mediated mRNA down modulation at which time plates were either mock infected or infected with recombinant wild type Maraba virus at an MOI of 0.05. To assay for cell death, plates were incubated for a further 48 hours and then resazurin was added to a concentration of bug/ml. After 4 hours, absorbance readings at 605 nm and 575 nm were taken to monitor reduction of resazurin to resorufin as a measure of cell viability (O'Brien et al., 2000, Eur J Biochem., 267:5421-6). All screens were performed in duplicate.

Data Analysis: Viability scores for each well were normalized using negative controls (irrelevant siRNA transfection) on a per plate basis. Duplicate screens were averaged on a per well basis. The mean standard deviation for all negative control wells was calculated and used to represent the variability in the assay. Experimental wells which deviated from the mean of all experimental wells by a value equal to 2 standard deviations of the negative controls from their mean were scored as “meaningful”. The mock-infected version of the screen (siRNA alone) was used to remove false positive “hits”. Data was normalized as above. Experimental wells which deviated from the mean of all experimental wells by a value equal to 1 standard deviation of the negative controls from their mean were scored as “meaningful”. Gene targets were designated as “hits” if they were only meaningful in the virus infected (and not in the virus uninfected) screens.

Validation experiments: Several experiments were performed to validate the “hits” identified in the primary siRNA screen. Firstly, we wanted to determine if Maraba virus infection could induce a UPR response. By definition, the “hits” derived from the screen were dependent on virus infection. Therefore, we predicted that the virus must be inducing an ER stress which was not present during the siRNA alone control arm of the screen. There are three arms to the UPR response: (1) ATF6 (2) IRE1 and (3) PERK. Each is known to sense unfolded protein load within the ER by a BIP dependent mechanism. We infected U373 and OVCAR8 cells with Maraba virus and assayed the kinetics of the 3 arms of the UPR response (FIG. 4 panel A&B). As expected, virus infection led to robust ATF6 cleavage indicative of ATF activation during ER stress and persisted throughout the infection (FIG. 4A). IRE1 activation was observed at 4 hours post infection and had subsided 24 hrs post stress. eIF2 alpha is phosphorylated by several protein kinases in response to a variety of stresses (FIG. 4 B). Tunicamycin is seen inducing elF2alpha phosphorylation presumably by the protein kinase PERK (FIG. 4A). We see a robust induction of elF2alpha phosphorylation following Maraba virus infection and we presume that at least some of this activity is through the PERK kinase. Thus Maraba virus infection does indeed elicit an ER stress response. Finally we sought to validate IRE1 as an ER stress response gene whose down-modulation would result in sensitization to the ER stress induced by Maraba virus infection. We targeted IRE1alpha (also known as ERN1) and IRE1beta (also known as ERN2) using siRNA from another vendor (Qiagen USA) across a panel of human cancer cell lines. Subsequent infection with increasing amounts of virus determined the dose response curve for each cell line. FIG. 3C demonstrates the efficiency of siRNA knockdown using this methodology. From this experiment, it was determined that the optimal siRNA concentration for the validation experiments would be 10 nM. FIG. 3A shows a typical curve demonstrating sensitization of a human glioblastoma cell line (SF295) to Maraba infection by knocking down either IRE1 alpha or IRE1beta relative to an irrelevant siRNA control. The summary of the experiment is presented in FIG. 3B, showing that the vast majority of tumour lines tested are sensitized to Maraba virus infection when IRE1 is downregulated. Importantly, GM38 primary human fibroblasts were not sensitized to Maraba virus infection. This demonstrates a tumour specific sensitization when modulating ER stress responses. We propose that this tumour specific sensitization will help target Maraba oncolytic virus destruction to the tumour and spare the surrounding normal parenchyma.

Cell culture: For immunoblot and RT-PCR experiments, U373 (2×10⁵) and OVCAR8 (5×10⁵) cells were seeded in 35 mm plates and grown overnight in complete DMEM. The following morning, tunicamycin (5 μg/mL) and Maraba virus (MOI 5) were diluted in fresh DMEM and added to the cells. Cell pellets were collected at the indicated timepoints post-treatment, washed twice in cold PBS with complete protease inhibitors (Roche) and stored at −80° C. until lysis. For viability experiments, 5×10³ cells were seeded in 96-well plates and grown overnight in complete DMEM. The following morning, siRNA knock-down was performed using RNAimax reagent (Invitrogen) and chemical duplexes specific to human IRE1α (ERN1) or β (ERN2) or a non-targeting (NT) control (Qiagen). After 72 hours, log-dilutions of Maraba virus were added (in triplicate), and 48-72 hours later cell viability was analyzed using the alamar blue method.

Immunoblotting: Total cell lysis buffer (50 mM Tris-HCl; 150 mM NaCl; 1% Triton X-100; 1% SDS) was added to cell pellets, and the lysates were “sheared” using a p100 pipette tip. Total cell lysates were prepared in SDS sample buffer, and 5-50 ug of total protein was separated by SDS-PAGE on 10% Bis-Tris gels and transferred to PVDF membranes. Membranes were probed with primary antibodies diluted in 5% skim milk powder (SMP) overnight at 4° C., followed by horse radish peroxidase-conjugated secondary antibodies diluted in 5% SMP for 1 h at room temperature. Membranes were then treated with ECL reagent, exposed to X-ray film and developed (Kodak X-OMAT 2000A). Primary antibodies used were: ATF-6α (Santa Cruz), p-EIF2α (Cell Signaling), Bip/Grp78 (Cell Signaling), XBP(s) (Biolegend), GAPDH (Advanced Immunochemicals), Maraba viral proteins (anti-VSV).

RT-PCR: Cell pellets were lysed, and RNA extracted using a Qiagen RNAeasy Mini kit. RNA purity and concentration were determined spectrophometrically, and RT-PCR was performed using standard procedures with oligo-dT primers and the following XBP-specific primers:

| (SEQ ID NO: 1) | |
|---|---|
| For: | 5′- cct tgt agt tga gaa cca gg -3'; |
| (SEQ ID NO: 2) | |
| Rev: | 5′- ggg gct tgg tat ata tgt gg -3′. |

The PCR product was separated on a 2% Nusieve/1% Agarose gel and visualized under UV.

### Experiment 2:

### Blockade of ER Stress Response Sensitizes Cancer Cells Towards Viral Oncolysis:

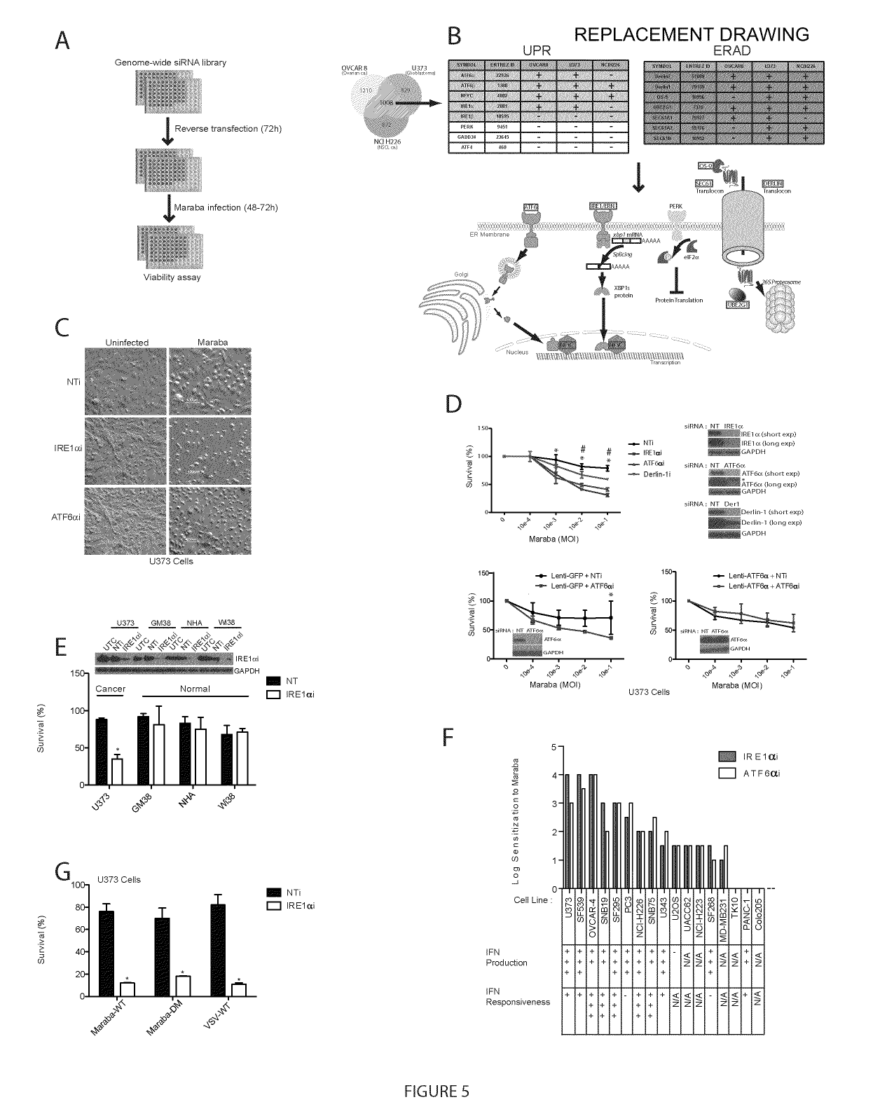

To search for host factors that modulate rhabdovirus-mediated oncolysis, a synthetic lethal RNAi screen of the human genome was performed across three tumour-derived cell lines (FIG. 5a). We used an arrayed library of siRNA pools to target ˜18 500 genes in OVCAR-8 (ovarian carcinoma), U373 (glioblastoma) or NCI-H226 (non-small cell lung carcinoma) cells. Transfected cells were either mock infected or infected with wild type Maraba virus as a representative oncolytic rhabdovirus. Following infection, we incubated the cells for 48-72 h after which we scored cell viability using resazurin vital dye. To identify primary “hits”, we analyzed data from two independent screens for each cell line using the median absolute deviation method⁹. Subtracting those genes scoring positively in the siRNA alone screens defined 1008 synthetic lethal hits common to at least two out of three cancer lines from the primary screen (Table S1). Subsequent bioinformatics analysis revealed a striking enrichment of hits within the ER stress response pathways (FIG. 1B), including members of two of the three known signaling cascades that comprise the unfolded protein response (UPR). Key hits therein included the transcription factors ATF6α and ATF6β, the endoribonucleases/protein kinase IRE1α, and a transcriptional coactivator common to both pathways, NFYC. Together, the ATF6 and IRE1 pathways serve to rescue the ER from an overload of unfolded proteins by increasing chaperone production and ER lipid biogenesis¹⁰. Our screen also identified several members of the SEC61 and the HRD ligase protein translocation complexes (e.g. Derlin-1; FIG. 1B). These proteins are critical for ER-associated degradation (ERAD), which helps rescue an unfolded protein burden by removing misfolded polypeptides from the ER and shuttling them to the 26S proteasome¹¹.

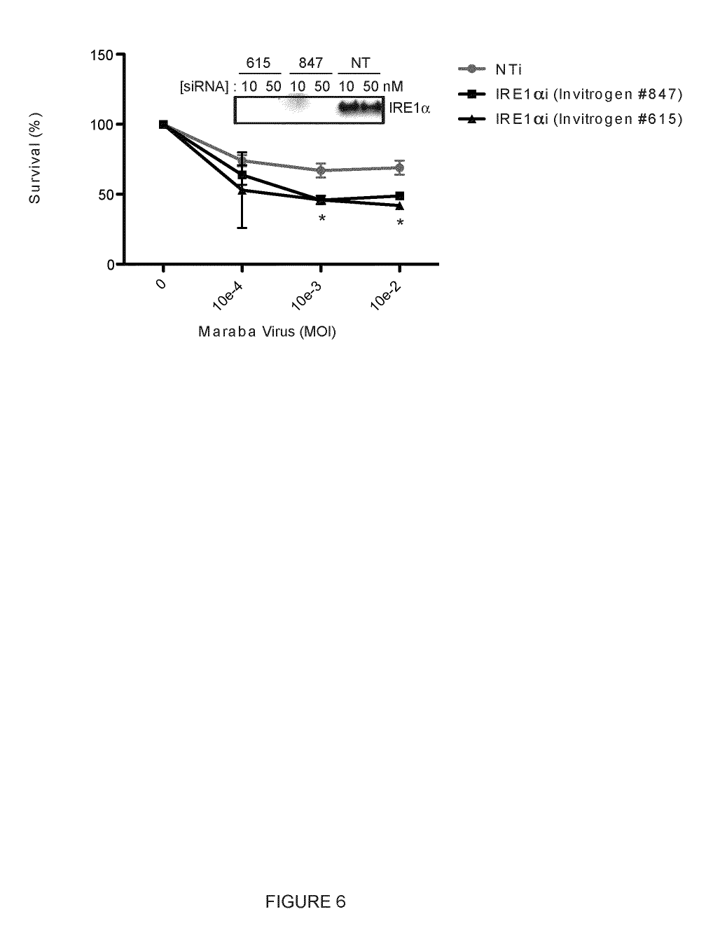

UPR and ERAD components were particularly interesting as sensitizers because ER stress has been reported to be a defining feature of the tumour cell state and components of these pathways are already being pursued as cancer specific targets for stand-alone cancer treatment^{12,13}. We thus performed secondary validation for several members of these pathways, using siRNA with targeting sequences distinct from those employed in the primary screens. Depletion of IRE1α, ATF6α or Derlin-1 significantly sensitized U373 glioblastoma-derived cells to virus-mediated killing across a range of multiplicity of infections (MOI; FIG. 5C-D). To rule out off-target effects, we used multiple siRNA duplexes targeting distinct sequences and consistently found that UPR knockdown sensitized towards viral oncolysis (FIG. 6). In addition, we performed a rescue experiment by ectopically expressing murine ATF6α in U373 cells before depleting its human counterpart. Cells stably expressing mATF6α were completely refractory to the synthetic lethal phenotype associated with oncolytic virus infection and hATF6α knockdown (FIG. 5D). Taken together, these experiments validate our primary screening result that rhabdovirus-mediated oncolysis is greatly enhanced by knocking down components of the UPR and ERAD pathways.

To evaluate therapeutic index, we silenced IRE1α in a small panel of primary human cell lines (GM38 skin fibroblasts, normal human astrocytes (NHA) and Wi38 lung fibroblasts) prior to rhabdovirus infection. In contrast to the pronounced sensitization observed in U373 glioblastoma cells, UPR inhibition did not alter Maraba virus-mediated killing of the normal cell lines (FIG. 5E). We next examined the scope of the synthetic lethal phenotype in a representative subset of the NCI 60 tumour cell panel. RNAi-mediated knockdown of IRE1α or ATF6α significantly sensitized >80% of cancer cell lines tested to virus-mediated killing (FIG. 5F). The sensitized cell lines represent a broad assortment of cancers, some of which had fully effective interferon systems while others had varying degrees of interferon defects (FIG. 5F). Oncolytic rhabdoviruses traditionally have difficulty killing tumour cells with intact interferon responses^{7,8}. Blockade of ER stress responses appears to extend the capability of these viruses to kill such cells, which may result in greater efficacy in the clinical setting where tumours are expected to be more heterogeneous with regards to interferon signaling. Oncolysis by the prototypic oncolytic rhabdovirus VSV and an engineered clinical candidate strain of Maraba virus (Maraba-DM)⁶ was similarly enhanced by UPR inhibition (FIG. 5G). Collectively, these data suggest that the enhancement of virus-mediated oncolysis conferred by inhibiting the ER stress response is tumour cell specific and may have widespread utility across a diverse range of tumour types.

### Synthetic Lethal Interaction Between ER Stress Response Blockade and Rhabdovirus Infection Requires a Preconditioning Process

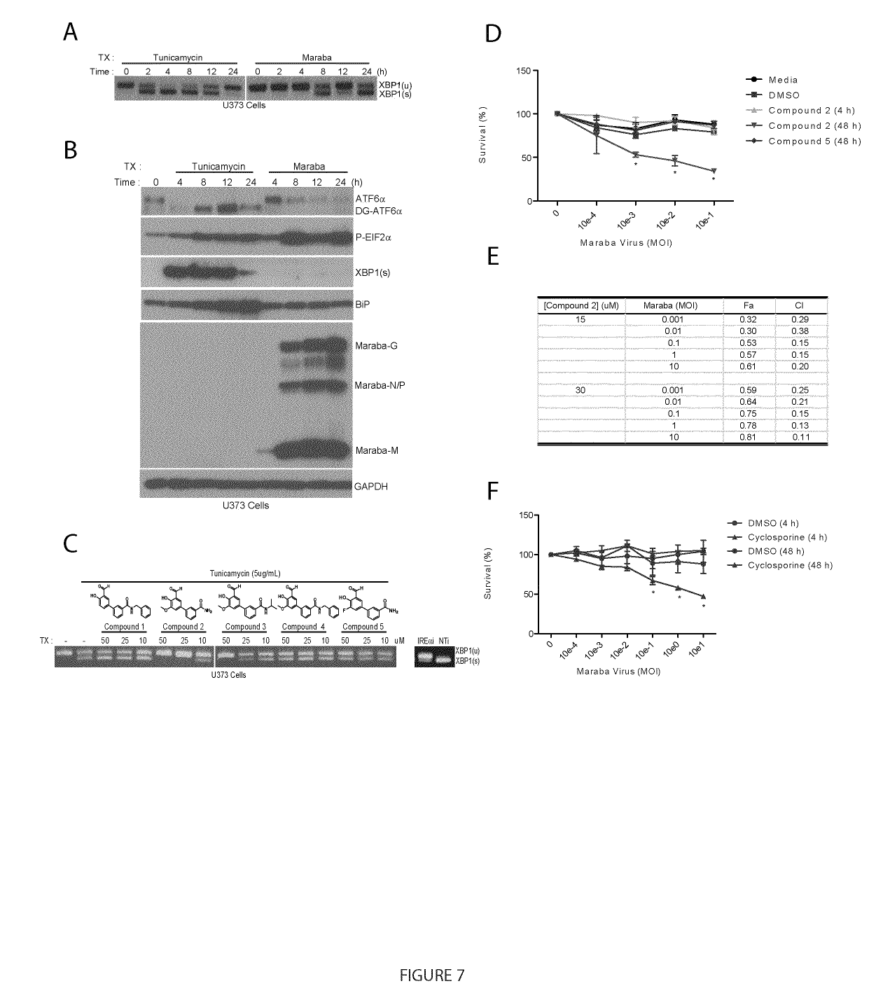

Maraba virus infection caused noticeable ER stress characterized by the activation of the upstream UPR sensors IRE1α (measured by XBP1 mRNA splicing (FIG. 7A)), ATF6α (measured by its cleavage (FIG. 7B)) and PERK (measured by EIF2α phosphorylation (FIG. 7B)). Surprisingly, however, in spite of the activation of these stress sensors, representative downstream UPR effector proteins XBP1(s) and BiP were not elevated after virus infection (FIG. 7B). This result indicates that the UPR was stalled at an early stage during virus infection and thus rendered functionally inert. These data suggest that an inadequate ER stress response is unlikely to be responsible for the observed synthetic lethal interaction between virus and UPR/ERAD knockdown, as the UPR is inhibited rapidly upon virus infection independent of external manipulation. Instead, it appears that sustained inhibition over the 72 h knockdown period is required, and that this may precondition cancer cells to die in response to virus infection.

To test this idea directly, we synthesized a number of compounds that had been reported to inhibit IRE1α¹⁶, along with some novel variants of the original structure. We first tested these compounds for their ability to inhibit XBP1 splicing by IRE1α and found that several were effective in the micromolar range (representative subset depicted in FIG. 7C). We then evaluated the most potent of these, designated Compound 2, and found that it greatly enhanced viral oncolysis in U373 cells when dosed for 48 hours, but not 4 hours (FIG. 7D). Importantly, combination index analyses demonstrated that compound 2 interacted synergistically (CI<1.0) with Maraba virus across a range of doses and MOIs (FIG. 7E). We next treated U373 cells with cyclosporine, a potent inhibitor of the ER chaperone protein cyclophilin B, prior to virus infection. Similar to compound 2, cyclosporine pre-treatment for 48 h but not 4 h greatly enhanced viral oncolysis (FIG. 7F). Together, these drug data demonstrate that the synthetic lethal phenotype is due to a preconditioning process that occurs throughout a period of sustained inhibition of the ER stress response, as opposed to acute blockade.

### ER Preload Rewires Cancer Cells for Apoptosis

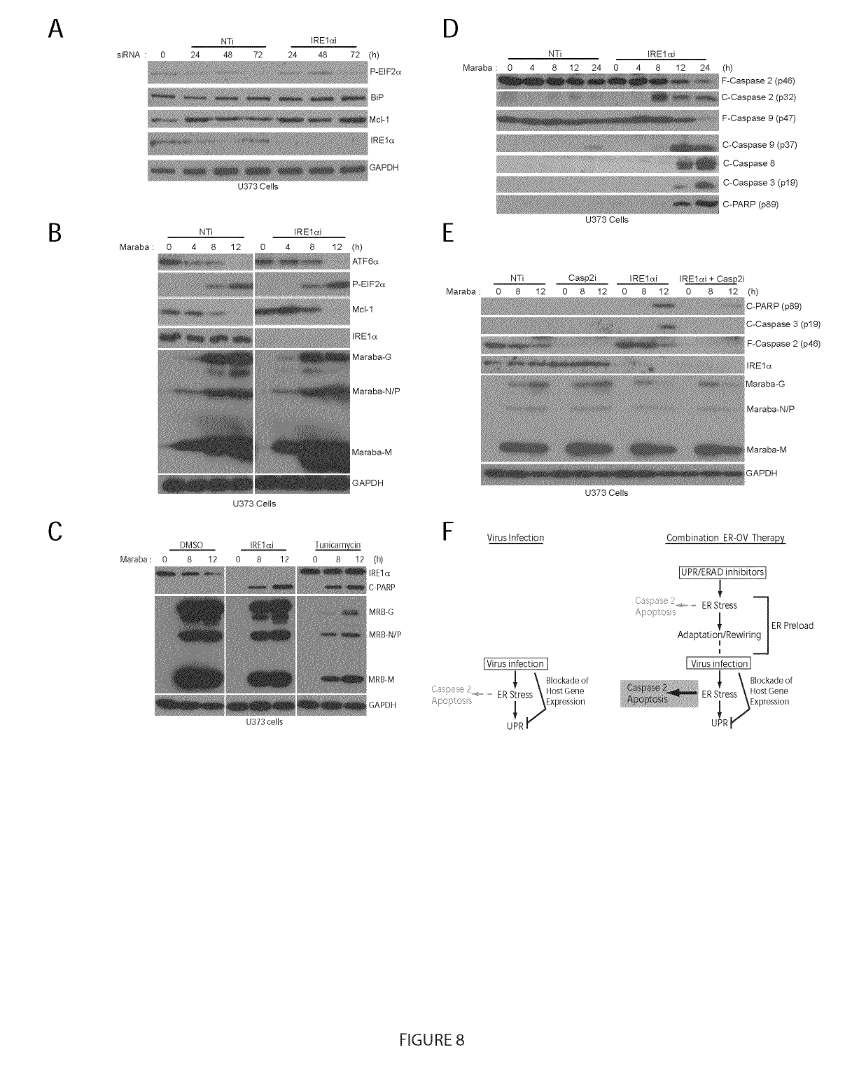

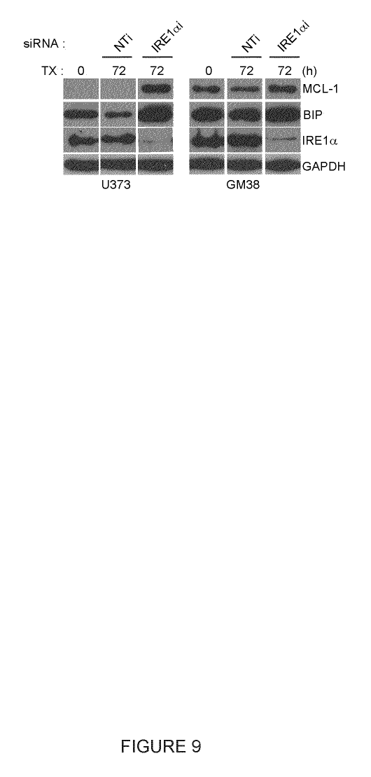

We examined whether inhibiting the ER stress response induces an unfolded protein load prior to viral infection (i.e. “ER preload”). Because there are presently no direct measures of ER protein load per se, we measured activation of the UPR as an indirect readout. Here, IRE1α silencing led to a tumour cell-specific increase in the ER stress-responsive proteins BiP and Mcl-1, as well as a transient induction of the ER stress sensor PERK (as measured by P-EIF2α; FIG. 8A and FIG. 9). These data indicate that IRE1α inhibition resulted in an ER stress in tumour cells. However, this stress response appeared to resolve as eIF2α phosphorylation and ATF6 levels returned to untreated levels, before a period at which virus infection was to be initiated (FIG. 8A 72 h and FIG. 8B 0 h). Additionally we examined whether ER preload altered the kinetics of the ER stress response post-virus infection and observed that loss of IRE1α had no bearing on ATF6α cleavage, EIF2α phosphorylation and Mcl-1 turnover (FIG. 8B), also consistent with our findings that the ER was not under duress at the time of infection. Collectively, these data indicate that inhibiting the ER stress response in tumour cells leads to a transient stress response we refer to here as “ER preload”. We thus asked whether ER preload is accountable for preconditioning cells to respond differently than naïve cells to subsequent oncolytic virus infection. Thus we chemically induced an ER preload by pulsing cells with the glycosylation inhibitor tunicamycin 48 hours before virus infection (FIG. 8C), and found this to also enhance virus-mediated killing. These data are consistent with an ER preload, induced either upon sustained UPR inhibition or ER poisoning, to be requisite for the synthetic lethal phenotype with virus infection.

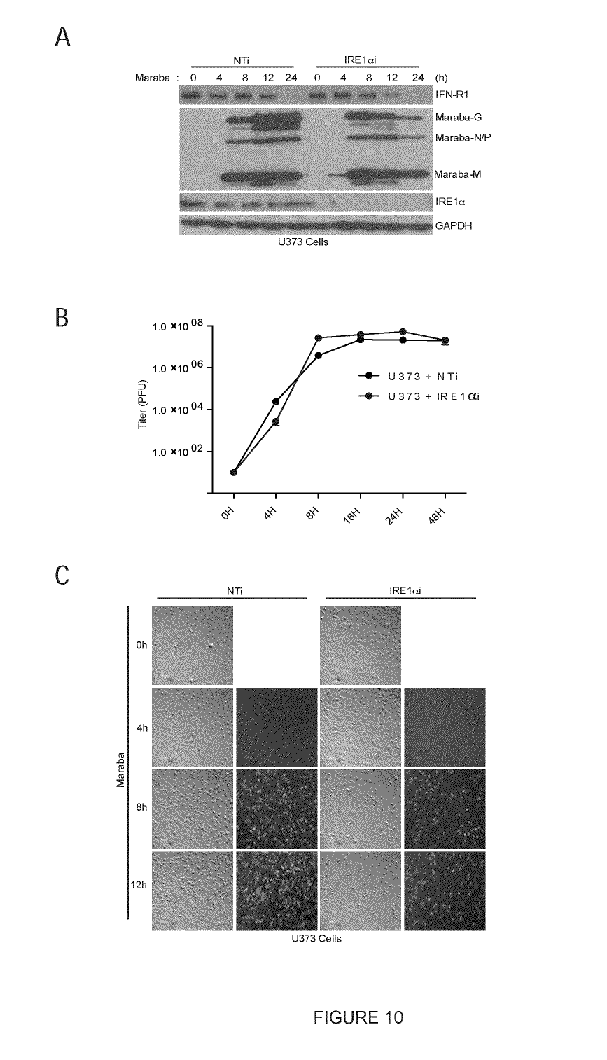

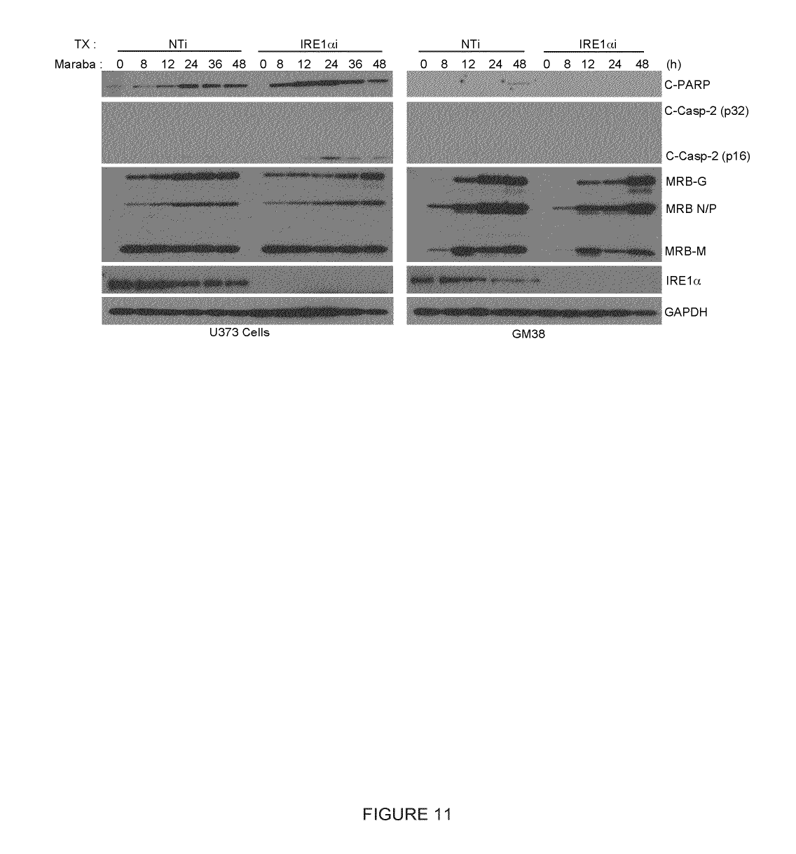

We wished to identify the mechanism of how ER preload might synergize with a subsequent virus infection to promote tumour cell death. We noted that UPR inhibition had no bearing on viral protein expression (FIG. 8B), and confirmed that it also did not alter any aspect of the virus life cycle (FIG. 10). We thus hypothesized that ER preload might “rewire” cancer cell signaling to initiate apoptosis when subsequently challenged with an oncolytic virus. Indeed, IRE1α knockdown greatly enhanced the kinetics of apoptosis during a viral infection, as measured by the cleavage of PARP as well as members of the caspase cascade (FIG. 8D). Notably, caspase 2 was strongly activated in tumour cells by virus infection only when IRE1α was knocked down (FIG. 8D and FIG. 11). Caspase 2 is an initiator caspase that has been implicated in several stress-mediated apoptotic cascades, such as those emanating from DNA damage¹⁸ as well as ER stress¹⁹. It has been reported that this caspase 2 initiated death pathway remains dormant until unresolved ER stress triggers its activation¹⁹. To examine the relevance of its activation, we knocked it down simultaneously with IRE1α and measured apoptosis following virus infection. Remarkably, caspase 2 knockdown largely rescued the synthetic lethal interaction between IRE1α knockdown and virus infection (FIG. 8E). Together, these data suggest that transient ER preload rewires cancer cells to undergo caspase-2 dependent apoptosis upon virus infection (FIG. 8F).

### Chemical Inhibition of IREα Enhances Viral Oncolysis In Vivo

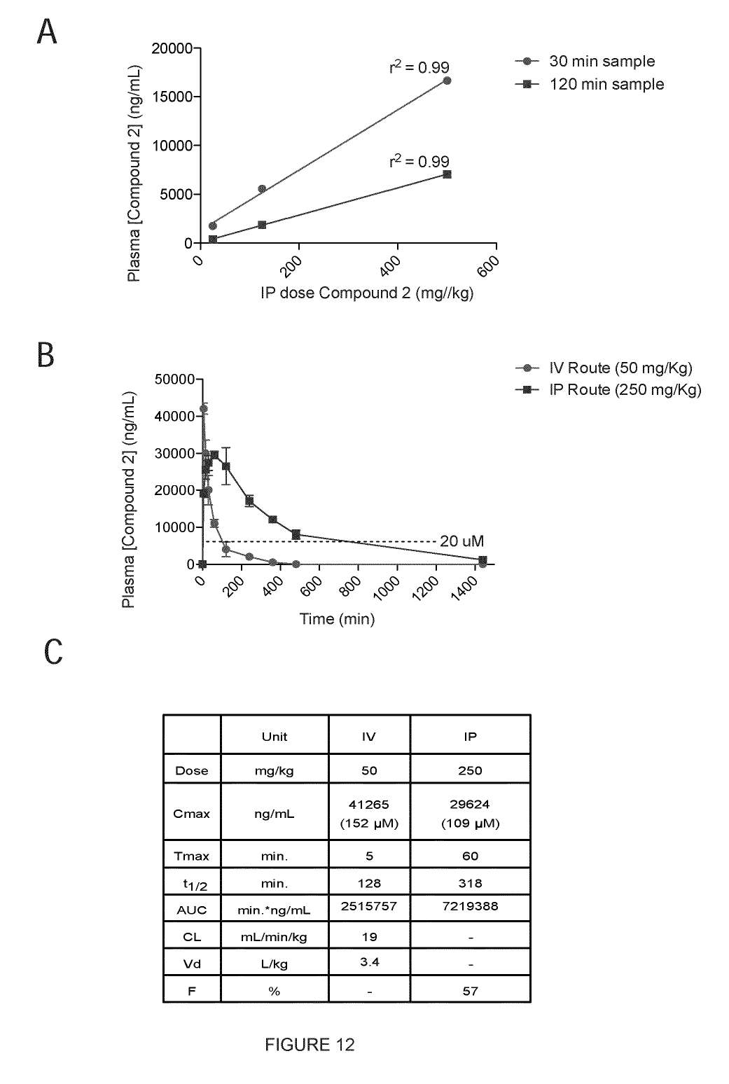

We sought to evaluate the efficacy of pharmacologic ER stress response blockade combined with oncolytic virus therapy in animal models of cancer. To begin, we undertook maximum tolerable dose (MTD) and pharmacokinetic (PK) studies of Compound 2 in CD-1 nude mice. These experiments showed that a single dose of up to 1000 mg/Kg of Compound 2 was tolerated, had a half-life of >6 hours, and had properties consistent with efficient bio-distribution to the extravascular tissues (FIG. 12).

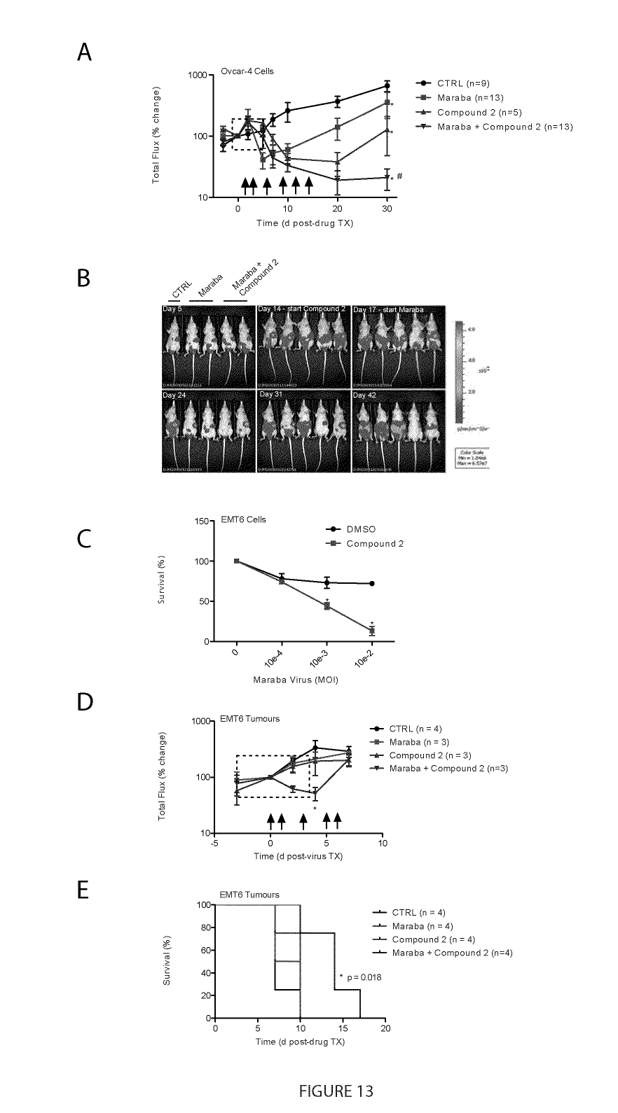

As with many cancers, ovarian carcinoma is difficult to treat clinically due to development of resistance to current therapies²⁰. Thus, to validate our combination therapy approach, we chose a chemoresistant, orthotopic OVCAR-4 xenograft model²¹ that is also refractory to oncolytic virus therapy. OVCAR-4 cells stably expressing firefly luciferase were injected intraperitoneally (IP) into CD-1 nude mice. We monitored tumour growth using in vivo optical imaging, and initiated treatment during the growth phase. To induce ER preconditioning, we treated animals with Compound 2 for three days prior to the first virus injection. Consistent with our findings in cell culture, combination therapy dramatically reduced tumour burden in animal models, an effect that was sustained for >30 days with negligible tumour re-growth (FIG. 13A-B). In contrast, rapid re-growth occurred after an early period of tumour regression using either virus or Compound 2 alone.

As a complement to these experiments in human xenografts, we sought to test this treatment regiment in an immune competent rodent tumour model. In vitro testing determined that the EMT6 breast cancer line, which is particularly resistant to stand-alone rhabdovirus therapy, was significantly sensitized to oncolytic virus killing when pre-treated with Compound 2 (FIG. 13C). Using these cells to generate a tumour model, we confirmed that neither drug nor virus had an appreciable effect on tumour growth as single agents; however, combination therapy significantly reduced tumour burden (FIG. 13D). Notably, when drug treatment was stopped, tumour re-growth occurred even in the presence of continued virus dosing, validating the inter-dependence of the treatment combination. Extending Compound 2 treatment to more than 12 days in combination with virus treatment continued to increase efficacy (FIG. 13E). Taken together, these data demonstrate proof of concept that modulating the ER stress responses can be exploited to enhance oncolytic virus therapy in vivo.

### Materials and Methods:

Cell culturing: Human 293T (American Type Tissue Collection (ATCC)), Monkey Vero (ATCC), murine EMT6 (ATCC), human GM38 (National Institute of General Medical Sciences Mutant Cell Repository, Camden, N.J.), human Wi38 primary fibroblast (ATCC) and cell lines from the NCI 60 cell panel (obtained from the Developmental Therapeutics Program, National Cancer Institute (Bethesda, Md.)) were propagated in Dulbecco's modified Eagle's medium (Hyclone, Logan, Utah) supplemented with 10% fetal calf serum (Cansera, Etobicoke, Ontario, Canada) using standard tissue culture procedures. Normal human astrocytes were propagated in astrocyte media (Sciencecell Research Laboratories) and cultured using standard procedures.

Virus production: Vero cells were plated in 15 cm format, grown to confluence (−2.5×10⁷) and infected with Maraba-WT, Maraba-DM or VSV-WT viruses at MOI 0.1. After 18 h, the virus- containing cell culture media was collected and centrifuged at ˜18,600×g for 1.5 h. The virus pellet was carefully washed and re-suspended in PBS (10 mL), and gently over-layed onto a 20% sucrose solution (1 mL). After ultracentrifugation for 1.5 h (26,900 rpm), the pellet was re-suspended in 15% glucose, aliquoted and stored at −80 deg C.

RNAi screening: An arrayed library of siRNA pools (Dharmacon, Thermo Fisher, USA) was used to target ˜18,500 human genes in OVCAR-8 (ovarian carcinoma), U373 (glioblastoma) or NCI-H226 (non-small cell lung carcinoma) cells. Tumour cells were seeded in 384 well plates (OVCAR-8=1250 cells/well, U373=625 cells/well, NCI-H226=625 cells/well) and allowed to grow for 24 h. Each plate had additional control wells with a non-targeting control siRNA (Dharmacon non-targeting Pool #2) to measure the effect of siRNA transfection on infection, and siRNA targeting PLK-I (Dharmacon) was used to monitor knockdown efficiency. Quadruplicate plate sets were reverse transfected with siRNA (10 nM) using RNAimax (Invitrogen, USA) and incubated for 72 h. From these, duplicate sets of plates were either mock infected or infected with wild type Maraba virus (MOI: OVCAR-8=0.1, U373=0.5, NCI-H226=0.1). Infections were incubated for 48 h (OVCAR-8) or 72 h (U373 and NCI-H226) after which resazurin dye (20 μg/mL) was added to each well, incubated for 6 h and assayed for absorbance (573 nm) to score cell viability.

Data Analysis: Cell viability data from the screens was normalized on a per plate basis using the Median Absolute Deviation (MAD) method (1). Briefly, for each well on the plate, an absolute deviation from the plate median (WAD) was calculated using the formula:

WAD=(well value−plate median excluding controls)

A MAD was calculated for each plate using the formula:

Plate MAD=1.4826* median (WAD)

A MAD score for each gene target (gMAD Score) was calculated as follows:

gMAD Score=average of 2 euplicates (WAD/Plate MAD)

A composite gMAD score for each gene target was derived by subtracting the gMAD scores from the mock-infected screens from infected screens, for each cell line. Gene targets scoring less than −1.85 were considered synthetic lethal hits. Hit lists were derived for each cell line and then compared using VENNY (2) to obtain a final list of hits identified in at least 2 out of the 3 cell lines screened (1008 hits; Table 2). Bioinformatics analysis of the composite hit list was performed using a combination of PANTHER (3), DAVID (4), Ingenuity Pathway Analysis (Ingenuity Systems, USA), and manually curated to identify signaling pathways enriched with hits and to annotate hits for gene function and sub-cellular localization.

RNAi reagents for secondary screening: For all RNAi experiments, the following mRNA sequences were targeted with chemically-synthesized duplexes: IRE1α, 5′-cag cac gga cgt caa gtt tga-3′ (Qiagen) (SEQ ID NO: 3); ATF6α, 5′-cag caa cca att atc agt tta-3′ (Qiagen) (SEQ ID NO: 4); Derlin-1, 5′-tcc cgg cga tca cgc gct att ggt t-3′ (Invitrogen) (SEQ ID NO: 5); Caspase 2 (Dharmacon Smart Pool); Non-targeting (NT) #1, 5′-gca cca tgc ctt tga gct t-3′ (Invitrogen) (SEQ ID NO: 6); NT #2 (Dharmacon NT pool #1). For experiments in FIG. 6, the following sequences were targeted: IRE1α, 5′-ccc tac cta cac ggt gga cat ctt t-3′ (Invitrogen #615) (SEQ ID NO: 7); IRE1α, 5′-gac ctg cgt aaa ttc agg acc tat a-3′ (Invitrogen #847) (SEQ ID NO: 8). All RNAi transfections were performed using RNAimax (Lipofectamine) and left for 72 h before further manipulation. Experiments were done using a [siRNA]=10 nM, except caspase 2 RNAi experiments, which were done at 50 nM.

In vitro cytotoxicity assays with RNAi: Cells were seeded onto 96 well plates to a confluence of ˜50%. The following day, siRNA transfections were performed, and 72 h later the cells were infected at log 10 dilutions with wild type Maraba virus (except for FIG. 5G, which used the indicated viruses). After 48-72 h of infection (depending on the cell line), Resazurin sodium salt (Sigma-Aldrich) was added to a final concentration of 20 μg/ml. After a six-hour incubation, the absorbance was read at a wavelength of 573 nm. To determine “log sensitization”, kill curves were plotted on a log 10/linear graph and EC50 values determined. The log sensitization was calculated by subtracting the EC50 of UPR targeted from non-targeted cell lines, and is represented as log 10 values.

Lentiviral production and rescue experiments: Total RNA was extracted from C2C12 myoblasts using RNeasy technology (Qiagen), and reverse transcribed using random hexamers and Superscript II (Invitrogen). The following primers were used to PCR amplify mouse ATF6α from this cDNA library: Forward, 5′-ggt acc gcg ggc gcg cca tgg agt cgc ctt tta ctc cgg-3′ (SEQ ID NO: 9); Reverse, 5′-ctt gga tcc gcg gcc tac tgc aac gac tca ggg atg-3′ (SEQ ID NO: 10). PCR amplicons were cloned into a pLEX lentiviral vector (Open Bioststems) using the In-Fusion Advantage PCR cloning kit (Clontech). Lentivirus particles were produced by reverse transfecting pDY-ATF6α, pCMV 8.74, and pMD2-G vectors (Fugene-6 transfection reagent, Roche) into 293-T cells. After 72 h, the virus-containing media was removed, passed through a 0.45 μM filter, aliquoted, and frozen at −80 deg C. For rescue experiments, U373 cells were seeded in 6-well format to ˜30% confluence. The following day, lentiviral-containing media was diluted 1:1 with complete media and polybrene was added to a final concentration of 6 μg/mL. Diluted media was added to cells, and plates were spun at 400×g for 1 hour. The following day, siRNA transfections were performed and 72 h later Maraba-WT virus was used to infect the cells. A Resazurin sodium salt cytotoxicity assay was performed 48 h post-infection.

Immunoblotting: Cells were lysed (50 mM Tris-HCl; 150 mM NaCl; 1% Triton X-100; 1% SDS) and protein quantified using the Lowry assay (Bio-Rad). Total cell lysates were prepared in SDS sample buffer, and 5-50 μg of total protein was separated by SDS-PAGE on Bis- Tris gels (ranging from 8-15%) and transferred to nitrocellulose or PVDF membranes. Membranes were probed with primary antibodies diluted in 5% skim milk powder (SMP) or 5% Bovine Serum Albumen (BSA) overnight at 4 deg C., followed by horse radish peroxidase-conjugated secondary antibodies diluted in 5% SMP for 1 h at room temperature. The following primary antibodies were used: rabbit mAb anti-IRE1α (Cell Signaling 14C10); rabbit anti-ATF-6α (Santa Cruz Biotechnology, H-280); rabbit anti-Derlin-1 (Sigma); mouse anti-GAPDH (R&D Systems); rabbit anti-XBP1 (BioLegend, Poly6195); rabbit anti-BIP (Cell signaling); rabbit anti-phospho-EIF2α (Cell signaling); rabbit anti-VSV; rabbit anti-Mcl-1 (Santa Cruz Biotechnology, S-19); goat anti-human IFN cup α/β R1 (R&D S_{y}stems); rat anti-Caspase-2 (Chemicon, 11B4); rabbit anti-Caspase-3 (Cell Signaling, Asp175); rabbit anti-Caspase-9 (Cell Signaling, human-specific); rabbit anti-PARP (Cell Signaling). Finally, proteins were visualized using SuperSignal West Pico Chemiluminescent Substrate System (Pierce Biotechnology).

Interferon production assay: An indirect “interferon production bioassay” was used to estimate the degree to which our cell lines could produce interferon. The indicated lines were infected with Maraba-Δ51 (MOI 3) to trigger an innate immune response and induce interferon (IFN) production. Eighteen hours later, the interferon-containing media was collected and acid neutralized with 0.25N HCl overnight at 4° C. (to destroy virus particles without affecting interferon cytokines), after which time 0.25 NaOH was added to adjust the pH to 7. In parallel, Vero cells were plated to ˜90% confluence in 96 well format, and the following day incubated with the neutralized media for 24 h prior to infection with Maraba-WT. Interferon secreted from the interrogated cell lines post-Maraba-Δ51 infections would protect the Vero cells from Maraba virus infection, to a degree dependent upon the quantity of interferon produced. After 48 h, survival was quantified using a crystal violet assay (Sigma Aldrich). Briefly, cells were incubated with 1% crystal violet solution, washed, dried, re-suspended in 1% SDS and read at a wavelength of 595 nm.

Interferon responsiveness assay: An indirect “interferon responsive bioassay” was used to estimate the degree to which our cell lines could respond to interferon. PC-3 cells were infected with Maraba-Δ51 (MOI 3) for 18 h to produce interferon, after which time the media was collected and acid neutralized, as described above. The following day, the interferon-containing media was added to the indicated cell lines. Twenty-four hours later, Maraba-WT virus was added at a range of MOIs, and cell viability assays were performed after 48 h of infection. Interferon responsiveness was proportional to the amount of protection conferred by media treatment prior to virus infection.

RT-PCR for XBP1 slicing: Total RNA was extracted from cells using a standard RNeasy spin column kit, as described by the manufacturer (Qiagen). RNA was reverse transcribed to cDNA using Superscript II RT (Invitrogen) following the manufacturer's guidelines. Standard PCR was performed using the following primers: XBP1-F: 5′-cct tgt agt tga gaa cca gg-3′ (SEQ ID NO: 11); XBP1-R; 5′-ggg get tgg tat ata tgt gg-3′ (SEQ ID NO: 12). The PCR products were run out on a 3% agarose gel and visualized with UV imager.

Phase-contrast and fluorescent microscopy: All microscopy was done using a standard dissecting microscope (Nikon SMZ1500). Images were captured using a digital camera (Nikon DXM1200F), and analyzed using computerized software (Nikon ACT software).

Single-step growth curves: U373 cells were seeded into 6-well format at ˜50% confluence, and siRNA transfections were performed the following day. After 72 h, the cells were infected with wild-type Maraba at a multiplicity of infection of 5 pfu/cell for 1 hour. Cells were then washed with PBS and incubated at 37° C. Aliquots (100 μl) were taken at 0, 4, 8, 12, 24, and 48 h time points and titred on Vero cells using a standard plaque assay.

Plaque assays: Vero cells were plated at a density of 5e5 cells per/well of a 6 well dish. The next day, 100 μof serial viral dilutions were prepared and added for 1 hour to Vero cells. After viral adsorption, 2 ml of agarose overlay was added (1:1 1% agarose: 2×DMEM and 20% FCS). Plaques were counted the following day.

Small molecule synthesis: Compounds were synthesized through slight modifications of the methods described in W02008154484. A representative example is given for the synthesis of Compound 2.

Synthesis of Compound 2: A solution of 5.0 g (21.6 mmol) of 5-bromo-2-hydroxy-3-methoxybenzaldehyde, 1.81 mL (1.91 g, 23.8 mmol) of methoxymethyl chloride and 7.53 mL (5.59 g) of diisopropylethylamine (43.28 mmol) of diisopropylethylamine was stirred at ambient temperature in 90 mL of dichloromethane for 3 days. The mixture was concentrated and purified by silica gel chromatography eluting with a gradient of hexanes/ethyl acetate to supply 5.37 g of 5-bromo-3-methoxy-2-(methoxymethoxy)benzaldehyde. A portion of this material (1.0 g, 3.64 mmol) was combined with (3-carbamoylphenyl)boronic acid (0.731 g, 3.64 mmol), potassium phosphate (0.655 g, 6.18 mmol), Pd₂(dba)₃ (33.3 mg, 0.0364 mmol), tricyclohexylphosphine (24.5 mg, 0.0872 mmol), 1,4-dioxane (12.0 mL), and water (6.0 mL) in a microwave vessel and heated in a microwave apparatus for 30 min at 85 deg C. After cooling, the crude reaction mixture was filtered through a pad of Celite, absorbed on to silica gel and purified by silica gel chromatography eluting with 100% ethyl acetate. Fractions showing product were combined and concentrated to give 1.10 g of pure 3′-formyl-5′-methoxy-4′-(methoxymethoxy)-[1,1′-biphenyl]-3-carboxamide. The completion of the synthesis of compound 2 was carried out by dissolving this material in 20 mL tetrahydrofuran and adding 20 mL of 1 N aq. HCl. The mixture was stirred at room temperature under positive nitrogen pressure for 16 h. A yellow precipitate was collected by suction filtration to give, after air drying, the crude product. Trituration using methanol provided 0.59 g of 3′-formyl-4′-hydroxy-5′-methoxy-[1,1′-biphenyl]-3-carboxamide (Compound 2). Analytical data (proton NMR and low resolution electrospray mass spectrometry) was consistent with pure desired product.

Small molecule screening: U373 cells were plated in 6-well format to a confluence of ˜75%. The following day, candidate small molecules were dissolved in DMSO and added directly to the cell culture media at a range of concentrations. After 2 h, tunicamycin (5 μg/mL) was added, and total RNA was collected 4 h later. RNA extraction and RT-PCR for XBP1 splicing were performed as described above.

In vitro cytotoxicity assays with small molecules: Cells were seeded onto 96 well plates to a confluence of ˜50%. The following day, siRNA transfections were performed, or small-molecule IRE1α inhibition was initiated. For the small-molecules, DMSO was used as a vehicle with a [drug]=20-50 μM. Drug treatment occurred for either 4 h (“acute” treatment), or was re-applied at 24 h and left for 48 h total (“chronic” treatment). Following knockdown or chemical inhibition, the cells were infected at log dilutions with the indicated rhabdoviruses. After 48-72 h of infection (depending on the cell line), Resazurin sodium salt was added to a final concentration of 20 μg/ml. After a 6 h incubation the absorbance was read at a wavelength of 573 nm.

Maximum tolerable dose (MTD) and pharmacokinetic (PK) studies in mice: For MTD studies, groups of three CD-1 nude mice (6-8 weeks old) were given a single intraperitoneal (IP) injection of Compound 2 (in log 2 increments, diluted in 10% Tween-80) ranging from −50-1000 mg/Kg. The animals were monitored twice daily for signs of distress, including weight loss, morbidity, and respiratory distress. For PK studies, groups of three CD-1 nude mice (6-8 weeks old) were given a single IP injection of Compound 2 (250 mg/Kg), and blood was taken from the saphenous vein at the indicated timepoints. The blood was centrifuged at 3,000 rpm for 10 min, and plasma collected and frozen (−80 deg C.). Plasma samples were analyzed for Compound 2 using LC-MS. To 10 μL plasma, 20 μL acetonitrile was added, vortexed briefly and centrifuged at 14,000 rpm for 10 min. The clear supernatant was transferred in to a vial for LC-MS analysis. Chromatographic separations were carried out on an Acquity UPLC BEH C18 (2.1×50 mm, 1.7 pm) column using ACQUITY UPLC system. The mobile phase was 0.1% formic acid in water (solvent A) and 0.1% formic acid in acetonitrile (solvent B). A gradient starting at 95% solvent A going to 5% in 4.5 min, holding for 0.5 min, going back to 95% in 0.5 min and equilibrating the column for 1 min was employed. A Waters Xevo QTof MS equipped with an atmospheric pressure ionization source was used for MS analysis. MassLynx 4.1 was used for data analysis. WinNonlin was used to obtain the pharmacokinetic parameters.

Ovarian xenograft model: Human ovarian carcinoma-derived OVCAR-4 cells, adapted for bioluminescent imaging, were injected into 6-8 week old athymic CD-1 nude mice (IP injection, 5×10⁶ cells per mouse). Untreated animals develop measurable abdominal tumours (assessed by IVIS imaging) by 4-7 days, become icteric by 3-4 months and must be euthanized shortly thereafter due to systemic disease as characterized by enlarged cancerous liver and spleen, pale kidneys, and cancerous lymph nodes on the abdominal mesentery. For efficacy experiments, Compound 2 (250 mg/kg; or vehicle (10% Tween-80)) was administered twice daily (IP injections), beginning on day 14 and ending on day 19. Maraba-DM (1×10⁵ pfu per cell) or PBS was injected IV (tail vein) on day 16, 17, 19, 23, 25, 27. Animals were monitored daily for weight loss, morbidity, hind leg paralysis and respiratory distress. Tumour images were captured twice weekly with a Xenogen 200 IVIS system (Caliper LS, USA), and total luminescent flux was analyzed on computerized software (Xenogen).

EMT6 syngeneic breast cancer models: Murine EMT6 breast cancer-derived cells (1×10⁵ per mouse) were adapted for bioluminescent imaging and injected into the fat pad of the right lower breast in 6-8 week old Balb/c mice. Mice developed palpable tumours by 5-7 days, which grew rapidly. At 7 days post-tumour implants, mice were treated with Compound 2 (250 mg/kg, IP) or vehicle, twice daily, for six days. Maraba-DM (1×10⁷ pfu per cell) or PBS was injected IV (tail vein) on day 10, 11, 13, 16, and 17. Animals were monitored daily for piloerection, weight loss, morbidity, hind leg paralysis and respiratory distress. Tumour images were captured twice weekly using a Xenogen 200 IVIS system (Caliper LS, USA), and total luminescent flux was analyzed on computerized software (Xenogen). Mice were euthanized when the total flux exceeded 1×10⁷, which corresponded to a tumour burden of ˜500 mm³ and occurred between 14-17 days in untreated animals.

Statistical analyses: For all statistical analyses except survival curves, one- and two-way ANOVAs were performed followed by a Bonferroni multiple comparison's post-hoc test to derive P values (GraphPad Prism). For survival curves, Mantel-Cox Log rank analysis was used to compare plots (GraphPad Prism).

TABLE 2
Composite list of synthetic lethal hits derived from 3 tumour cell lines

| ENTREZ | | GENE | COMPOSITE MAD SCORE | | |
|---|---|---|---|---|---|
| GENEID | GENE NAME | SYMBOL | OVCAR8 | U373 | NCIH226 |
| 55016 | MEMBRANE-ASSOCIATED RING FINGER (C3HC4) 1 | 38776 | | −2.41151 | −4.10084 |
| 10801 | SEPTIN 9 | 38968 | −2.13128 | −3.66817 | −2.86592 |
| 346288 | FLJ44060 PROTEIN | 38973 | −5.54837 | −3.0341 | |
| 51166 | AMINOADIPATE AMINOTRANSFERASE | AADAT | −2.49835 | −2.95805 | |
| 22848 | AP2 ASSOCIATED KINASE 1 | AAK1 | −2.77645 | −2.01405 | |
| 14 | ANGIO-ASSOCIATED, MIGRATORY CELL PROTEIN | AAMP | −3.11061 | −2.60732 | −2.15765 |
| 15 | ARYLALKYLAMINE N-ACETYLTRANSFERASE | AANAT | −1.95955 | −2.41831 | |
| 60496 | DKFZP566E2346 PROTEIN | AASDHPPT | −2.16412 | −2.38839 | |
| 23456 | ATP-BINDING CASSETTE, SUB-FAMILY B (MDR/TAP), MEMBER 10 | ABCB10 | −2.45154 | −2.10888 | −2.11658 |
| 6833 | ATP-BINDING CASSETTE, SUB-FAMILY C (CFTR/MRP), MEMBER 8 | ABCC8 | −2.20353 | −1.96417 | |
| 64137 | ATP-BINDING CASSETTE, SUB-FAMILY G (WHITE), MEMBER 4 | ABCG4 | −2.13289 | −2.17044 | |
| 80325 | ANKYRIN REPEAT AND BTB (POZ) DOMAIN CONTAINING 1 | ABTB1 | −2.29777 | −2.27845 | −1.93118 |
| 65057 | ADRENOCORTICAL DYSPLASIA HOMOLOG (MOUSE) | ACD | −3.49186 | −4.48169 | |
| 130013 | AMINOCARBOXYMUCONATE SEMIALDEHYDE DECARBOXYLASE | ACMSD | −3.479 | −3.14302 | −2.42407 |
| 134526 | ACYL-COA THIOESTERASE 12 | ACOT12 | −1.99657 | | −3.33543 |
| 8309 | ACYL-COENZYME A OXIDASE 2, BRANCHED CHAIN | ACOX2 | −3.07964 | −3.9036 | −3.83209 |
| 55289 | ACYL-COENZYME A OXIDASE-LIKE | ACOXL | −2.18631 | −2.45197 | |
| 10121 | ARP1 ACTIN-RELATED PROTEIN 1 HOMOLOG A, CENTRACTIN ALPHA | ACTR1A | −3.02305 | −3.39827 | −4.92131 |
| | (YEAST) | | | | |
| 8747 | ADAM METALLOPEPTIDASE DOMAIN 21 | ADAM21 | −1.93391 | −4.53126 | −2.64672 |
| 80070 | ADAM METALLOPEPTIDASE WITH THROMBOSPONDIN TYPE 1 MOTIF, 20 | ADAMTS20 | −2.21441 | −1.90698 | |
| 23536 | ADENOSINE DEAMINASE, TRNA-SPECIFIC 1 | ADAT1 | −1.93515 | −2.22345 | |
| 107 | ADENYLATE CYCLASE 1 (BRAIN) | ADCY1 | | −2.87709 | −2.5232 |
| 111 | ADENYLATE CYCLASE 5 | ADCY5 | −2.69979 | −2.68721 | |
| 123 | ADIPOSE DIFFERENTIATION-RELATED PROTEIN | ADFP | −5.03679 | −2.31512 | −2.44941 |
| 133 | ADRENOMEDULLIN | ADM | −2.40943 | −2.44641 | |
| 84890 | CHROMOSOME 10 OPEN READING FRAME 22 | ADO | −3.38911 | −4.48099 | |
| 140 | ADENOSINE A3 RECEPTOR | ADORA3 | −2.5352 | | −2.76387 |
| 173 | AFAMIN | AFM | −2.2435 | −2.28448 | |
| 10598 | AHA1, ACTIVATOR OF HEAT SHOCK 90 KDA PROTEIN ATPASE | AHSA1 | −2.88922 | −3.59496 | −2.99421 |
| | HOMOLOG 1 (YEAST) | | | | |
| 326 | AUTOIMMUNE REGULATOR (AUTOIMMUNE POLYENDOCRINOPATHY | AIRE | −2.20195 | −3.22496 | |
| | CANDIDIASIS ECTODERMAL DYSTROPHY) | | | | |
| 8852 | A KINASE (PRKA) ANCHOR PROTEIN 4 | AKAP4 | | −2.04143 | −2.57289 |
| 6718 | ALDO-KETO REDUCTASE FAMILY 1, MEMBER D1 (DELTA 4-3- | AKR1D1 | −2.07303 | −4.60704 | |
| | KETOSTEROID-5-BETA-REDUCTASE) | | | | |
| 80216 | KIAA1527 PROTEIN | ALPK1 | −1.95916 | −3.47659 | |
| 151254 | AMYOTROPHIC LATERAL SCLEROSIS 2 (JUVENILE) CHROMOSOME | ALS2CR11 | −4.75935 | −2.53681 | −2.40034 |
| | REGION, CANDIDATE 11 | | | | |
| 258 | AMELOBLASTIN, ENAMEL MATRIX PROTEIN | AMBN | | −3.16187 | −2.15822 |
| 348094 | ANKYRIN REPEAT AND DEATH DOMAIN CONTAINING 1A | ANKDD1A | −2.00511 | | −1.87831 |
| 81573 | ANKYRIN REPEAT DOMAIN 13C | ANKRD13C | −2.89415 | −2.23827 | |
| 84250 | ANKYRIN REPEAT DOMAIN 32 | ANKRD32 | | −2.60824 | −3.37461 |
| 375248 | ANKYRIN REPEAT DOMAIN 36 | ANKRD36 | | −2.80648 | −2.14125 |
| 65124 | CHROMOSOME 2 OPEN READING FRAME 26 | ANKRD57 | −2.19538 | | −2.57045 |
| 307 | ANNEXIN A4 | ANXA4 | −2.47948 | −2.7168 | |
| 164 | ADAPTOR-RELATED PROTEIN COMPLEX 1, GAMMA 1 SUBUNIT | AP1G1 | | −2.98113 | −3.03354 |
| 160 | ADAPTOR-RELATED PROTEIN COMPLEX 2, ALPHA 1 SUBUNIT | AP2A1 | −2.0658 | −4.29449 | −2.4135 |
| 161 | ADAPTOR-RELATED PROTEIN COMPLEX 2, ALPHA 2 SUBUNIT | AP2A2 | −2.29654 | −3.52961 | |
| 11154 | ADAPTOR-RELATED PROTEIN COMPLEX 4, SIGMA 1 SUBUNIT | AP4S1 | −2.43482 | −3.22071 | −3.08222 |
| 147495 | ADENOMATOSIS POLYPOSIS COLI DOWN-REGULATED 1 | APCDD1 | −3.70917 | −3.66013 | |
| 8539 | APOPTOSIS INHIBITOR 5 | API5 | −2.95141 | | −2.25814 |
| 351 | AMYLOID BETA (A4) PRECURSOR PROTEIN (PEPTIDASE NEXIN-II, | APP | −2.21138 | −2.30784 | −2.14111 |
| | ALZHEIMER DISEASE) | | | | |
| 361 | AQUAPORIN 4 | AQP4 | | −1.99469 | −4.23735 |
| 27236 | ADP-RIBOSYLATION FACTOR INTERACTING PROTEIN 1 (ARFAPTIN 1) | ARFIP1 | −4.11551 | −2.80184 | −1.9262 |
| 392 | RHO GTPASE ACTIVATING PROTEIN 1 | ARHGAP1 | −2.91216 | −2.5815 | |
| 55843 | RHO GTPASE ACTIVATING PROTEIN 15 | ARHGAP15 | | −1.97386 | −2.25784 |
| 84986 | RHO GTPASE ACTIVATING PROTEIN 19 | ARHGAP19 | −2.63195 | −2.99612 | |
| 9138 | RHO GUANINE NUCLEOTIDE EXCHANGE FACTOR (GEF) 1 | ARHGEF1 | −3.08806 | −5.5479 | −4.87465 |
| 340485 | N-ACYLSPHINGOSINE AMIDOHYDROLASE 3-LIKE | ASAH3L | −2.97451 | −3.77336 | −2.38993 |
| 79827 | ADIPOCYTE-SPECIFIC ADHESION MOLECULE | ASAM | −2.0148 | | −2.78807 |
| 430 | ACHAETE-SCUTE COMPLEX-LIKE 2 (DROSOPHILA) | ASCL2 | −2.22619 | −2.21252 | −1.90579 |
| 79058 | ALVEOLAR SOFT PART SARCOMA CHROMOSOME REGION, CANDIDATE 1 | ASPSCR1 | | −2.00368 | −2.08419 |
| 22926 | ACTIVATING TRANSCRIPTION FACTOR 6 | ATF6 | −2.79393 | −2.93049 | |
| 491 | ATPASE, CA++ TRANSPORTING, PLASMA MEMBRANE 2 | ATP2B2 | −2.37995 | −2.62897 | |
| 27032 | ATPASE, CA++ TRANSPORTING, TYPE 2C, MEMBER 1 | ATP2C1 | −2.66675 | −2.55357 | −3.33098 |
| 27109 | ATP SYNTHASE, H+ TRANSPORTING, MITOCHONDRIAL F0 COMPLEX, | ATP5S | −2.94141 | | −2.00102 |
| | SUBUNIT S (FACTOR B) | | | | |
| 79895 | ATPASE, CLASS I, TYPE 8B, MEMBER 4 | ATP8B4 | | −2.32716 | −1.96282 |
| 11273 | ATAXIN 2-LIKE | ATXN2L | −2.16286 | −2.90014 | −3.3262 |
| 9212 | AURORA KINASE B | AURKB | −2.13449 | −1.86738 | |
| 10677 | ADVILLIN | AVIL | −2.41928 | −2.9485 | |
| 553 | ARGININE VASOPRESSIN RECEPTOR 1B | AVPR1B | −1.90893 | | −2.64188 |
| 64651 | AXIN1 UP-REGULATED 1 | AXUD1 | −2.62194 | −2.01919 | |
| 8708 | UDP-GAL:BETAGLCNAC BETA 1,3-GALACTOSYLTRANSFERASE, | B3GALT1 | −1.87025 | | −2.13837 |
| | POLYPEPTIDE 1 | | | | |
| 8707 | UDP-GAL:BETAGLCNAC BETA 1,3-GALACTOSYLTRANSFERASE, | B3GALT2 | −2.49262 | −2.78836 | |
| | POLYPEPTIDE 2 | | | | |
| 11285 | XYLOSYLPROTEIN BETA 1,4-GALACTOSYLTRANSFERASE, | B4GALT7 | −3.46031 | −3.70379 | |
| | POLYPEPTIDE 7 (GALACTOSYLTRANSFERASE I) | | | | |
| 54971 | BTG3 ASSOCIATED NUCLEAR PROTEIN | BANP | −4.91691 | −3.03484 | |
| 55212 | BARDET-BIEDL SYNDROME 7 | BBS7 | −3.48742 | −1.98035 | |
| 64919 | B-CELL CLL/LYMPHOMA 11B (ZINC FINGER PROTEIN) | BCL11B | | −2.28374 | −2.3214 |
| 602 | B-CELL CLL/LYMPHOMA 3 | BCL3 | −2.09542 | | −1.86381 |
| 255877 | B-CELL CLL/LYMPHOMA 6, MEMBER B (ZINC FINGER PROTEIN) | BCL6B | −2.32078 | −2.67158 | |
| 605 | B-CELL CLL/LYMPHOMA 7A | BCL7A | | −3.31142 | −2.31437 |
| 331 | BACULOVIRAL IAP REPEAT-CONTAINING 4 | BIRC4 | −2.8144 | −2.34593 | |
| 652 | BONE MORPHOGENETIC PROTEIN 4 | BMP4 | −2.51609 | −2.03764 | −2.64151 |
| 23246 | BLOCK OF PROLIFERATION 1 | BOP1 | −2.4163 | −2.84961 | |
| 6046 | BROMODOMAIN CONTAINING 2 | BRD2 | −3.70807 | | −2.19798 |
| 56853 | BRUNO-LIKE 4, RNA BINDING PROTEIN (DROSOPHILA) | BRUNOL4 | −2.38575 | −3.7975 | −3.28405 |
| 138151 | BTB (POZ) DOMAIN CONTAINING 14A | BTBD14A | −5.42149 | −4.98281 | −4.37291 |
| 7832 | BTG FAMILY, MEMBER 2 | BTG2 | −2.26701 | | −2.9354 |
| 221060 | CHROMOSOME 10 OPEN READING FRAME 111 | C10ORF111 | −3.52161 | −2.1038 | |
| 55088 | CHROMOSOME 10 OPEN READING FRAME 118 | C10ORF118 | | −2.14738 | −2.08959 |
| 143384 | CHROMOSOME 10 OPEN READING FRAME 46 | C10ORF46 | −2.05153 | | −2.51401 |
| 79741 | CHROMOSOME 10 OPEN READING FRAME 68 | C10ORF68 | | −2.00775 | −2.09128 |
| 79946 | CHROMOSOME 10 OPEN READING FRAME 95 | C10ORF95 | −4.25451 | −2.45897 | |
| 79081 | CHROMOSOME 11 OPEN READING FRAME 48 | C11ORF48 | −6.50729 | −3.88765 | |
| 84067 | CHROMOSOME 11 OPEN READING FRAME 56 | C11ORF56 | −4.29459 | −2.6957 | −4.52064 |
| 745 | CHROMOSOME 11 OPEN READING FRAME 9 | C11ORF9 | −4.1401 | −2.58869 | |
| 283416 | CHROMOSOME 12 OPEN READING FRAME 61 | C12ORF61 | −2.30163 | −1.9713 | −2.30156 |
| 221150 | CHROMOSOME 13 OPEN READING FRAME 3 | C13ORF3 | −3.40649 | −4.80828 | |
| 55668 | CHROMOSOME 14 OPEN READING FRAME 118 | C14ORF118 | −2.21825 | −2.63428 | |
| 64430 | CHROMOSOME 14 OPEN READING FRAME 135 | C14ORF135 | −2.08351 | −1.97923 | |
| 54675 | CHROMOSOME 20 OPEN READING FRAME 155 | CRLS1 | −5.73605 | −2.74203 | |
| 1414 | CRYSTALLIN, BETA B1 | CRYBB1 | −3.42872 | −2.10112 | |
| 1429 | CRYSTALLIN, ZETA (QUINONE REDUCTASE) | CRYZ | −2.52933 | −3.09796 | −2.20554 |
| 8531 | COLD SHOCK DOMAIN PROTEIN A | CSDA | −2.30656 | −5.08058 | −2.30271 |
| 1437 | COLONY STIMULATING FACTOR 2 (GRANULOCYTE-MACROPHAGE) | CSF2 | | −3.23829 | −2.10723 |
| 1452 | CASEIN KINASE 1, ALPHA 1 | CSNK1A1 | −2.31878 | −2.18873 | |
| 122011 | CASEIN KINASE 1, ALPHA 1-LIKE | CSNK1A1L | | −1.85463 | −1.90398 |
| 1485 | CANCER/TESTIS ANTIGEN 1B | CTAG1B | −2.16637 | | −1.86576 |
| 56474 | CTP SYNTHASE II | CTPS2 | −2.45539 | −2.23267 | −2.90417 |
| 1519 | CATHEPSIN O | CTSO | −2.81322 | | −3.55489 |
| 55917 | CTTNBP2 N-TERMINAL LIKE | CTTNBP2NL | −2.69729 | | −2.50301 |
| 2919 | CHEMOKINE (C—X—C MOTIF) LIGAND 1 (MELANOMA GROWTH | CXCL1 | −2.43419 | −3.48441 | |
| | STIMULATING ACTIVITY, ALPHA) | | | | |
| 80319 | CXXC FINGER 4 | CXXC4 | −3.34186 | −3.9532 | |
| 1588 | CYTOCHROME P450, FAMILY 19, SUBFAMILY A, POLYPEPTIDE 1 | CYP19A1 | −2.68268 | −2.25115 | −2.76115 |
| 51302 | CYTOCHROME P450, FAMILY 39, SUBFAMILY A, POLYPEPTIDE 1 | CYP39A1 | −2.56803 | −1.99217 | |
| 284541 | CYTOCHROME P450, FAMILY 4, SUBFAMILY A, POLYPEPTIDE 22 | CYP4A22 | | −3.73785 | −3.26585 |
| 27351 | DNA SEGMENT, CHR 15, WAYNE STATE UNIVERSITY 75, EXPRESSED | D15WSU75E | −2.14509 | | −1.88014 |
| 2532 | DUFFY BLOOD GROUP, CHEMOKINE RECEPTOR | DARC | −2.71008 | −4.47006 | |
| 440097 | DEVELOPING BRAIN HOMEOBOX 2 | DBX2 | −4.3273 | −3.66309 | |
| 1632 | DODECENOYL-COENZYME A DELTA ISOMERASE (3,2 TRANS-ENOYL- | DCI | −2.35369 | −1.99944 | |
| | COENZYME A ISOMERASE) | | | | |
| 9201 | DOUBLECORTIN AND CAM KINASE-LIKE 1 | DCLK1 | | −2.5543 | −2.68774 |
| 64421 | DNA CROSS-LINK REPAIR 1C (PSO2 HOMOLOG, S. CEREVISIAE) | DCLRE1C | −1.96141 | | −2.11474 |
| 1638 | DOPACHROME TAUTOMERASE (DOPACHROME DELTA-ISOMERASE, | DCT | −2.41936 | | −2.71413 |
| | TYROSINE-RELATED PROTEIN 2) | | | | |
| 55208 | DCN1, DEFECTIVE IN CULLIN NEDDYLATION 1, DOMAIN CONTAINING 2 | DCUN1D2 | −1.93227 | −1.90673 | |
| | (S. CEREVISIAE) | | | | |
| 80821 | DDHD DOMAIN CONTAINING 1 | DDHD1 | | −2.0493 | −2.87835 |
| 84301 | DDI1, DNA-DAMAGE INDUCIBLE 1, HOMOLOG 2 (S. CEREVISIAE) | DDI2 | −4.31245 | −2.95685 | −3.83909 |
| 55510 | DEAD (ASP-GLU-ALA-ASP) BOX POLYPEPTIDE 43 | DDX43 | | −2.24216 | −3.00093 |
| 10522 | DEFORMED EPIDERMAL AUTOREGULATORY FACTOR 1 (DROSOPHILA) | DEAF1 | −2.17474 | −1.91778 | |
| 1668 | DEFENSIN, ALPHA 1 | DEFA3 | −4.79547 | −2.23812 | |
| 414325 | DEFENSIN, BETA 103B | DEFB103B | −3.21911 | −1.92634 | |
| 140850 | DEFENSIN, BETA 127 | DEFB127 | −2.55821 | −2.07617 | |
| 8560 | DEGENERATIVE SPERMATOCYTE HOMOLOG 1, LIPID DESATURASE | DEGS1 | −2.11252 | −3.53413 | |
| | (DROSOPHILA) | | | | |
| 79139 | DER1-LIKE DOMAIN FAMILY, MEMBER 1 | DERL1 | −2.3016 | −1.87201 | |
| 51009 | DER1-LIKE DOMAIN FAMILY, MEMBER 2 | DERL2 | −3.0246 | −1.95458 | |
| 1676 | DNA FRAGMENTATION FACTOR, 45 KDA, ALPHA POLYPEPTIDE | DFFA | −2.81706 | −4.22012 | −3.21915 |
| 1677 | DNA FRAGMENTATION FACTOR, 40 KDA, BETA POLYPEPTIDE | DFFB | −2.12535 | −3.3819 | |
| | (CASPASE-ACTIVATED DNASE) | | | | |
| 85359 | DIGEORGE SYNDROME CRITICAL REGION GENE 6-LIKE | DGCR6L | −3.38017 | −2.23632 | |
| 8526 | DIACYLGLYCEROL KINASE, EPSILON 64 KDA | DGKE | −3.20331 | −2.93058 | |
| 27294 | DIHYDRODIOL DEHYDROGENASE (DIMERIC) | DHDH | −2.3481 | −2.28125 | |
| 23181 | DIP2 DISCO-INTERACTING PROTEIN 2 HOMOLOG A (DROSOPHILA) | DIP2A | −2.18537 | −2.41581 | −2.37741 |
| 27123 | DICKKOPF HOMOLOG 2 (XENOPUS LAEVIS) | DKK2 | −3.65488 | | −2.91351 |
| 9231 | DISCS, LARGE HOMOLOG 5 (DROSOPHILA) | DLG5 | −2.197 | −2.7603 | |
| 1750 | DISTAL-LESS HOMEOBOX 6 | DLX6 | | −1.86594 | −2.43545 |
| 55567 | DYNEIN, AXONEMAL, HEAVY POLYPEPTIDE 3 | DNAH3 | −2.27884 | | −2.05462 |
| 1769 | DYNEIN, AXONEMAL, HEAVY POLYPEPTIDE 8 | DNAH8 | −2.05019 | | −3.08662 |
| 85479 | DNAJ (HSP40) HOMOLOG, SUBFAMILY C, MEMBER 5 BETA | DNAJC5B | −3.74895 | −2.7117 | |
| 144132 | DYNEIN HEAVY CHAIN DOMAIN 1 | DNHD1 | −1.86308 | | −1.85855 |
| 57572 | DEDICATOR OF CYTOKINESIS 6 | DOCK6 | −2.50006 | | −2.61583 |
| 1797 | DOM-3 HOMOLOG Z (C. ELEGANS) | DOM3Z | −2.77576 | −1.97913 | |
| 84444 | DOTI-LIKE, HISTONE H3 METHYLTRANSFERASE (S. CEREVISIAE) | DOT1L | | −3.22467 | −2.39116 |
| 1801 | DPH1 HOMOLOG (S. CEREVISIAE) | DPH1 | −2.78644 | −3.04536 | −2.96731 |
| 54344 | DOLICHYL-PHOSPHATE MANNOSYLTRANSFERASE POLYPEPTIDE 3 | DPM3 | | −2.62437 | −2.25698 |
| 340168 | DEVELOPMENTAL PLURIPOTENCY ASSOCIATED 5 | DPPA5 | −4.16294 | −2.28648 | −2.10864 |
| 1807 | DIHYDROPYRIMIDINASE | DPYS | −1.99033 | −4.15073 | |
| 1812 | DOPAMINE RECEPTOR D1 | DRD1 | −2.6749 | −1.91894 | −2.65582 |
| 1814 | DOPAMINE RECEPTOR D3 | DRD3 | −4.81514 | −3.38421 | |
| 1826 | DOWN SYNDROME CELL ADHESION MOLECULE | DSCAM | −1.8794 | −2.99691 | |
| 1828 | DESMOGLEIN 1 | DSG1 | −1.86862 | −3.18021 | |
| 1832 | DESMOPLAKIN | DSP | −4.3071 | | −2.18346 |
| 80824 | DUAL SPECIFICITY PHOSPHATASE 16 | DUSP16 | −2.10263 | −2.55858 | |
| 63904 | DUAL SPECIFICITY PHOSPHATASE 21 | DUSP21 | | −3.15806 | −2.5002 |
| 84332 | HYPOTHETICAL PROTEIN MGC16186 | DYDC2 | | −2.38669 | −2.35023 |
| 1783 | DYNEIN, CYTOPLASMIC 1, LIGHT INTERMEDIATE CHAIN 2 | DYNC1LI2 | −4.31596 | −2.06078 | |
| 1859 | DUAL-SPECIFICITY TYROSINE-(Y)-PHOSPHORYLATION REGULATED | DYRK1A | −2.69878 | −3.14822 | −1.97728 |
| | KINASE 1A | | | | |
| 1877 | E4F TRANSCRIPTION FACTOR 1 | E4F1 | −2.17668 | | −2.6409 |
| 1889 | ENDOTHELIN CONVERTING ENZYME 1 | ECE1 | −2.5034 | −2.16589 | |
| 79746 | ENOYL COENZYME A HYDRATASE DOMAIN CONTAINING 3 | ECHDC3 | −3.73688 | −4.15934 | |
| 112399 | HYPOTHETICAL PROTEIN FLJ21620 | EGLN3 | −2.76447 | −4.40225 | |
| 126272 | EID-2-LIKE INHIBITOR OF DIFFERENTIATION-3 | EID2B | −3.34573 | −2.33512 | −2.44539 |
| 440275 | EUKARYOTIC TRANSLATION INITIATION FACTOR 2 ALPHA KINASE 4 | EIF2AK4 | | −4.47732 | −3.08146 |
| 7458 | WILLIAMS-BEUREN SYNDROME CHROMOSOME REGION 1 | EIF4H | −3.13161 | −4.5717 | −2.33113 |
| 23436 | ELASTASE 3A, PANCREATIC | ELA3B | −2.87901 | −2.22243 | |
| 114794 | KIAA1904 PROTEIN | ELFN2 | −3.93848 | −3.94671 | |
| 10436 | EMG1 NUCLEOLAR PROTEIN HOMOLOG (S. CEREVISIAE) | EMG1 | −4.78692 | −4.19447 | |
| 2009 | ECHINODERM MICROTUBULE ASSOCIATED PROTEIN LIKE 1 | EML1 | −4.52565 | | −2.91035 |
| 27436 | ECHINODERM MICROTUBULE ASSOCIATED PROTEIN LIKE 4 | EML4 | | −3.38095 | −1.99126 |
| 9941 | ENDONUCLEASE G-LIKE 1 | ENDOGL1 | −2.34432 | −1.91204 | −2.4438 |
| 55068 | PROLIFERATION-INDUCING PROTEIN 38 | ENOX1 | −3.15056 | −5.20001 | −2.74843 |
| 339221 | ECTONUCLEOTIDE PYROPHOSPHATASE/PHOSPHODIESTERASE 7 | ENPP7 | | −3.43628 | −1.85947 |
| 957 | ECTONUCLEOSIDE TRIPHOSPHATE DIPHOSPHOHYDROLASE 5 | ENTPD5 | −5.07624 | −3.65826 | |
| 56943 | ENHANCER OF YELLOW 2 HOMOLOG (DROSOPHILA) | ENY2 | −1.94151 | | −2.25661 |
| 2034 | ENDOTHELIAL PAS DOMAIN PROTEIN 1 | EPAS1 | −2.32691 | −3.32114 | −2.88588 |
| 54566 | ERYTHROCYTE MEMBRANE PROTEIN BAND 4.1 LIKE 4B | EPB41L4B | −2.20157 | | −3.22323 |
| 2044 | EPH RECEPTOR A5 | EPHA5 | −4.13072 | −4.98528 | −5.66157 |
| 55040 | EPSIN 3 | EPN3 | −2.50122 | | −2.18479 |
| 2057 | ERYTHROPOIETIN RECEPTOR | EPOR | −1.96407 | −2.89036 | −2.10795 |
| 2067 | EXCISION REPAIR CROSS-COMPLEMENTING RODENT REPAIR | ERCC1 | −3.30242 | −3.35171 | |
| | DEFICIENCY, COMPLEMENTATION GROUP 1 (INCLUDES OVERLAPPING | | | | |
| | ANTISENSE SEQUENCE) | | | | |
| 2068 | EXCISION REPAIR CROSS-COMPLEMENTING RODENT REPAIR | ERCC2 | | −2.87085 | −3.83979 |
| | DEFICIENCY, COMPLEMENTATION GROUP 2 (XERODERMA | | | | |
| | PIGMENTOSUM D) | | | | |
| 2081 | ENDOPLASMIC RETICULUM TO NUCLEUS SIGNALLING 1 | ERN1 | −3.26923 | −2.48179 | |
| 345062 | EPIDERMIS-SPECIFIC SERINE PROTEASE-LIKE PROTEIN | ESSPL | −2.38312 | −2.17637 | |
| 2128 | EVE, EVEN-SKIPPED HOMEOBOX HOMOLOG 1 (DROSOPHILA) | EVX1 | | −2.94372 | −3.14417 |
| 2153 | COAGULATION FACTOR V (PROACCELERIN, LABILE FACTOR) | F5 | | −3.25947 | −3.02546 |
| 2172 | FATTY ACID BINDING PROTEIN 6, ILEAL (GASTROTROPIN) | FABP6 | −4.08486 | −3.59984 | −1.94519 |
| 3992 | FATTY ACID DESATURASE 1 | FADS1 | −4.03858 | −3.83463 | |
| 151313 | HYPOTHETICAL PROTEIN DKFZP434N062 | FAHD2B | −3.11705 | −3.22452 | −2.59929 |
| 9747 | KIAA0738 GENE PRODUCT | FAM115A | −3.84046 | −2.7195 | −2.45971 |
| 81558 | C/EBP-INDUCED PROTEIN | FAM117A | | −4.43236 | −2.04035 |
| 54855 | FAMILY WITH SEQUENCE SIMILARITY 46, MEMBER C | FAM46C | −2.81984 | | −2.89087 |
| 442444 | SIMILAR TO HYPOTHETICAL PROTEIN FLJ35782 | FAM47C | −2.36742 | −2.31443 | −1.86826 |
| 113115 | FAMILY WITH SEQUENCE SIMILARITY 54, MEMBER A | FAM54A | −2.3527 | −1.94078 | |
| 91775 | FAMILY WITH SEQUENCE SIMILARITY 55, MEMBER C | FAM55C | | −1.90321 | −1.90662 |
| 149297 | FAMILY WITH SEQUENCE SIMILARITY 78, MEMBER B | FAM78B | | −5.19784 | −2.27141 |
| 2177 | FANCONI ANEMIA, COMPLEMENTATION GROUP D2 | FANCD2 | −2.59596 | | −2.19731 |
| 2191 | FIBROBLAST ACTIVATION PROTEIN, ALPHA | FAP | −1.90529 | −2.09759 | |
| 10160 | FERM, RHOGEF (ARHGEF) AND PLECKSTRIN DOMAIN PROTEIN 1 | FARP1 | −2.25154 | −2.07884 | |
| | (CHONDROCYTE-DERIVED) | | | | |
| 2196 | FAT TUMOR SUPPRESSOR HOMOLOG 2 (DROSOPHILA) | FAT2 | −1.92073 | −2.64341 | |
| 54751 | FILAMIN BINDING LIM PROTEIN 1 | FBLIM1 | −2.35839 | | −2.6936 |
| 129804 | HYPOTHETICAL PROTEIN FLJ37440 | FBLN7 | | −2.02407 | −2.19875 |
| 22992 | F-BOX AND LEUCINE-RICH REPEAT PROTEIN 11 | FBXL11 | | −2.33502 | −3.60892 |
| 54620 | F-BOX AND LEUCINE-RICH REPEAT PROTEIN 19 | FBXL19 | −2.29513 | | −2.27125 |
| 126433 | F-BOX PROTEIN 27 | FBXO27 | | −2.11258 | −2.49207 |
| 26259 | F-BOX AND WD-40 DOMAIN PROTEIN 8 | FBXW8 | −2.80401 | | −2.13207 |
| 83953 | FC RECEPTOR, IGA, IGM, HIGH AFFINITY | FCAMR | −4.09839 | −1.9096 | −2.07656 |
| 9103 | FC FRAGMENT OF IGG, LOW AFFINITY IIC, RECEPTOR FOR (CD32) | FCGR2C | | −2.48481 | −2.28736 |
| 2865 | FREE FATTY ACID RECEPTOR 3 | FFAR3 | −3.5176 | | −2.17937 |
| 9457 | FOUR AND A HALF LIM DOMAINS 5 | FHL5 | −3.72762 | −2.80553 | |
| 2307 | FORKHEAD-LIKE 18 (DROSOPHILA) | FKHL18 | −2.99411 | −3.40374 | |
| 388939 | SIMILAR TO CDNA SEQUENCE BC027072 | FLJ34931 | −3.78588 | −2.21798 | |
| 222183 | HYPOTHETICAL PROTEIN FLJ37078 | FLJ37078 | −2.99604 | | −2.8664 |
| 643853 | SIMILAR TO F40B5.2B | FLJ45032 | −2.2066 | −2.91152 | |
| 440107 | FLJ46688 PROTEIN | FLJ46688 | −2.94624 | −2.03724 | |
| 23769 | FIBRONECTIN LEUCINE RICH TRANSMEMBRANE PROTEIN 1 | FLRT1 | −3.78841 | −2.82884 | |
| 2324 | FMS-RELATED TYROSINE KINASE 4 | FLT4 | −1.93582 | −2.52564 | |
| 2348 | FOLATE RECEPTOR 1 (ADULT) | FOLR1 | −1.87156 | −4.52012 | |
| 2350 | FOLATE RECEPTOR 2 (FETAL) | FOLR2 | −2.22889 | −3.43517 | |
| 442425 | SIMILAR TO FOXB2 PROTEIN | FOXB2 | −3.4805 | −2.17358 | |
| 22887 | FORKHEAD BOX J3 | FOXJ3 | | −3.34354 | −1.97325 |
| 93986 | TRINUCLEOTIDE REPEAT CONTAINING 10 | FOXP2 | −3.44945 | −2.52173 | −2.81991 |
| 2487 | FRIZZLED-RELATED PROTEIN | FRZB | | −2.18499 | −1.93131 |
| 2492 | FOLLICLE STIMULATING HORMONE RECEPTOR | FSHR | −3.11704 | −2.88641 | −3.92828 |
| 10468 | FOLLISTATIN | FST | −4.64815 | −2.97286 | −4.3585 |
| 2528 | FUCOSYLTRANSFERASE 6 (ALPHA (1,3) FUCOSYLTRANSFERASE) | FUT6 | | −2.28549 | −2.97626 |
| 2533 | FYN BINDING PROTEIN (FYB-120/130) | FYB | −1.97259 | | −2.54165 |
| 2535 | FRIZZLED HOMOLOG 2 (DROSOPHILA) | FZD2 | −1.89392 | | −2.92839 |
| 139716 | GRB2-ASSOCIATED BINDING PROTEIN 3 | GAB3 | | −4.11116 | −2.31614 |
| 2562 | GAMMA-AMINOBUTYRIC ACID (GABA) A RECEPTOR, BETA 3 | GABRB3 | −2.5385 | −2.00785 | |
| 130589 | GALACTOSE MUTAROTASE (ALDOSE 1-EPIMERASE) | GALM | | −2.21926 | −2.53267 |
| 2588 | GALACTOSAMINE (N-ACETYL)-6-SULFATE SULFATASE (MORQUIO | GALNS | −2.64464 | | −1.87302 |
| | SYNDROME, MUCOPOLYSACCHARIDOSIS TYPE IVA) | | | | |
| 51809 | UDP-N-ACETYL-ALPHA-D-GALACTOSAMINE:POLYPEPTIDE N- | GALNT7 | | −2.28686 | −2.24023 |
| | ACETYLGALACTOSAMINYLTRANSFERASE 7 (GALNAC-T7) | | | | |
| 117248 | UDP-N-ACETYL-ALPHA-D-GALACTOSAMINE:POLYPEPTIDE N- | GALNTL2 | −3.01111 | −2.98456 | −3.25224 |
| | ACETYLGALACTOSAMINYLTRANSFERASE-LIKE 2 | | | | |
| 8522 | GROWTH ARREST-SPECIFIC 7 | GAS7 | −2.53612 | −2.54686 | −3.13161 |
| 115361 | GUANYLATE BINDING PROTEIN 4 | GBP4 | −2.68608 | −2.62916 | |
| 9247 | GLIAL CELLS MISSING HOMOLOG 2 (DROSOPHILA) | GCM2 | −4.05623 | −1.99879 | |
| 151449 | GROWTH DIFFERENTIATION FACTOR 7 | GDF7 | −3.36855 | −1.93765 | −2.16948 |
| 2664 | GDP DISSOCIATION INHIBITOR 1 | GDI1 | −2.87815 | −3.44471 | −3.453 |
| 199720 | GAMETOGENETIN | GGN | −3.75431 | −2.10603 | −3.11222 |
| 2693 | GROWTH HORMONE SECRETAGOGUE RECEPTOR | GHSR | −1.87572 | −2.68113 | |
| 54826 | HYPOTHETICAL PROTEIN FLJ20125 | GIN1 | −3.47563 | −3.9474 | |
| 169792 | GLIS FAMILY ZINC FINGER 3 | GLIS3 | −2.8858 | −2.03553 | −1.9006 |
| 9340 | GLUCAGON-LIKE PEPTIDE 2 RECEPTOR | GLP2R | −2.76923 | −3.31118 | −2.45118 |
| 2752 | GLUTAMATE-AMMONIA LIGASE (GLUTAMINE SYNTHETASE) | GLUL | −1.9631 | | −1.86752 |
| 2769 | GUANINE NUCLEOTIDE BINDING PROTEIN (G PROTEIN), ALPHA 15 (GQ | GNA15 | −2.40069 | −2.58242 | |
| | CLASS) | | | | |
| 2778 | GNAS COMPLEX LOCUS | GNAS | −3.95239 | −3.13278 | −3.19362 |
| 2781 | GUANINE NUCLEOTIDE BINDING PROTEIN (G PROTEIN), ALPHA Z | GNAZ | −4.24008 | −2.58879 | −3.72885 |
| | POLYPEPTIDE | | | | |
| 2787 | GUANINE NUCLEOTIDE BINDING PROTEIN (G PROTEIN), GAMMA 5 | GNG5 | | −3.99978 | −3.03387 |
| 2794 | GUANINE NUCLEOTIDE BINDING PROTEIN-LIKE 1 | GNL1 | −3.26378 | | −2.57438 |
| 55638 | HYPOTHETICAL PROTEIN FLJ20366 | GOLSYN | −1.91757 | | −2.45471 |
| 54856 | GON-4-LIKE (C. ELEGANS) | GON4L | −2.56712 | −3.21962 | |
| 64689 | GOLGI REASSEMBLY STACKING PROTEIN 1, 65 KDA | GORASP1 | −2.65971 | −3.85635 | |
| 8733 | GLYCOSYLPHOSPHATIDYLINOSITOL ANCHOR ATTACHMENT PROTEIN 1 | GPAA1 | −4.74693 | −2.20232 | |
| | HOMOLOG (YEAST) | | | | |
| 2239 | GLYPICAN 4 | GPC4 | | −3.67898 | −2.66778 |
| 56927 | G PROTEIN-COUPLED RECEPTOR 108 | GPR108 | −3.65187 | −1.91374 | |
| 266977 | HYPOTHETICAL PROTEIN FLJ22684 | GPR110 | −3.10349 | −3.28571 | |
| 283383 | G PROTEIN-COUPLED RECEPTOR 133 | GPR133 | −2.62395 | | −1.87835 |
| 124274 | G PROTEIN-COUPLED RECEPTOR 139 | GPR139 | −2.32763 | | −3.7658 |
| 353345 | G PROTEIN-COUPLED RECEPTOR 141 | GPR141 | −3.53403 | −1.94583 | |
| 4935 | G PROTEIN-COUPLED RECEPTOR 143 | GPR143 | | −1.98133 | −2.81368 |
| 57512 | G PROTEIN-COUPLED RECEPTOR 158 | GPR158 | −4.59117 | −2.00912 | |
| 79581 | G PROTEIN-COUPLED RECEPTOR 172A | GPR172A | −2.67151 | | −1.9873 |
| 2866 | G PROTEIN-COUPLED RECEPTOR 42 | GPR42 | −3.58878 | −2.52295 | −2.31642 |
| 10149 | G PROTEIN-COUPLED RECEPTOR 64 | GPR64 | −2.88228 | −2.33642 | |
| 8111 | G PROTEIN-COUPLED RECEPTOR 68 | GPR68 | −4.6542 | | −2.79728 |
| 114928 | G PROTEIN-COUPLED RECEPTOR ASSOCIATED SORTING PROTEIN 2 | GPRASP2 | −2.59236 | | −2.06134 |
| 26086 | G-PROTEIN SIGNALLING MODULATOR 1 (AGS3-LIKE, C. ELEGANS) | GPSM1 | −2.96143 | −2.5368 | |
| 23708 | G1 TO S PHASE TRANSITION 2 | GSPT2 | −2.08666 | −2.49344 | |
| 79807 | HYPOTHETICAL PROTEIN FLJ13273 | GSTCD | −1.94122 | −1.85446 | |
| 9328 | GENERAL TRANSCRIPTION FACTOR IIIC, POLYPEPTIDE 5, 63 KDA | GTF3C5 | −3.29436 | −2.28714 | −2.33891 |
| 474382 | H2A HISTONE FAMILY, MEMBER B3 | H2AFB3 | −3.11566 | | −2.0306 |
| 3066 | HISTONE DEACETYLASE 2 | HDAC2 | −1.85138 | −3.34838 | −2.95007 |
| 8841 | HISTONE DEACETYLASE 3 | HDAC3 | −2.55243 | | −2.05805 |
| 3067 | HISTIDINE DECARBOXYLASE | HDC | −2.17027 | | −2.3297 |
| 25831 | HECT DOMAIN CONTAINING 1 | HECTD1 | −2.9087 | | −1.91184 |
| 57520 | HECT, C2 AND WW DOMAIN CONTAINING E3 UBIQUITIN PROTEIN | HECW2 | −4.96002 | −2.65557 | |
| | LIGASE 2 | | | | |
| 220296 | HEPATOCYTE CELL ADHESION MOLECULE | HEPN1 | −2.35488 | −2.28112 | |
| 64399 | HEDGEHOG INTERACTING PROTEIN | HHIP | −3.09954 | −2.78448 | |
| 3090 | HYPERMETHYLATED IN CANCER 1 | HIC1 | −5.18038 | −4.25796 | −2.45575 |
| 192286 | HIG1 DOMAIN FAMILY, MEMBER 2A | HIGD2A | −2.01104 | −3.8801 | |
| 8342 | HISTONE 1, H2BM | HIST1H2BM | −3.01447 | | −2.34978 |
| 8352 | HISTONE 1, H3A | HIST1H3J | −2.26906 | −2.2589 | −2.45454 |
| 3118 | MAJOR HISTOCOMPATIBILITY COMPLEX, CLASS II, DQ ALPHA 1 | HLA- | −2.70344 | −1.96681 | |
| | | DQA2 | | | |
| 3127 | MAJOR HISTOCOMPATIBILITY COMPLEX, CLASS II, DR BETA 1 | HLA- | −1.96407 | −3.55599 | |
| | | DRB5 | | | |
| 3145 | HYDROXYMETHYLBILANE SYNTHASE | HMBS | −2.61436 | −2.74885 | −1.98454 |
| 10473 | HIGH MOBILITY GROUP NUCLEOSOMAL BINDING DOMAIN 4 | HMGN4 | −1.85907 | −2.09832 | |
| 10949 | HETEROGENEOUS NUCLEAR RIBONUCLEOPROTEIN A0 | HNRNPA0 | | −2.18951 | −2.89115 |
| 9455 | HOMER HOMOLOG 2 (DROSOPHILA) | HOMER2 | −3.9976 | | −2.6051 |
| 3206 | HOMEOBOX A10 | HOXA10 | −1.86466 | −3.79496 | −3.24011 |
| 3219 | HOMEOBOX B9 | HOXB9 | | −2.23595 | −2.86036 |
| 3238 | HOMEOBOX D12 | HOXD12 | −3.21087 | −2.52466 | |
| 3248 | HYDROXYPROSTAGLANDIN DEHYDROGENASE 15-(NAD) | HPGD | −1.86577 | | −2.55677 |
| 54979 | HRAS-LIKE SUPPRESSOR 2 | HRASLS2 | −3.15182 | −1.89794 | |
| 117245 | HRAS-LIKE SUPPRESSOR FAMILY, MEMBER 5 | HRASLS5 | −3.33291 | −3.60836 | |
| 3273 | HISTIDINE-RICH GLYCOPROTEIN | HRG | −3.18723 | −3.4721 | −2.06195 |
| 64342 | HS1-BINDING PROTEIN 3 | HS1BP3 | −2.96461 | −3.11986 | −2.4583 |
| 90161 | HEPARAN SULFATE 6-O-SULFOTRANSFERASE 2 | HS6ST2 | | −2.77556 | −2.84445 |
| 345275 | HYDROXYSTEROID (17-BETA) DEHYDROGENASE 13 | HSD17B13 | −2.00983 | −2.12285 | |
| 3294 | HYDROXYSTEROID (17-BETA) DEHYDROGENASE 2 | HSD17B2 | −4.49553 | −3.43069 | |
| 8630 | HYDROXYSTEROID (17-BETA) DEHYDROGENASE 6 | HSD17B6 | −3.46952 | −2.23407 | −3.31334 |
| 3356 | 5-HYDROXYTRYPTAMINE (SEROTONIN) RECEPTOR 2A | HTR2A | −2.29234 | | −3.52702 |
| 23463 | ISOPRENYLCYSTEINE CARBOXYL METHYLTRANSFERASE | ICMT | −3.03314 | −2.30793 | |
| 51278 | IMMEDIATE EARLY RESPONSE 5 | IER5 | −1.91326 | −2.78467 | |
| 439996 | INTERFERON-INDUCED PROTEIN WITH TETRATRICOPEPTIDE REPEATS | IFIT1L | −2.40962 | | −2.43055 |
| | 1-LIKE | | | | |
| 3446 | INTERFERON, ALPHA 10 | IFNA10 | −5.85688 | −3.18894 | |
| 3456 | INTERFERON, BETA 1, FIBROBLAST | IFNB1 | −2.97838 | −2.04472 | |
| 26160 | INTRAFLAGELLAR TRANSPORT 172 HOMOLOG (CHLAMYDOMONAS) | IFT172 | −2.03712 | −2.47326 | |
| 3488 | INSULIN-LIKE GROWTH FACTOR BINDING PROTEIN 5 | IGFBP5 | | −2.5889 | −2.46803 |
| 3489 | INSULIN-LIKE GROWTH FACTOR BINDING PROTEIN 6 | IGFBP6 | | −1.98094 | −2.28982 |
| 9641 | INHIBITOR OF KAPPA LIGHT POLYPEPTIDE GENE ENHANCER IN B- | IKBKE | −2.71769 | −4.20239 | |
| | CELLS, KINASE EPSILON | | | | |
| 64375 | ZINC FINGER PROTEIN, SUBFAMILY 1A, 4 (EOS) | IKZF4 | −4.50915 | −3.62475 | |
| 53342 | INTERLEUKIN 17D | IL17D | −2.57159 | −2.31667 | |
| 53832 | INTERLEUKIN 20 RECEPTOR, ALPHA | IL20RA | −2.19032 | −2.76022 | |
| 9235 | INTERLEUKIN 32 | IL32 | −2.10552 | | −2.01423 |
| 3608 | INTERLEUKIN ENHANCER BINDING FACTOR 2, 45 KDA | ILF2 | −3.12856 | −2.40931 | |
| 55272 | IMP3, U3 SMALL NUCLEOLAR RIBONUCLEOPROTEIN, HOMOLOG | IMP3 | | −1.9611 | −2.08666 |
| | (YEAST) | | | | |
| 440068 | INHIBITORY CASPASE RECRUITMENT DOMAIN (CARD) PROTEIN | INCA | −3.61419 | −2.14377 | |
| 10022 | INSULIN-LIKE 5 | INSL5 | −1.85932 | | −2.36095 |
| 3642 | INSULINOMA-ASSOCIATED 1 | INSM1 | −4.57994 | −4.7159 | −2.96116 |
| 26512 | DKFZP434B105 PROTEIN | INTS6 | −2.46922 | −3.1278 | |
| 79711 | IMPORTIN 4 | IPO4 | −2.19665 | −2.88652 | −3.89452 |
| 3656 | INTERLEUKIN-1 RECEPTOR-ASSOCIATED KINASE 2 | IRAK2 | −5.14744 | −2.90272 | −4.4479 |
| 10379 | INTERFERON-STIMULATED TRANSCRIPTION FACTOR 3, GAMMA 48 KDA | IRF9 | −3.29863 | −5.05236 | −2.76033 |
| 8471 | INSULIN RECEPTOR SUBSTRATE 4 | IRS4 | −4.12096 | −3.11419 | |
| 57611 | IMMUNOGLOBULIN SUPERFAMILY CONTAINING LEUCINE-RICH REPEAT 2 | ISLR2 | | −2.67151 | −2.3868 |
| 8516 | INTEGRIN, ALPHA 8 | ITGA8 | −4.66407 | −2.77516 | |
| 3698 | INTER-ALPHA (GLOBULIN) INHIBITOR H2 | ITIH2 | −1.92876 | −3.06384 | |
| 3712 | ISOVALERYL COENZYME A DEHYDROGENASE | IVD | | −1.97496 | −2.22619 |
| 23081 | JUMONJI DOMAIN CONTAINING 2C | JMJD2C | −3.5406 | −2.84532 | −3.65305 |
| 56704 | JUNCTOPHILIN 1 | JPH1 | −5.13892 | −3.00751 | −4.09157 |
| 10899 | JUMPING TRANSLOCATION BREAKPOINT | JTB | | −1.92897 | −2.2368 |
| 27133 | POTASSIUM VOLTAGE-GATED CHANNEL, SUBFAMILY H (EAG- | KCNH5 | −2.18888 | | −2.30075 |
| | RELATED), MEMBER 5 | | | | |
| 9798 | KIAA0174 | KIAA0174 | −6.04739 | −2.65805 | |
| 9895 | KIAA0329 | KIAA0329 | −2.72568 | −3.29382 | |
| 23334 | KIAA0467 | KIAA0467 | −2.73811 | | −1.89679 |
| 9858 | KIAA0649 | KIAA0649 | −2.87843 | | −2.07629 |
| 57521 | RAPTOR | KIAA1303 | −2.31287 | −2.90653 | −2.4201 |
| 57650 | KIAA1524 | KIAA1524 | −3.72788 | −2.34109 | |
| 80817 | KIAA1712 | KIAA1712 | | −3.59888 | −2.5921 |
| 85449 | KIAA1755 PROTEIN | KIAA1755 | −3.70085 | −4.56373 | −3.39759 |
| 90231 | KIAA2013 | KIAA2013 | −2.09224 | −2.6924 | |
| 57576 | KINESIN FAMILY MEMBER 17 | KIF17 | | −2.57894 | −2.97311 |
| 124602 | KINESIN FAMILY MEMBER 19 | KIF19 | −2.11698 | −2.1023 | −3.33649 |
| 9493 | KINESIN FAMILY MEMBER 23 | KIF23 | −2.05 | −1.96415 | |
| 26153 | KINESIN FAMILY MEMBER 26A | KIF26A | −2.31143 | | −2.35145 |
| 11278 | KRUPPEL-LIKE FACTOR 12 | KLF12 | −1.862 | −2.25234 | |
| 23588 | KELCH DOMAIN CONTAINING 2 | KLHDC2 | −3.17501 | −3.05664 | |
| 56062 | KELCH (DROSOPHILA)-LIKE 4 | KLHL4 | −2.93844 | −2.56313 | |
| 9622 | KALLIKREIN 4 (PROSTASE, ENAMEL MATRIX, PROSTATE) | KLK4 | | −3.51019 | −2.13427 |
| 353323 | KERATIN ASSOCIATED PROTEIN 12-2 | KRTAP12-2 | | −4.07607 | −4.27747 |
| 337972 | KERATIN ASSOCIATED PROTEIN 19-5 | KRTAP19-5 | −2.10694 | −1.86912 | −1.8969 |
| 85287 | KERATIN ASSOCIATED PROTEIN 4-7 | KRTAP4-7 | −3.1366 | −3.19935 | |
| 440023 | KERATIN ASSOCIATED PROTEIN 5-6 | KRTAP5-6 | | −2.64506 | −2.60634 |
| 388533 | KIPV467 | KRTDAP | −3.84172 | −3.12836 | |
| 56983 | CHROMOSOME 3 OPEN READING FRAME 9 | KTELC1 | −2.64341 | −3.73793 | |
| 84456 | L(3)MBT-LIKE 3 (DROSOPHILA) | L3MBTL3 | | −2.78776 | −2.27559 |
| 3916 | LYSOSOMAL-ASSOCIATED MEMBRANE PROTEIN 1 | LAMP1 | | −2.02168 | −2.60901 |
| 143903 | LAYILIN | LAYN | | −2.46796 | −2.73265 |
| 3930 | LAMIN B RECEPTOR | LBR | −4.19709 | −1.91704 | |
| 85474 | LADYBIRD HOMEOBOX HOMOLOG 2 (DROSOPHILA) | LBX2 | | −4.38027 | −2.79667 |
| 353139 | LATE CORNIFIED ENVELOPE 2A | LCE2A | −2.31879 | −2.94629 | |
| 84458 | LIGAND-DEPENDENT COREPRESSOR | LCOR | | −3.16053 | −2.74306 |
| 11061 | LEUKOCYTE CELL DERIVED CHEMOTAXIN 1 | LECT1 | −2.71543 | −3.92097 | |
| 3965 | LECTIN, GALACTOSIDE-BINDING, SOLUBLE, 9 (GALECTIN 9) | LGALS9 | −2.1954 | −1.90199 | |
| 10186 | LIPOMA HMGIC FUSION PARTNER | LHFP | −2.00938 | −2.83484 | |
| 375612 | LIPOMA HMGIC FUSION PARTNER-LIKE 3 | LHFPL3 | −1.90933 | −2.88839 | |
| 375323 | LIPOMA HMGIC FUSION PARTNER-LIKE PROTEIN 4 | LHFPL4 | −1.96573 | | −2.02048 |
| 3985 | LIM DOMAIN KINASE 2 | LIMK2 | | −2.40384 | −2.33116 |
| 96626 | LIM AND SENESCENT CELL ANTIGEN-LIKE DOMAINS 3 | LIMS3 | −3.87705 | −2.19621 | −2.81259 |
| 64130 | LIN-7 HOMOLOG B (C. ELEGANS) | LIN7B | −4.05261 | | −3.26445 |
| 158038 | LEUCINE RICH REPEAT NEURONAL 6C | LINGO2 | −2.22343 | −1.8526 | −2.0549 |
| 84823 | LAMIN B2 | LMNB2 | −3.57509 | −2.2521 | |
| 8543 | LIM DOMAIN ONLY 4 | LMO4 | −2.89404 | −2.082 | −2.42817 |
| 348801 | HYPOTHETICAL PROTEIN LOC348801 | LNP1 | | −2.25674 | −2.73949 |
| 126075 | HYPOTHETICAL PROTEIN LOC126075 | LOC126075 | −2.45589 | | −2.93435 |
| 153364 | SIMILAR TO METALLO-BETA-LACTAMASE SUPERFAMILY PROTEIN | LOC153364 | −1.95863 | −4.24838 | −3.71792 |
| 161247 | SIMILAR TO CG10671-LIKE | LOC161247 | −3.371 | −2.40455 | |
| 162993 | HYPOTHETICAL PROTEIN LOC162993 | LOC162993 | −2.16393 | −3.31765 | −1.97896 |
| 201725 | HYPOTHETICAL PROTEIN LOC201725 | LOC201725 | −5.68546 | | −3.94516 |
| 202459 | SIMILAR TO RIKEN CDNA 2310008M10 | LOC202459 | | −1.94148 | −3.3487 |
| 26010 | DNA POLYMERASE-TRANSACTIVATED PROTEIN 6 | LOC26010 | −1.96021 | −2.25198 | −2.23318 |
| 283677 | HYPOTHETICAL LOC283677 | LOC283677 | | −2.64581 | −4.19504 |
| 338809 | HYPOTHETICAL PROTEIN LOC338809 | LOC338809 | −3.81319 | −1.94045 | |
| 390243 | SIMILAR TO FOLATE RECEPTOR 4 (DELTA) ISOFORM 1 | LOC390243 | −2.36576 | −2.36707 | −1.89087 |
| 399818 | SIMILAR TO CG9643-PA | LOC399818 | −1.99734 | −2.05135 | |
| 400506 | SIMILAR TO TSG118.1 | LOC400506 | −5.33979 | | −2.27212 |
| 440093 | SIMILAR TO H3 HISTONE, FAMILY 3B | LOC440093 | −2.02969 | −1.88758 | −3.3132 |
| 441294 | SIMILAR TO CTAGE6 | LOC441294 | −2.26952 | | −2.2475 |
| 51057 | HYPOTHETICAL PROTEIN LOC51057 | LOC51057 | −1.8843 | | −2.3085 |
| 63920 | TRANSPOSON-DERIVED BUSTER3 TRANSPOSASE-LIKE | LOC63920 | −3.94367 | −2.89712 | |
| 643905 | SIMILAR TO PROTOCADHERIN 15B | LOC643905 | −1.93464 | −2.62212 | |
| 653192 | SIMILAR TO TRIPARTITE MOTIF PROTEIN 17 | LOC653192 | −1.98189 | | −3.01251 |
| 653319 | SIMILAR TO HYPOTHETICAL PROTEIN LOC283849 | LOC653319 | −2.95378 | | −2.33583 |
| 90835 | HYPOTHETICAL PROTEIN LOC90835 | LOC90835 | | −2.72483 | −3.6019 |
| 84171 | LYSYL OXIDASE-LIKE 4 | LOXL4 | | −2.73286 | −2.93429 |
| 9663 | LIPIN 2 | LPIN2 | | −2.54186 | −3.12008 |
| 79782 | LEUCINE RICH REPEAT CONTAINING 31 | LRRC31 | −2.86285 | −1.98006 | −2.17554 |
| 64101 | LEUCINE RICH REPEAT CONTAINING 4 | LRRC4 | −2.62687 | −2.28397 | |
| 94030 | LEUCINE RICH REPEAT CONTAINING 4B | LRRC4B | | −3.66397 | −3.0838 |
| 220074 | LEUCINE RICH REPEAT CONTAINING 51 | LRRC51 | −2.13492 | −2.49709 | |
| 9209 | LEUCINE RICH REPEAT (IN FLII) INTERACTING PROTEIN 2 | LRRFIP2 | −2.61026 | −2.36935 | |
| 338821 | ORGANIC ANION TRANSPORTER LST-3B | LST- | | −2.46569 | −1.88141 |
| | | 3TM12 | | | |
| 51213 | LEUCINE ZIPPER PROTEIN 4 | LUZP4 | −2.34327 | −3.27072 | |
| 27076 | LY6/PLAUR DOMAIN CONTAINING 3 | LYPD3 | −3.6252 | −2.24353 | −2.00614 |
| 130574 | HYPOTHETICAL PROTEIN MGC52057 | LYPD6 | −3.48063 | −4.26156 | |
| 84445 | LEUCINE ZIPPER, PUTATIVE TUMOR SUPPRESSOR 2 | LZTS2 | −3.7582 | −2.00544 | |
| 4081 | MAB-21-LIKE 1 (C. ELEGANS) | MAB21L1 | −4.44621 | −3.48347 | −2.45736 |
| 84944 | MAELSTROM HOMOLOG (DROSOPHILA) | MAEL | −3.34187 | | −2.07536 |
| 9935 | V-MAF MUSCULOAPONEUROTIC FIBROSARCOMA ONCOGENE | MAFB | | −2.01537 | −1.96292 |
| | HOMOLOG B (AVIAN) | | | | |
| 4113 | MELANOMA ANTIGEN FAMILY B, 2 | MAGEB2 | −1.9079 | | −2.59269 |
| 5607 | MITOGEN-ACTIVATED PROTEIN KINASE KINASE 5 | MAP2K5 | −3.44358 | | −2.68431 |
| 9064 | MITOGEN-ACTIVATED PROTEIN KINASE KINASE KINASE 6 | MAP3K6 | −3.60211 | −2.69142 | |
| 6885 | MITOGEN-ACTIVATED PROTEIN KINASE KINASE KINASE 7 | MAP3K7 | −4.37075 | −2.60108 | −3.08528 |
| 54799 | MBT DOMAIN CONTAINING 1 | MBTD1 | | −2.01418 | −2.29198 |
| 92014 | MITOCHONDRIAL CARRIER TRIPLE REPEAT 1 | MCART1 | −2.73059 | −3.29002 | −2.95126 |
| 4172 | MCM3 MINICHROMOSOME MAINTENANCE DEFICIENT 3 (S. CEREVISIAE) | MCM3 | −2.49008 | −3.67504 | |
| 28985 | MALIGNANT T CELL AMPLIFIED SEQUENCE 1 | MCTS1 | −1.96517 | | −2.46353 |
| 9656 | MEDIATOR OF DNA DAMAGE CHECKPOINT 1 | MDC1 | −2.65123 | | −2.46605 |
| 4197 | ECOTROPIC VIRAL INTEGRATION SITE 1 | MDS1 | −4.91817 | −4.36995 | −3.49786 |
| 400569 | SIMILAR TO HSPC296 | MED11 | −2.33766 | | −1.89359 |
| 80306 | MEDIATOR OF RNA POLYMERASE II TRANSCRIPTION, SUBUNIT 28 | MED28 | | −2.33833 | −2.31198 |
| | HOMOLOG (YEAST) | | | | |
| 51003 | MEDIATOR OF RNA POLYMERASE II TRANSCRIPTION, SUBUNIT 31 | MED31 | −2.04877 | −3.62991 | |
| | HOMOLOG (YEAST) | | | | |
| 4207 | MADS BOX TRANSCRIPTION ENHANCER FACTOR 2, POLYPEPTIDE B | MEF2B | −5.35307 | −4.30158 | −1.89447 |
| | (MYOCYTE ENHANCER FACTOR 2B) | | | | |
| 1954 | EGF-LIKE-DOMAIN, MULTIPLE 4 | MEGF8 | −2.25397 | −2.14012 | −3.97425 |
| 64747 | MAJOR FACILITATOR SUPERFAMILY DOMAIN CONTAINING 1 | MFSD1 | −3.55784 | | −2.97033 |
| 84804 | HYPOTHETICAL PROTEIN MGC11332 | MFSD9 | −2.2117 | | −2.04748 |
| 80772 | HYPOTHETICAL PROTEIN MGC10334 | MGC10334 | −3.15307 | | −2.81389 |
| 92806 | HYPOTHETICAL PROTEIN MGC13198 | MGC16385 | −3.67807 | −2.73927 | −5.34412 |
| 167359 | HYPOTHETICAL PROTEIN MGC42105 | MGC42105 | −2.13288 | −2.55735 | −4.37954 |
| 401145 | SIMILAR TO KIAA1680 PROTEIN | MGC48628 | −2.65392 | −1.9292 | |
| 4259 | MICROSOMAL GLUTATHIONE S-TRANSFERASE 3 | MGST3 | −3.23003 | −2.59358 | −2.15506 |
| 166968 | HYPOTHETICAL PROTEIN | MIER3 | −1.948 | −2.33645 | −2.37991 |
| 4323 | MATRIX METALLOPEPTIDASE 14 (MEMBRANE-INSERTED) | MMP14 | | −2.50866 | −2.79277 |
| 283385 | MORN REPEAT CONTAINING 3 | MORN3 | −1.89433 | | −2.20862 |
| 758 | METALLOPHOSPHOESTERASE DOMAIN CONTAINING 1 | MPPED1 | | −2.74881 | −1.94218 |
| 4360 | MANNOSE RECEPTOR, C TYPE 1 | MRC1 | −2.64776 | | −3.15523 |
| 4361 | MRE11 MEIOTIC RECOMBINATION 11 HOMOLOG A (S. CEREVISIAE) | MRE11A | −4.30835 | | −3.5375 |
| 64981 | MITOCHONDRIAL RIBOSOMAL PROTEIN L34 | MRPL34 | −2.75412 | −4.96395 | |
| 341116 | MEMBRANE-SPANNING 4-DOMAINS, SUBFAMILY A, MEMBER 10 | MS4A10 | −2.65751 | −2.93441 | −3.4722 |
| 4477 | MICROSEMINOPROTEIN, BETA- | MSMB | | −2.88215 | −2.46353 |
| 4504 | METALLOTHIONEIN 3 (GROWTH INHIBITORY FACTOR | MT3 | −4.04357 | −2.8508 | |
| | (NEUROTROPHIC)) | | | | |
| 8776 | MYOTUBULARIN RELATED PROTEIN 1 | MTMR1 | −4.38585 | −1.85331 | −2.20333 |
| 136319 | MYOTROPHIN | MTPN | −4.08597 | −4.42856 | −2.97718 |
| 4547 | MICROSOMAL TRIGLYCERIDE TRANSFER PROTEIN | MTTP | −2.50767 | −2.26107 | −4.23045 |
| 57509 | MITOCHONDRIAL TUMOR SUPPRESSOR 1 | MTUS1 | | −3.12976 | −2.58471 |
| 143662 | MUCIN 15 | MUC15 | −2.19721 | −2.12986 | |
| 4589 | MUCIN 7, SALIVARY | MUC7 | −3.0859 | −2.50545 | −3.22334 |
| 4599 | MYXOVIRUS (INFLUENZA VIRUS) RESISTANCE 1, INTERFERON- | MX1 | −2.36526 | −2.01844 | −2.50688 |
| | INDUCIBLE PROTEIN P78 (MOUSE) | | | | |
| 343263 | MYOSIN BINDING PROTEIN H-LIKE | MYBPHL | −2.91997 | | −1.87908 |
| 4641 | MYOSIN IC | MYO1C | −3.75824 | −2.04836 | |
| 79829 | HYPOTHETICAL PROTEIN FLJ13848 | NAT11 | −2.49128 | −2.41016 | −1.93249 |
| 26151 | N-ACETYLTRANSFERASE 9 | NAT9 | −2.83485 | −2.2593 | |
| 284565 | NEUROBLASTOMA BREAKPOINT FAMILY, MEMBER 14 | NBPF15 | −4.33412 | −2.39879 | |
| 83988 | NEUROCALCIN DELTA | NCALD | −2.80549 | | −1.98663 |
| 4739 | NEURAL PRECURSOR CELL EXPRESSED, DEVELOPMENTALLY DOWN- | NEDD9 | −2.31196 | −3.62758 | −2.72499 |
| | REGULATED 9 | | | | |
| 4751 | NIMA (NEVER IN MITOSIS GENE A)-RELATED KINASE 2 | NEK2 | −2.9558 | −2.43274 | −2.95423 |
| 26012 | NASAL EMBRYONIC LHRH FACTOR | NELF | | −2.52297 | −2.09509 |
| 4776 | NUCLEAR FACTOR OF ACTIVATED T-CELLS, CYTOPLASMIC, | NFATC4 | −3.61264 | −3.21774 | |
| | CALCINEURIN-DEPENDENT 4 | | | | |
| 4778 | NUCLEAR FACTOR (ERYTHROID-DERIVED 2), 45 KDA | NFE2 | | −2.12929 | −2.10967 |
| 4802 | NUCLEAR TRANSCRIPTION FACTOR Y, GAMMA | NFYC | −1.91075 | −2.64669 | −1.89951 |
| 159296 | NK2 TRANSCRIPTION FACTOR HOMOLOG C (DROSOPHILA) | NKX2-3 | −3.37969 | −2.68783 | −2.46978 |
| 51701 | NEMO-LIKE KINASE | NLK | −3.33568 | −1.88129 | −2.20263 |
| 4829 | NEUROMEDIN B RECEPTOR | NMBR | −2.78007 | −3.06621 | |
| 10201 | NON-METASTATIC CELLS 6, PROTEIN EXPRESSED IN (NUCLEOSIDE- | NME6 | −2.20441 | −2.52635 | |
| | DIPHOSPHATE KINASE) | | | | |
| 129521 | NEUROMEDIN S | NMS | −5.61174 | −3.74196 | −2.58827 |
| 23530 | NICOTINAMIDE NUCLEOTIDE TRANSHYDROGENASE | NNT | −3.34428 | −3.40667 | |
| 4838 | NODAL HOMOLOG (MOUSE) | NODAL | −4.72064 | −2.31463 | |
| 27035 | NADPH OXIDASE 1 | NOX1 | −2.99102 | | −1.9221 |
| 152519 | NIPA-LIKE DOMAIN CONTAINING 1 | NPAL1 | −2.59477 | −2.24146 | |
| 190 | NUCLEAR RECEPTOR SUBFAMILY 0, GROUP B, MEMBER 1 | NR0B1 | −2.82937 | −2.00294 | |
| 4929 | NUCLEAR RECEPTOR SUBFAMILY 4, GROUP A, MEMBER 2 | NR4A2 | −4.44886 | −5.87805 | −2.9839 |
| 340371 | NUCLEAR RECEPTOR BINDING PROTEIN 2 | NRBP2 | | −2.79772 | −2.53212 |
| 4898 | NARDILYSIN (N-ARGININE DIBASIC CONVERTASE) | NRD1 | −4.28791 | −2.27774 | −2.58532 |
| 83714 | NUCLEAR RECEPTOR INTERACTING PROTEIN 2 | NRIP2 | −3.89044 | −5.23847 | −4.89818 |
| 22978 | 5′-NUCLEOTIDASE, CYTOSOLIC II | NT5C2 | | −2.93429 | −3.38901 |
| 4908 | NEUROTROPHIN 3 | NTF3 | −2.8015 | −4.06468 | |
| 4917 | NETRIN 2-LIKE (CHICKEN) | NTN2L | −3.4598 | −3.1736 | |
| 4923 | NEUROTENSIN RECEPTOR 1 (HIGH AFFINITY) | NTSR1 | −4.10473 | −3.49957 | |
| 23620 | NEUROTENSIN RECEPTOR 2 | NTSR2 | −3.90077 | −4.85068 | |
| 256281 | NUDIX (NUCLEOSIDE DIPHOSPHATE LINKED MOIETY X)-TYPE MOTIF 14 | NUDT14 | −2.33744 | −4.58128 | |
| 51203 | NUCLEOLAR AND SPINDLE ASSOCIATED PROTEIN 1 | NUSAP1 | −3.24207 | −2.43638 | |
| 56000 | NUCLEAR RNA EXPORT FACTOR 3 | NXF3 | −4.03665 | −4.20158 | −2.41973 |
| 55916 | NUCLEAR TRANSPORT FACTOR 2-LIKE EXPORT FACTOR 2 | NXT2 | −2.78623 | −2.45246 | |
| 220323 | OAF HOMOLOG (DROSOPHILA) | OAF | −3.31157 | −1.91214 | |
| 51686 | ORNITHINE DECARBOXYLASE ANTIZYME 3 | OAZ3 | −4.98126 | | −3.05747 |
| 4952 | OCULOCEREBRORENAL SYNDROME OF LOWE | OCRL | −3.56185 | | −3.44429 |
| 4957 | OUTER DENSE FIBER OF SPERM TAILS 2 | ODF2 | −1.98896 | −2.06861 | |
| 169611 | OLFACTOMEDIN-LIKE 2A | OLFML2A | −2.18173 | −2.26417 | |
| 10133 | OPTINEURIN | OPTN | −3.13476 | −2.18057 | |
| 138802 | OLFACTORY RECEPTOR, FAMILY 13, SUBFAMILY C, MEMBER 8 | OR13C8 | | −4.09071 | −2.51101 |
| 4991 | OLFACTORY RECEPTOR, FAMILY 1, SUBFAMILY D, MEMBER 2 | OR1D2 | −3.16865 | −3.64961 | −3.4325 |
| 347168 | OLFACTORY RECEPTOR, FAMILY 1, SUBFAMILY J, MEMBER 1 | OR1J1 | −2.01037 | −2.05408 | −3.5605 |
| 392392 | OLFACTORY RECEPTOR, FAMILY 1, SUBFAMILY K, MEMBER 1 | OR1K1 | −3.90467 | −2.16026 | |
| 144125 | OLFACTORY RECEPTOR, FAMILY 2, SUBFAMILY AG, MEMBER 1 | OR2AG1 | −1.90182 | −1.93355 | |
| 341152 | OLFACTORY RECEPTOR, FAMILY 2, SUBFAMILY AT, MEMBER 4 | OR2AT4 | −2.69945 | −2.16615 | |
| 81469 | OLFACTORY RECEPTOR, FAMILY 2, SUBFAMILY G, MEMBER 3 | OR2G3 | −1.96137 | | −2.16489 |
| 391194 | OLFACTORY RECEPTOR, FAMILY 2, SUBFAMILY M, MEMBER 2 | OR2M2 | | −2.41789 | −1.98612 |
| 26245 | OLFACTORY RECEPTOR, FAMILY 2, SUBFAMILY M, MEMBER 4 | OR2M4 | | −2.72931 | −2.1423 |
| 127069 | OLFACTORY RECEPTOR, FAMILY 2, SUBFAMILY T, MEMBER 10 | OR2T10 | −3.65767 | −2.24491 | |
| 284383 | OLFACTORY RECEPTOR, FAMILY 2, SUBFAMILY Z, MEMBER 1 | OR2Z1 | −3.07678 | −4.72263 | |
| 219429 | OLFACTORY RECEPTOR, FAMILY 4, SUBFAMILY C, MEMBER 11 | OR4C11 | −2.64811 | −2.09856 | |
| 79317 | OLFACTORY RECEPTOR, FAMILY 4, SUBFAMILY K, MEMBER 5 | OR4K5 | −2.60077 | −1.91433 | |
| 81300 | OLFACTORY RECEPTOR, FAMILY 4, SUBFAMILY P, MEMBER 4 | OR4P4 | −4.49028 | −2.64832 | −2.25624 |
| 256148 | OLFACTORY RECEPTOR, FAMILY 4, SUBFAMILY S, MEMBER 1 | OR4S1 | −2.26199 | −1.94054 | |
| 196335 | OLFACTORY RECEPTOR, FAMILY 56, SUBFAMILY B, MEMBER 4 | OR56B4 | −5.35355 | −1.91302 | |
| 403274 | OLFACTORY RECEPTOR, FAMILY 5, SUBFAMILY H, MEMBER 15 | OR5H15 | −6.03036 | | −2.24129 |
| 120065 | OLFACTORY RECEPTOR, FAMILY 5, SUBFAMILY P, MEMBER 2 | OR5P2 | −2.71333 | | −2.64519 |
| 390154 | OLFACTORY RECEPTOR, FAMILY 5, SUBFAMILY T, MEMBER 3 | OR5T3 | −1.89065 | | −2.19858 |
| 391114 | OLFACTORY RECEPTOR, FAMILY 6, SUBFAMILY K, MEMBER 3 | OR6K3 | | −2.47585 | −2.64133 |
| 10956 | AMPLIFIED IN OSTEOSARCOMA | OS9 | | −1.88867 | −2.8692 |
| 55074 | OXIDATION RESISTANCE 1 | OXR1 | −2.56001 | −3.04851 | |
| 54995 | 3-OXOACYL-ACP SYNTHASE, MITOCHONDRIAL | OXSM | | −2.55496 | −2.11533 |
| 125988 | QIL1 PROTEIN | P117 | −3.33321 | | −1.88138 |
| 23241 | PHOSPHOFURIN ACIDIC CLUSTER SORTING PROTEIN 2 | PACS2 | −2.36166 | | −2.96823 |
| 5050 | PLATELET-ACTIVATING FACTOR ACETYLHYDROLASE, ISOFORM IB, | PAFAH1B3 | −1.90165 | | −2.03995 |
| | GAMMA SUBUNIT 29 KDA | | | | |
| 23022 | PALLADIN, CYTOSKELETAL ASSOCIATED PROTEIN | PALLD | −1.87671 | | −2.14591 |
| 10914 | POLY(A) POLYMERASE ALPHA | PAPOLA | −2.08643 | −4.05228 | |
| 124222 | PROGESTIN AND ADIPOQ RECEPTOR FAMILY MEMBER IV | PAQR4 | −2.39582 | | −3.38779 |
| 142 | POLY (ADP-RIBOSE) POLYMERASE FAMILY, MEMBER 1 | PARP1 | | −1.92528 | −1.90532 |
| 143 | POLY (ADP-RIBOSE) POLYMERASE FAMILY, MEMBER 4 | PARP4 | | −2.40935 | −2.06206 |
| 27253 | PROTOCADHERIN 17 | PCDH17 | | −2.21436 | −3.0782 |
| 57526 | PROTOCADHERIN 19 | PCDH19 | −3.15028 | −2.86698 | |
| 56132 | PROTOCADHERIN BETA 3 | PCDHB3 | | −2.6344 | −2.62729 |
| 10336 | POLYCOMB GROUP RING FINGER 3 | PCGF3 | −3.03471 | | −1.89414 |
| 84333 | POLYCOMB GROUP RING FINGER 5 | PCGF5 | −2.773 | −1.96929 | −4.09651 |
| 55795 | HYPOTHETICAL PROTEIN FLJ11305 | PCID2 | | −1.98532 | −2.32044 |
| 5046 | PROPROTEIN CONVERTASE SUBTILISIN/KEXIN TYPE 6 | PCSK6 | −2.1397 | −3.10903 | |
| 58488 | PHOSPHATIDYLCHOLINE TRANSFER PROTEIN | PCTP | −6.35862 | −3.17278 | −1.87497 |
| 5161 | PYRUVATE DEHYDROGENASE (LIPOAMIDE) ALPHA 2 | PDHA2 | −2.027 | −3.19203 | |
| 5166 | PYRUVATE DEHYDROGENASE KINASE, ISOZYME 4 | PDK4 | −2.08325 | | −1.86973 |
| 9260 | PDZ AND LIM DOMAIN 7 (ENIGMA) | PDLIM7 | | −2.14491 | −1.88778 |
| 57546 | PYRUVATE DEHYDROGENASE PHOSPHATASE ISOENZYME 2 | PDP2 | −2.63359 | −2.24757 | |
| 3651 | INSULIN PROMOTER FACTOR 1, HOMEODOMAIN TRANSCRIPTION | PDX1 | | −2.14427 | −2.43953 |
| | FACTOR | | | | |
| 51248 | PDZ DOMAIN CONTAINING 11 | PDZD11 | −2.05074 | −2.52156 | |
| 5179 | PROENKEPHALIN | PENK | −1.99554 | −2.52421 | |
| 64065 | PERP, TP53 APOPTOSIS EFFECTOR | PERP | | −3.04715 | −1.86263 |
| 5210 | 6-PHOSPHOFRUCTO-2-KINASE/FRUCTOSE-2,6-BIPHOSPHATASE 4 | PFKFB4 | −2.80393 | | −2.73894 |
| 80055 | GPI DEACYLASE | PGAP1 | −2.47747 | −4.57794 | −2.70667 |
| 267004 | EXCISION REPAIR CROSS-COMPLEMENTING RODENT REPAIR | PGBD3 | −4.20479 | | −1.88145 |
| | DEFICIENCY, COMPLEMENTATION GROUP 6 | | | | |
| 57115 | PEPTIDOGLYCAN RECOGNITION PROTEIN 4 | PGLYRP4 | | −1.99135 | −1.85699 |
| 84680 | 1-AMINOCYCLOPROPANE-1-CARBOXYLATE SYNTHASE | PHACS | −3.19355 | −2.15665 | |
| 84295 | PHD FINGER PROTEIN 6 | PHF6 | −2.28931 | −2.38237 | |
| 22822 | PLECKSTRIN HOMOLOGY-LIKE DOMAIN, FAMILY A, MEMBER 1 | PHLDA1 | −2.63006 | | −2.53575 |
| 221476 | PEPTIDASE INHIBITOR 16 | PI16 | −2.17842 | | −2.47888 |
| 5277 | PHOSPHATIDYLINOSITOL GLYCAN, CLASS A (PAROXYSMAL | PIGA | −2.7267 | | −1.97114 |
| | NOCTURNAL HEMOGLOBINURIA) | | | | |
| 5289 | PHOSPHOINOSITIDE-3-KINASE, CLASS 3 | PIK3C3 | −3.02798 | −2.16509 | |
| 65018 | PTEN INDUCED PUTATIVE KINASE 1 | PINK1 | −2.65238 | −6.72753 | −4.89433 |
| 54984 | PIN2-INTERACTING PROTEIN 1 | PINX1 | −2.13509 | −2.25069 | |
| 8395 | PHOSPHATIDYLINOSITOL-4-PHOSPHATE 5-KINASE, TYPE I, BETA | PIP5K1B | −2.49714 | −3.11396 | −2.30031 |
| 23761 | PHOSPHATIDYLSERINE DECARBOXYLASE | PISD | −2.77933 | −3.93785 | |
| 5314 | POLYCYSTIC KIDNEY AND HEPATIC DISEASE 1 (AUTOSOMAL | PKHD1 | −1.95223 | −2.59701 | |
| | RECESSIVE) | | | | |
| 5569 | PROTEIN KINASE (CAMP-DEPENDENT, CATALYTIC) INHIBITOR ALPHA | PKIA | −3.57576 | −2.04651 | |
| 29941 | PROTEIN KINASE N3 | PKN3 | | −2.10742 | −2.15859 |
| 5318 | PLAKOPHILIN 2 | PKP2 | −2.89785 | −2.30137 | |
| 283748 | PHOSPHOLIPASE A2, GROUP IVD (CYTOSOLIC) | PLA2G4D | −1.96541 | | 2.51478 |
| 5322 | PHOSPHOLIPASE A2, GROUP V | PLA2G5 | −2.68114 | | −2.08402 |
| 5326 | PLEIOMORPHIC ADENOMA GENE-LIKE 2 | PLAGL2 | | −4.21387 | −2.5165 |
| 5327 | PLASMINOGEN ACTIVATOR, TISSUE | PLAT | | −3.25981 | −3.48747 |
| 5332 | PHOSPHOLIPASE C, BETA 4 | PLCB4 | −2.49701 | −2.10791 | |
| 257068 | PHOSPHATIDYLINOSITOL-SPECIFIC PHOSPHOLIPASE C, X DOMAIN | PLCXD2 | −3.6423 | −2.74439 | |
| | CONTAINING 2 | | | | |
| 54477 | PLECKSTRIN HOMOLOGY DOMAIN CONTAINING, FAMILY A MEMBER 5 | PLEKHA5 | −2.2379 | −2.30195 | |
| 58473 | PLECKSTRIN HOMOLOGY DOMAIN CONTAINING, FAMILY B (EVECTINS) | PLEKHB1 | | −2.58955 | −2.51903 |
| | MEMBER 1 | | | | |
| 11284 | POLYNUCLEOTIDE KINASE 3′-PHOSPHATASE | PNKP | −1.97244 | −2.18221 | −2.14383 |
| 79883 | HYPOTHETICAL PROTEIN FLJ23447 | PODNL1 | −4.69966 | −2.85234 | |
| 79001 | VITAMIN K EPOXIDE REDUCTASE COMPLEX, SUBUNIT 1 | POL3S | | −2.15713 | −2.25578 |
| 23649 | POLYMERASE (DNA DIRECTED), ALPHA 2 (70 KD SUBUNIT) | POLA2 | −2.98897 | −2.75611 | |
| 54107 | POLYMERASE (DNA DIRECTED), EPSILON 3 (P17 SUBUNIT) | POLE3 | −3.28031 | −2.07165 | −2.89831 |
| 94026 | POM121 MEMBRANE GLYCOPROTEIN-LIKE 2 (RAT) | POM121L2 | −4.64585 | | −2.05884 |
| 29954 | PROTEIN-O-MANNOSYLTRANSFERASE 2 | POMT2 | −2.84072 | | −1.962 |
| 5446 | PARAOXONASE 3 | PON3 | −4.70654 | −2.34705 | |
| 10940 | PROCESSING OF PRECURSOR 1, RIBONUCLEASE P/MRP SUBUNIT | POP1 | −4.49314 | −2.46298 | −1.97797 |
| | (S. CEREVISIAE) | | | | |
| 25833 | POU DOMAIN, CLASS 2, TRANSCRIPTION FACTOR 3 | POU2F3 | −3.34571 | −2.41621 | −2.29749 |
| 8612 | PHOSPHATIDIC ACID PHOSPHATASE TYPE 2C | PPAP2C | −2.95992 | −2.07329 | |
| 84814 | PHOSPHATIDIC ACID PHOSPHATASE TYPE 2 DOMAIN CONTAINING 3 | PPAPDC3 | −1.95667 | | −1.96121 |
| 8499 | PROTEIN TYROSINE PHOSPHATASE, RECEPTOR TYPE, F POLYPEPTIDE | PPFIA2 | | −2.04642 | −2.65517 |
| | (PTPRF), INTERACTING PROTEIN (LIPRIN), ALPHA 2 | | | | |
| 9360 | PEPTIDYLPROLYL ISOMERASE G (CYCLOPHILIN G) | PPIG | −3.51287 | | −2.43114 |
| 5498 | PROTOPORPHYRINOGEN OXIDASE | PPOX | −1.91497 | −2.02656 | |
| 4659 | PROTEIN PHOSPHATASE 1, REGULATORY (INHIBITOR) SUBUNIT 12A | PPP1R12A | −2.86581 | | −2.07108 |
| 79660 | PROTEIN PHOSPHATASE 1, REGULATORY (INHIBITOR) SUBUNIT 3B | PPP1R3B | −3.05123 | −1.9493 | |
| 5515 | PROTEIN PHOSPHATASE 2 (FORMERLY 2A), CATALYTIC SUBUNIT, | PPP2CB | −5.1951 | −3.82763 | |
| | ALPHA ISOFORM | | | | |
| 55012 | CHROMOSOME 14 OPEN READING FRAME 10 | PPP2R3C | −1.90047 | −2.87177 | −1.99879 |
| 5525 | PROTEIN PHOSPHATASE 2, REGULATORY SUBUNIT B (B56), ALPHA | PPP2R5A | −1.85237 | −1.88221 | |
| | ISOFORM | | | | |
| 23082 | PEROXISOME PROLIFERATIVE ACTIVATED RECEPTOR, GAMMA, | PPRC1 | −3.33778 | | −2.53909 |
| | COACTIVATOR-RELATED 1 | | | | |
| 65121 | PRAME FAMILY MEMBER 1 | PRAMEF12 | −1.95611 | −2.12128 | |
| 9055 | PROTEIN REGULATOR OF CYTOKINESIS 1 | PRC1 | −4.69961 | −2.34331 | −2.33972 |
| 5551 | PERFORIN 1 (PORE FORMING PROTEIN) | PRF1 | −3.11112 | −4.12973 | −3.64376 |
| 5562 | PROTEIN KINASE, AMP-ACTIVATED, ALPHA 1 CATALYTIC SUBUNIT | PRKAA1 | | −2.42841 | −3.18606 |
| 5568 | PROTEIN KINASE, CAMP-DEPENDENT, CATALYTIC, GAMMA | PRKACG | | −2.27403 | −2.2523 |
| 56341 | PROTEIN ARGININE METHYLTRANSFERASE 8 | PRMT8 | −2.53607 | | −2.38768 |
| 5626 | PROPHET OF PIT1, PAIRED-LIKE HOMEODOMAIN TRANSCRIPTION | PROP1 | | −3.17865 | −1.91607 |
| | FACTOR | | | | |
| 51334 | MESENCHYMAL STEM CELL PROTEIN DSC54 | PRR16 | −1.89517 | −2.22191 | |
| 10279 | PROTEASE, SERINE, 16 (THYMUS) | PRSS16 | −2.13449 | −2.63428 | −3.12227 |
| 400668 | PROTEASE, SERINE-LIKE 1 | PRSSL1 | | −1.87807 | −2.17207 |
| 57716 | PERIAXIN | PRX | | −3.7748 | −2.19862 |
| 9595 | PLECKSTRIN HOMOLOGY, SEC7 AND COILED-COIL DOMAINS, BINDING | PSCDBP | | −2.27685 | −2.20532 |
| | PROTEIN | | | | |
| 5681 | PROTEIN SERINE KINASE H1 | PSKH1 | −2.63033 | | −2.63583 |
| 139411 | PATCHED DOMAIN CONTAINING 1 | PTCHD1 | −2.0655 | −2.79843 | |
| 81490 | PHOSPHATIDYLSERINE SYNTHASE 2 | PTDSS2 | | −2.19403 | −1.9922 |
| 11099 | PROTEIN TYROSINE PHOSPHATASE, NON-RECEPTOR TYPE 21 | PTPN21 | −2.49387 | −2.04587 | |
| 5787 | PROTEIN TYROSINE PHOSPHATASE, RECEPTOR TYPE, B | PTPRB | −2.9585 | −2.07646 | |
| 5794 | PROTEIN TYROSINE PHOSPHATASE, RECEPTOR TYPE, H | PTPRH | −2.91132 | −2.41963 | |
| 5801 | PROTEIN TYROSINE PHOSPHATASE, RECEPTOR TYPE, R | PTPRR | | −2.05897 | −2.10197 |
| 5814 | PURINE-RICH ELEMENT BINDING PROTEIN B | PURB | −3.03529 | −2.0988 | |
| 79912 | HYPOTHETICAL PROTEIN FLJ22028 | PYROXD1 | −2.08054 | −3.59087 | −3.50611 |
| 5697 | PEPTIDE YY | PYY | −3.01242 | −1.89304 | |
| 9727 | RAB11 FAMILY INTERACTING PROTEIN 3 (CLASS II) | RAB11FIP3 | −1.93666 | −2.06647 | |
| 401409 | GTP-BINDING PROTEIN RAB19B | RAB19 | −4.16718 | −2.50667 | |
| 11021 | RAB35, MEMBER RAS ONCOGENE FAMILY | RAB35 | −3.72915 | −2.10077 | |
| 116442 | RAB39B, MEMBER RAS ONCOGENE FAMILY | RAB39B | −3.40849 | −2.77826 | −2.55766 |
| 5867 | RAB4A, MEMBER RAS ONCOGENE FAMILY | RAB4A | | −2.23824 | −2.38798 |
| 53916 | RAB4B, MEMBER RAS ONCOGENE FAMILY | RAB4B | −2.62903 | −2.16647 | |
| 5885 | RAD21 HOMOLOG (S. POMBE) | RAD21 | | −3.13061 | −2.07017 |
| 23132 | RAD54-LIKE 2 (S. CEREVISIAE) | RAD54L2 | | −2.48403 | −3.18984 |
| 5883 | RAD9 HOMOLOG A (S. POMBE) | RAD9A | −1.98876 | −2.71587 | −2.70529 |
| 22913 | RNA BINDING PROTEIN, AUTOANTIGENIC (HNRNP-ASSOCIATED WITH | RALY | | −2.31833 | −2.07084 |
| | LETHAL YELLOW HOMOLOG (MOUSE)) | | | | |
| 26953 | RAN BINDING PROTEIN 6 | RANBP6 | −2.83259 | | −1.8949 |
| 5906 | RAP1A, MEMBER OF RAS ONCOGENE FAMILY | RAP1A | −2.15637 | −2.59656 | |
| 5920 | RETINOIC ACID RECEPTOR RESPONDER (TAZAROTENE INDUCED) 3 | RARRES3 | −3.59647 | −2.88117 | −3.01349 |
| 25780 | RAS GUANYL RELEASING PROTEIN 3 (CALCIUM AND DAG-REGULATED) | RASGRP3 | | −2.68079 | −2.15504 |
| 64080 | RIBOKINASE | RBKS | −2.70105 | −3.24448 | |
| 54033 | RNA BINDING MOTIF PROTEIN 11 | RBM11 | | −3.16829 | −1.94764 |
| 166863 | HYPOTHETICAL PROTEIN MGC27016 | RBM46 | −2.66251 | | −2.8393 |
| 23543 | RNA BINDING MOTIF PROTEIN 9 | RBM9 | −4.51469 | −2.6588 | |
| 83758 | RETINOL BINDING PROTEIN 5, CELLULAR | RBP5 | −2.07092 | −2.56384 | −2.33793 |
| 11317 | RECOMBINING BINDING PROTEIN SUPPRESSOR OF HAIRLESS | RBPJL | −3.4381 | −2.8404 | |
| | (DROSOPHILA)-LIKE | | | | |
| 348093 | RNA BINDING PROTEIN WITH MULTIPLE SPLICING 2 | RBPMS2 | −4.50172 | −3.38917 | −2.0127 |
| 5957 | RECOVERIN | RCVRN | | −2.95204 | −3.18587 |
| 7936 | RD RNA BINDING PROTEIN | RDBP | −2.08726 | −2.16704 | |
| 5962 | RADIXIN | RDX | −2.37925 | −2.62954 | |
| 51308 | RECEPTOR ACCESSORY PROTEIN 2 | REEP2 | −2.85997 | −1.96118 | |
| 56729 | RESISTIN | RETN | −1.93284 | −1.90428 | −2.73758 |
| 55312 | RIBOFLAVIN KINASE | RFK | | −1.90106 | −2.43082 |
| 442247 | SIMILAR TO RET FINGER PROTEIN-LIKE 1 | RFPL4B | −3.39753 | −2.28722 | |
| 93587 | RNA (GUANINE-9-) METHYLTRANSFERASE DOMAIN CONTAINING 2 | RG9MTD2 | −3.08085 | −2.39402 | |
| 5996 | REGULATOR OF G-PROTEIN SIGNALLING 1 | RGS1 | −2.6945 | −4.44163 | |
| 26166 | REGULATOR OF G-PROTEIN SIGNALLING 22 | RGS22 | −1.99317 | −2.56763 | |
| 121268 | RAS HOMOLOG ENRICHED IN BRAIN LIKE 1 | RHEBL1 | −3.3124 | −2.59878 | |
| 54509 | RAS HOMOLOG GENE FAMILY, MEMBER F (IN FILOPODIA) | RHOF | −1.99428 | | −2.60677 |
| 399 | RAS HOMOLOG GENE FAMILY, MEMBER H | RHOH | −2.87756 | −2.60693 | |
| 25778 | RECEPTOR INTERACTING PROTEIN KINASE 5 | RIPK5 | −2.16914 | | −3.07716 |
| 6039 | RIBONUCLEASE, RNASE A FAMILY, K6 | RNASE6 | −3.83481 | −3.37285 | |
| 140432 | RING FINGER PROTEIN 113B | RNF113B | | −2.17001 | −2.80417 |
| 79845 | RING FINGER PROTEIN 122 | RNF122 | −2.16016 | −3.10137 | |
| 54546 | RING FINGER PROTEIN 186 | RNF186 | −1.95557 | | −2.34727 |
| 6050 | RIBONUCLEASE/ANGIOGENIN INHIBITOR 1 | RNH1 | | −1.85151 | −2.2123 |
| 10921 | RNA BINDING PROTEIN S1, SERINE-RICH DOMAIN | RNPS1 | −2.23163 | −2.8619 | |
| 10556 | RIBONUCLEASE P/MRP 30 KDA SUBUNIT | RPP30 | −2.7066 | −3.00534 | −2.13315 |
| 56261 | HYPOTHETICAL PROTEIN KIAA1434 | RPS18P1 | −2.85418 | −2.69983 | |
| 91582 | RIBOSOMAL PROTEIN S19 BINDING PROTEIN 1 | RPS19BP1 | −2.21276 | −2.78638 | |
| 8986 | RIBOSOMAL PROTEIN S6 KINASE, 90 KDA, POLYPEPTIDE 4 | RPS6KA4 | −1.93966 | −2.88556 | |
| 9136 | RNA, U3 SMALL NUCLEOLAR INTERACTING PROTEIN 2 | RRP9 | −2.34514 | | −1.86564 |
| 84870 | R-SPONDIN 3 HOMOLOG (XENOPUS LAEVIS) | RSPO3 | −2.13463 | −2.68373 | |
| 146760 | RETICULON 4 RECEPTOR-LIKE 1 | RTN4RL1 | | −2.07964 | −2.34172 |
| 146923 | RUN DOMAIN CONTAINING 1 | RUNDC1 | −2.18962 | −2.04771 | |
| 154661 | RAP2-BINDING PROTEIN 9 | RUNDC3B | | −2.9338 | −3.37057 |
| 862 | RUNT-RELATED TRANSCRIPTION FACTOR 1; TRANSLOCATED TO, 1 | RUNX1T1 | −2.12615 | −2.27231 | |
| | (CYCLIN D-RELATED) | | | | |
| 23429 | RING1 AND YY1 BINDING PROTEIN | RYBP | | −2.02569 | −2.10047 |
| 645922 | SIMILAR TO S100 CALCIUM BINDING PROTEIN A7-LIKE 1 | S100A7L2 | −3.42865 | −2.28363 | −2.64235 |
| 6285 | S100 CALCIUM BINDING PROTEIN, BETA (NEURAL) | S100B | −2.2964 | | −3.721 |
| 113174 | SERUM AMYLOID A-LIKE 1 | SAAL1 | −3.11984 | | −3.5017 |
| 27164 | SAL-LIKE 3 (DROSOPHILA) | SALL3 | | −2.34564 | −2.04659 |
| 344658 | STERILE ALPHA MOTIF DOMAIN CONTAINING 7 | SAMD7 | | −2.51076 | −2.13509 |
| 55291 | CHROMOSOME 11 OPEN READING FRAME 23 | SAPS3 | | −2.90252 | −1.8513 |
| 163786 | SPINDLE ASSEMBLY 6 HOMOLOG (C. ELEGANS) | SASS6 | | −1.97416 | −2.54308 |
| 23314 | SATB FAMILY MEMBER 2 | SATB2 | | −2.85258 | −2.07986 |
| 23256 | SEC1 FAMILY DOMAIN CONTAINING 1 | SCFD1 | −2.41869 | −3.0156 | |
| 7857 | SECRETOGRANIN II (CHROMOGRANIN C) | SCG2 | −2.43482 | | −2.45974 |
| 6332 | SODIUM CHANNEL, VOLTAGE-GATED, TYPE VII, ALPHA | SCN7A | −4.12895 | −2.66506 | |
| 51246 | SCOTIN | SCOTIN | −4.07991 | −2.10949 | |
| 6343 | SECRETIN | SCT | −4.44056 | −2.51525 | |
| 80274 | SIGNAL PEPTIDE, CUB DOMAIN, EGF-LIKE 1 | SCUBE1 | | −2.61249 | −2.47153 |
| 9255 | SMALL INDUCIBLE CYTOKINE SUBFAMILY E, MEMBER 1 (ENDOTHELIAL | SCYE1 | −2.36951 | −2.70046 | |
| | MONOCYTE-ACTIVATING) | | | | |
| 9672 | SYNDECAN 3 (N-SYNDECAN) | SDC3 | −2.91628 | | −2.81579 |
| 29927 | SEC61 ALPHA 1 SUBUNIT (S. CEREVISIAE) | SEC61A1 | −1.88201 | −3.10851 | |
| 55176 | SEC61 ALPHA 2 SUBUNIT (S. CEREVISIAE) | SEC61A2 | | −1.90727 | −2.15394 |
| 10952 | SEC61 BETA SUBUNIT | SEC61B | | −1.90117 | −2.31682 |
| 6404 | SELECTIN P LIGAND | SELPLG | −2.09831 | −3.20512 | |
| 348303 | SELENOPROTEIN V | SELV | | −3.17995 | −2.28173 |
| 9037 | SEMA DOMAIN, SEVEN THROMBOSPONDIN REPEATS (TYPE 1 AND | SEMA5A | | −2.55799 | −2.73749 |
| | TYPE 1-LIKE), TRANSMEMBRANE DOMAIN (TM) AND SHORT | | | | |
| | CYTOPLASMIC DOMAIN, (SEMAPHORIN) 5A | | | | |
| 10500 | SEMA DOMAIN, TRANSMEMBRANE DOMAIN (TM), AND CYTOPLASMIC | SEMA6C | −3.43802 | −1.91085 | |
| | DOMAIN, (SEMAPHORIN) 6C | | | | |
| 26054 | SUMO1/SENTRIN SPECIFIC PEPTIDASE 6 | SENP6 | −2.12559 | −4.92084 | |
| 1992 | SERPIN PEPTIDASE INHIBITOR, CLADE B (OVALBUMIN), MEMBER 1 | SERPINB1 | −3.26772 | −2.86627 | −2.8311 |
| 462 | SERPIN PEPTIDASE INHIBITOR, CLADE C (ANTITHROMBIN), MEMBER 1 | SERPINC1 | | −2.62448 | −1.95094 |
| 143686 | SESTRIN 3 | SESN3 | −2.41865 | −2.54664 | |
| 387893 | SET DOMAIN CONTAINING (LYSINE METHYLTRANSFERASE) 8 | SETD8 | −2.21435 | −2.14105 | |
| 9295 | SPLICING FACTOR, ARGININE/SERINE-RICH 11 | SFRS11 | −3.46224 | −2.01065 | |
| 25957 | CHROMOSOME 6 OPEN READING FRAME 111 | SFRS18 | −2.8846 | −2.66063 | |
| 6457 | SH3-DOMAIN GRB2-LIKE 3 | SH3GL3 | −2.09546 | | −1.90088 |
| 22941 | SH3 AND MULTIPLE ANKYRIN REPEAT DOMAINS 2 | SHANK2 | −1.85617 | −2.35166 | |
| 6462 | SEX HORMONE-BINDING GLOBULIN | SHBG | | −2.12767 | −3.0562 |
| 134549 | APICAL PROTEIN 2 | SHROOM1 | −2.68342 | −2.30012 | |
| 25942 | SIN3 HOMOLOG A, TRANSCRIPTION REGULATOR (YEAST) | SIN3A | −2.50484 | | −1.91435 |
| 23094 | SIGNAL-INDUCED PROLIFERATION-ASSOCIATED 1 LIKE 3 | SIPA1L3 | | −2.00188 | −1.98104 |
| 6498 | SKI-LIKE | SKIL | −2.52428 | −2.56457 | −2.61359 |
| 84174 | SRC-LIKE-ADAPTOR 2 | SLA2 | | −3.20957 | −2.37006 |
| 4891 | SOLUTE CARRIER FAMILY 11 (PROTON-COUPLED DIVALENT METAL ION | SLC11A2 | −2.54813 | −3.59077 | |
| | TRANSPORTERS), MEMBER 2 | | | | |
| 6560 | SOLUTE CARRIER FAMILY 12 (POTASSIUM/CHLORIDE TRANSPORTERS), | SLC12A4 | −1.91482 | −2.6526 | −3.08265 |
| | MEMBER 4 | | | | |
| 117247 | HYPOTHETICAL PROTEIN PRO0813 | SLC16A10 | −2.62914 | −2.48375 | |
| 55356 | SOLUTE CARRIER FAMILY 22 (ORGANIC CATION TRANSPORTER), | SLC22A15 | −2.86114 | −2.176 | |
| | MEMBER 15 | | | | |
| 8604 | SOLUTE CARRIER FAMILY 25 (MITOCHONDRIAL CARRIER, ARALAR), | SLC25A12 | −3.02631 | | −2.05383 |
| | MEMBER 12 | | | | |
| 115286 | SOLUTE CARRIER FAMILY 25, MEMBER 26 | SLC25A26 | −4.64485 | −4.72509 | |
| 291 | SOLUTE CARRIER FAMILY 25 (MITOCHONDRIAL CARRIER; ADENINE | SLC25A4 | −2.38678 | −2.19747 | |
| | NUCLEOTIDE TRANSLOCATOR), MEMBER 4 | | | | |
| 11001 | FATTY-ACID-COENZYME A LIGASE, VERY LONG-CHAIN 1 | SLC27A2 | −2.29279 | | −2.10208 |
| 11000 | SOLUTE CARRIER FAMILY 27 (FATTY ACID TRANSPORTER), MEMBER 3 | SLC27A3 | | −1.86814 | −2.04285 |
| 64078 | SOLUTE CARRIER FAMILY 28 (SODIUM-COUPLED NUCLEOSIDE | SLC28A3 | −2.72258 | | −2.00847 |
| | TRANSPORTER), MEMBER 3 | | | | |
| 54733 | SOLUTE CARRIER FAMILY 35, MEMBER F2 | SLC35F2 | −3.54646 | −2.37764 | |
| 23446 | SOLUTE CARRIER FAMILY 44, MEMBER 1 | SLC44A1 | −2.74664 | −2.60586 | |
| 9152 | SOLUTE CARRIER FAMILY 6 (NEUROTRANSMITTER TRANSPORTER, | SLC6A5 | −3.13789 | | −2.174 |
| | GLYCINE), MEMBER 5 | | | | |
| 6550 | SOLUTE CARRIER FAMILY 9 (SODIUM/HYDROGEN EXCHANGER), | SLC9A3 | −2.10203 | −2.83787 | |
| | MEMBER 3 | | | | |
| 9351 | SOLUTE CARRIER FAMILY 9 (SODIUM/HYDROGEN EXCHANGER), | SLC9A3R2 | | −1.99031 | −2.77691 |
| | MEMBER 3 REGULATOR 2 | | | | |
| 4088 | SMAD, MOTHERS AGAINST DPP HOMOLOG 3 (DROSOPHILA) | SMAD3 | −2.1217 | | −1.97539 |
| 60682 | STROMAL MEMBRANE-ASSOCIATED PROTEIN 1 | SMAP1 | −4.06345 | | −2.32987 |
| 6603 | SWI/SNF RELATED, MATRIX ASSOCIATED, ACTIN DEPENDENT | SMARCD2 | | −5.85968 | −2.70953 |
| | REGULATOR OF CHROMATIN, SUBFAMILY D, MEMBER 2 | | | | |
| 79677 | SMC6 STRUCTURAL MAINTENANCE OF CHROMOSOMES 6-LIKE 1 | SMC6 | | −2.24287 | −3.68918 |
| | (YEAST) | | | | |
| 6609 | SPHINGOMYELIN PHOSPHODIESTERASE 1, ACID LYSOSOMAL (ACID | SMPD1 | −4.41417 | −3.32313 | |
| | SPHINGOMYELINASE) | | | | |
| 6525 | SMOOTHELIN | SMTN | −1.97754 | −4.5857 | |
| 9751 | SYNTAPHILIN | SNPH | −2.19133 | −3.77141 | |
| 6629 | SMALL NUCLEAR RIBONUCLEOPROTEIN POLYPEPTIDE B” | SNRPB2 | −2.3241 | −2.37559 | |
| 54212 | SYNTROPHIN, GAMMA 1 | SNTG1 | −2.50921 | −2.49363 | |
| 29916 | SORTING NEXIN 11 | SNX11 | −2.01535 | −1.93444 | |
| 23161 | SORTING NEXIN 13 | SNX13 | −2.0599 | −2.51066 | |
| 6652 | SORBITOL DEHYDROGENASE | SORD | −3.55463 | −2.14119 | −1.97628 |
| 6655 | SON OF SEVENLESS HOMOLOG 2 (DROSOPHILA) | SOS2 | −2.82991 | −2.64977 | |
| 9580 | SRY (SEX DETERMINING REGION Y)-BOX 13 | SOX13 | −3.5731 | −3.49442 | |
| 6667 | SP1 TRANSCRIPTION FACTOR | SP1 | −2.90651 | −3.43006 | |
| 10615 | SPERM ASSOCIATED ANTIGEN 5 | SPAG5 | −4.34344 | −2.63317 | |
| 80309 | SPHK1 (SPHINGOSINE KINASE TYPE 1) INTERACTING PROTEIN | SPHKAP | −2.35706 | −3.18283 | |
| 643394 | SIMILAR TO ESOPHAGUS CANCER-RELATED GENE 2 PROTEIN | SPINK9 | −3.44822 | −3.26333 | |
| | PRECURSOR (ECRG-2) | | | | |
| 83985 | SPINSTER | SPNS1 | −2.93729 | −2.13496 | −3.33721 |
| 201305 | HYPOTHETICAL PROTEIN MGC29671 | SPNS3 | −2.26795 | −3.47607 | |
| 6695 | SPARC/OSTEONECTIN, CWCV AND KAZAL-LIKE DOMAINS | SPOCK1 | | −5.11991 | −3.69976 |
| | PROTEOGLYCAN (TESTICAN) 1 | | | | |
| 10418 | SPONDIN 1, EXTRACELLULAR MATRIX PROTEIN | SPON1 | −2.5145 | −3.43213 | −2.98118 |
| 6720 | STEROL REGULATORY ELEMENT BINDING TRANSCRIPTION FACTOR 1 | SREBF1 | −2.05248 | −1.98817 | |
| 51188 | SYNOVIAL SARCOMA TRANSLOCATION GENE ON CHROMOSOME 18- | SS18L2 | −2.55102 | −4.97016 | |
| | LIKE 2 | | | | |
| 23648 | SINGLE STRANDED DNA BINDING PROTEIN 3 | SSBP3 | −1.90861 | −3.22848 | |
| 23145 | SCO-SPONDIN HOMOLOG (BOS TAURUS) | SSPO | −3.57048 | −2.81785 | −4.46215 |
| 6745 | SIGNAL SEQUENCE RECEPTOR, ALPHA (TRANSLOCON-ASSOCIATED | SSR1 | −2.8973 | −3.21686 | −3.23373 |
| | PROTEIN ALPHA) | | | | |
| 9705 | SUPPRESSION OF TUMORIGENICITY 18 (BREAST CARCINOMA) (ZINC | ST18 | −3.4941 | | −3.10471 |
| | FINGER PROTEIN) | | | | |
| 29906 | ST8 ALPHA-N-ACETYL-NEURAMINIDE ALPHA-2,8-SIALYLTRANSFERASE 5 | ST8SIA5 | −3.72604 | | −2.45803 |
| 27067 | STAUFEN, RNA BINDING PROTEIN, HOMOLOG 2 (DROSOPHILA) | STAU2 | −1.94355 | −4.02935 | |
| 8576 | SERINE/THREONINE KINASE 16 | STK16 | −1.97744 | | −2.08507 |
| 29888 | STRIATIN, CALMODULIN BINDING PROTEIN 4 | STRN4 | −4.29577 | −3.06848 | |
| 29091 | SYNTAXIN BINDING PROTEIN 6 (AMISYN) | STXBP6 | | −2.09798 | −2.18455 |
| 51684 | SUPPRESSOR OF FUSED HOMOLOG (DROSOPHILA) | SUFU | −2.03606 | −3.28222 | −2.11306 |
| 285362 | SULFATASE MODIFYING FACTOR 1 | SUMF1 | −3.73367 | −2.48341 | |
| 6836 | SURFEIT 4 | SURF4 | −1.95495 | −5.84907 | |
| 23546 | SYNAPTOGYRIN 4 | SYNGR4 | −2.93405 | | −1.89975 |
| 171024 | SYNAPTOPODIN 2 | SYNPO2 | −3.58817 | | −3.00192 |
| 23208 | SYNAPTOTAGMIN XI | SYT11 | −3.85581 | −4.17378 | |
| 83849 | SYNAPTOTAGMIN XV | SYT15 | −3.73035 | −3.0071 | −2.76884 |
| 83851 | SYNAPTOTAGMIN XVI | SYT16 | | −2.98035 | −1.87882 |
| 90019 | SYNAPTOTAGMIN VIII | SYT8 | −3.26196 | −1.94023 | |
| 134864 | TRACE AMINE ASSOCIATED RECEPTOR 1 | TAAR1 | | −2.4933 | −2.37326 |
| 9287 | TRACE AMINE ASSOCIATED RECEPTOR 2 | TAAR2 | −2.07314 | −2.94412 | −2.55597 |
| 117143 | TRANSCRIPTIONAL ADAPTOR 1 (HFI1 HOMOLOG, YEAST)-LIKE | TADA1L | −3.58325 | −4.24081 | |
| 10474 | TRANSCRIPTIONAL ADAPTOR 3 (NGG1 HOMOLOG, YEAST)-LIKE | TADA3L | −3.69376 | −2.67301 | −2.67191 |
| 6883 | TAF12 RNA POLYMERASE II, TATA BOX BINDING PROTEIN (TBP)- | TAF12 | −3.506 | −3.03956 | −3.59767 |
| | ASSOCIATED FACTOR, 20 KDA | | | | |
| 8148 | TAF15 RNA POLYMERASE II, TATA BOX BINDING PROTEIN (TBP)- | TAF15 | −5.00259 | −2.99249 | |
| | ASSOCIATED FACTOR, 68 KDA | | | | |
| 389932 | ALDO-KETO REDUCTASE, TRUNCATED | TAKR | −3.01732 | −3.14658 | |
| 6888 | TRANSALDOLASE 1 | TALDO1 | −2.93536 | −1.951 | |
| 84807 | T-CELL ACTIVATION NFKB-LIKE PROTEIN | TA- | | −2.0215 | −2.34425 |
| | | NFKBH | | | |
| 374403 | TBC1 DOMAIN FAMILY, MEMBER 10C | TBC1D10C | −3.33335 | | −2.33856 |
| 23102 | TBC1 DOMAIN FAMILY, MEMBER 2B | TBC1D2B | −2.38151 | −3.63115 | −2.23145 |
| 79718 | TRANSDUCIN (BETA)-LIKE 1X-LINKED RECEPTOR 1 | TBL1XR1 | | −1.98849 | −2.93411 |
| 6926 | T-BOX 3 (ULNAR MAMMARY SYNDROME) | TBX3 | −2.84324 | −2.55287 | −2.1923 |
| 54103 | HYPOTHETICAL PROTEIN LOC54103 | TCAG7.1314 | | −1.97256 | −3.65763 |
| 56849 | TRANSCRIPTION ELONGATION FACTOR A (SII)-LIKE 7 | TCEAL7 | −4.02826 | −2.17249 | |
| 6921 | TRANSCRIPTION ELONGATION FACTOR B (SIII), POLYPEPTIDE 1 (15 KDA, | TCEB1 | −3.56259 | −2.87088 | −2.80807 |
| | ELONGIN C) | | | | |
| 9623 | T-CELL LEUKEMIA/LYMPHOMA 1B | TCL1B | −4.70516 | −2.58147 | −2.2034 |
| 7003 | TEA DOMAIN FAMILY MEMBER 1 (SV40 TRANSCRIPTIONAL ENHANCER | TEAD1 | −3.55384 | −2.12333 | |
| | FACTOR) | | | | |
| 7006 | TEC PROTEIN TYROSINE KINASE | TEC | −1.85863 | | −1.90836 |
| 23371 | TENSIN LIKE C1 DOMAIN CONTAINING PHOSPHATASE (TENSIN 2) | TENC1 | −3.23649 | −2.03696 | |
| 7011 | TELOMERASE-ASSOCIATED PROTEIN 1 | TEP1 | −2.33241 | −5.04989 | |
| 7022 | TRANSCRIPTION FACTOR AP-2 GAMMA (ACTIVATING ENHANCER | TFAP2C | | −2.50319 | −2.92926 |
| | BINDING PROTEIN 2 GAMMA) | | | | |
| 29842 | TRANSCRIPTION FACTOR CP2-LIKE 1 | TFCP2L1 | −2.75048 | −2.80252 | |
| 29844 | TCF3 (E2A) FUSION PARTNER (IN CHILDHOOD LEUKEMIA) | TFPT | −3.11717 | −3.06358 | −3.10235 |
| 51497 | TH1-LIKE (DROSOPHILA) | TH1L | −2.06756 | −2.99752 | |
| 353376 | TOLL-LIKE RECEPTOR ADAPTOR MOLECULE 2 | TICAM2 | −2.54594 | −1.92781 | |
| 7082 | TIGHT JUNCTION PROTEIN 1 (ZONA OCCLUDENS 1) | TJP1 | −1.99226 | | −3.30745 |
| 7083 | THYMIDINE KINASE 1, SOLUBLE | TK1 | −2.50739 | −2.82968 | |
| 9874 | TOUSLED-LIKE KINASE 1 | TLK1 | | −2.30245 | −2.34146 |
| 7092 | TOLLOID-LIKE 1 | TLL1 | −2.42975 | −2.34648 | |
| 51284 | TOLL-LIKE RECEPTOR 7 | TLR7 | −3.20969 | −2.41105 | |
| 9032 | TRANSMEMBRANE 4 L SIX FAMILY MEMBER 5 | TM4SF5 | | −2.0935 | −2.68228 |
| 53346 | TRANSMEMBRANE 6 SUPERFAMILY MEMBER 1 | TM6SF1 | −4.00854 | −2.48158 | |
| 51643 | TRANSMEMBRANE BAX INHIBITOR MOTIF CONTAINING 4 | TMBIM4 | −3.44325 | −1.93345 | |
| 79905 | TRANSMEMBRANE CHANNEL-LIKE 7 | TMC7 | −2.27616 | −1.85573 | −1.88393 |
| 55002 | TRANSMEMBRANE AND COILED-COIL DOMAINS 3 | TMCO3 | −2.37384 | −1.86547 | −2.07757 |
| 8834 | TRANSMEMBRANE PROTEIN 11 | TMEM11 | −4.15914 | −4.43887 | |
| 144404 | HYPOTHETICAL LOC144404 | TMEM120B | −5.92348 | −2.20834 | −2.36457 |
| 85014 | HYPOTHETICAL PROTEIN MGC14141 | TMEM141 | −3.93132 | −2.00305 | |
| 51522 | TRANSMEMBRANE PROTEIN 14C | TMEM14C | −3.83709 | −3.00587 | |
| 201799 | HYPOTHETICAL PROTEIN FLJ32028 | TMEM154 | −2.9453 | −4.0793 | −3.83264 |
| 55858 | TPA REGULATED LOCUS | TMEM165 | −3.00582 | −2.11807 | |
| 84286 | HYPOTHETICAL PROTEIN MGC4618 | TMEM175 | | −2.30215 | −1.95875 |
| 84548 | FAMILY WITH SEQUENCE SIMILARITY 11, MEMBER A | TMEM185A | −2.878 | −1.92037 | |
| 387521 | UBIQUITIN-CONJUGATING ENZYME VARIANT KUA | TMEM189 | −2.38212 | −3.47634 | |
| 55161 | TRANSMEMBRANE PROTEIN 33 | TMEM33 | | −1.85009 | −3.18274 |
| 131616 | TRANSMEMBRANE PROTEIN 42 | TMEM42 | −4.81996 | −2.71992 | |
| 51249 | TRANSMEMBRANE PROTEIN 69 | TMEM69 | −1.86253 | −2.34459 | |
| 144110 | TRANSMEMBRANE PROTEIN 86A | TMEM86A | −3.98965 | −2.15319 | |
| 641649 | TRANSMEMBRANE PROTEIN 91 | TMEM91 | −2.39084 | | −3.8996 |
| 126259 | HYPOTHETICAL PROTEIN MGC23244 | TMIGD2 | −2.19155 | −3.54299 | |
| 29766 | TROPOMODULIN 3 (UBIQUITOUS) | TMOD3 | −2.90096 | −3.05522 | |
| 344805 | TRANSMEMBRANE PROTEASE, SERINE 7 | TMPRSS7 | −2.22643 | −2.42787 | |
| 160335 | TRANSMEMBRANE AND TETRATRICOPEPTIDE REPEAT CONTAINING 2 | TMTC2 | −3.26585 | | −2.1169 |
| 8600 | TUMOR NECROSIS FACTOR (LIGAND) SUPERFAMILY, MEMBER 11 | TNFSF11 | −2.5807 | −5.88377 | |
| 23534 | TRANSPORTIN 3 | TNPO3 | −4.90495 | −4.06996 | −3.75153 |
| 10140 | TRANSDUCER OF ERBB2, 1 | TOB1 | −2.4024 | | −2.0188 |
| 9804 | TRANSLOCASE OF OUTER MITOCHONDRIAL MEMBRANE 20 HOMOLOG | TOMM20 | | −3.38974 | −2.88057 |
| | (YEAST) | | | | |
| 56993 | TRANSLOCASE OF OUTER MITOCHONDRIAL MEMBRANE 22 HOMOLOG | TOMM22 | | −2.1434 | −2.49763 |
| | (YEAST) | | | | |
| 58476 | TUMOR PROTEIN P53 INDUCIBLE NUCLEAR PROTEIN 2 | TP53INP2 | −2.66254 | −4.08177 | −2.05149 |
| 53373 | TWO PORE SEGMENT CHANNEL 1 | TPCN1 | −2.18053 | −2.14121 | −2.27751 |
| 7164 | TUMOR PROTEIN D52-LIKE 1 | TPD52L1 | −3.5544 | −3.25758 | |
| 7173 | THYROID PEROXIDASE | TPO | −2.12718 | −2.65698 | |
| 51693 | HEMATOPOIETIC STEM/PROGENITOR CELLS 176 | TRAPPC2L | −2.35046 | −2.84987 | |
| 54210 | TRIGGERING RECEPTOR EXPRESSED ON MYELOID CELLS 1 | TREM1 | −2.41212 | −4.62915 | −2.14621 |
| 55809 | TRANSCRIPTIONAL REGULATING FACTOR 1 | TRERF1 | −2.90946 | −4.16991 | |
| 8805 | TRIPARTITE MOTIF-CONTAINING 24 | TRIM24 | −1.97462 | −1.93153 | |
| 10155 | TRIPARTITE MOTIF-CONTAINING 28 | TRIM28 | | −2.13008 | −1.92375 |
| 166655 | TRIPARTITE MOTIF-CONTAINING 60 | TRIM60 | −1.98769 | −2.65534 | |
| 55128 | TRIPARTITE MOTIF-CONTAINING 68 | TRIM68 | −2.27145 | −3.97537 | |
| 6738 | TROVE DOMAIN FAMILY, MEMBER 2 | TROVE2 | −2.34569 | | −2.09889 |
| 7222 | TRANSIENT RECEPTOR POTENTIAL CATION CHANNEL, SUBFAMILY C, | TRPC3 | −2.25341 | | −2.81435 |
| | MEMBER 3 | | | | |
| 7225 | TRANSIENT RECEPTOR POTENTIAL CATION CHANNEL, SUBFAMILY C, | TRPC6 | −2.47758 | −2.44406 | −2.91983 |
| | MEMBER 6 | | | | |
| 140803 | HYPOTHETICAL PROTEIN FLJ20087 | TRPM6 | −3.44552 | −1.88594 | −2.78798 |
| 57616 | ZINC FINGER PROTEIN 537 | TSHZ3 | −1.90786 | −3.21475 | −2.10168 |
| 7259 | TSPY-LIKE 1 | TSPYL1 | −2.56945 | −3.16215 | −2.20417 |
| 7264 | TISSUE SPECIFIC TRANSPLANTATION ANTIGEN P35B | TSTA3 | −4.00686 | −2.37017 | |
| 26140 | TUBULIN TYROSINE LIGASE-LIKE FAMILY, MEMBER 3 | TTLL3 | −1.90887 | −1.88575 | |
| 84617 | TUBULIN, BETA 6 | TUBB6 | −2.01658 | −2.87146 | |
| 7284 | TU TRANSLATION ELONGATION FACTOR, MITOCHONDRIAL | TUFM | −2.92069 | −2.19069 | |
| 7294 | TXK TYROSINE KINASE | TXK | −2.78577 | −3.11976 | |
| 200081 | TAXILIN ALPHA | TXLNA | | −2.47492 | −2.55174 |
| 54957 | THIOREDOXIN-LIKE 4B | TXNL4B | −1.96591 | −2.3762 | −3.21574 |
| 7326 | UBIQUITIN-CONJUGATING ENZYME E2G 1 (UBC7 HOMOLOG, YEAST) | UBE2G1 | −1.86023 | −2.46231 | −2.00714 |
| 7326 | UBIQUITIN-CONJUGATING ENZYME E2G 1 (UBC7 HOMOLOG, YEAST) | UBE2G1 | −1.86023 | −2.46231 | −2.00714 |
| 9246 | UBIQUITIN-CONJUGATING ENZYME E2L 6 | UBE2L6 | −4.53705 | −3.29012 | |
| 143630 | HYPOTHETICAL PROTEIN MGC20470 | UBQLNL | | −1.96977 | −3.45196 |
| 92181 | DENDRITIC CELL-DERIVED UBIQUITIN-LIKE PROTEIN | UBTD2 | | −3.05707 | −1.8902 |
| 7993 | UBX DOMAIN CONTAINING 6 | UBXD6 | −2.07223 | −2.28693 | |
| 51569 | UBIQUITIN-FOLD MODIFIER 1 | UFM1 | | −2.44738 | −2.89156 |
| 167127 | UDP GLYCOSYLTRANSFERASE 3 FAMILY, POLYPEPTIDE A2 | UGT3A2 | −2.29205 | −1.92947 | |
| 121665 | DKFZP586C1324 PROTEIN | UNQ1887 | | −2.58128 | −2.02877 |
| 7398 | UBIQUITIN SPECIFIC PEPTIDASE 1 | USP1 | −1.91599 | −2.7275 | −2.81914 |
| 11274 | UBIQUITIN SPECIFIC PEPTIDASE 18 | USP18 | −3.79525 | −3.50393 | −3.57991 |
| 9960 | UBIQUITIN SPECIFIC PEPTIDASE 3 | USP3 | −3.70597 | | −2.06201 |
| 9098 | HYPERPOLYMORPHIC GENE 1 | USP6 | −1.85927 | −1.99963 | |
| 7405 | UV RADIATION RESISTANCE ASSOCIATED GENE | UVRAG | −3.54039 | −4.113 | |
| 8673 | VESICLE-ASSOCIATED MEMBRANE PROTEIN 8 (ENDOBREVIN) | VAMP8 | −1.86941 | | −2.06279 |
| 50853 | VILLIN-LIKE | VILL | −2.94995 | | −1.92166 |
| 7433 | VASOACTIVE INTESTINAL PEPTIDE RECEPTOR 1 | VIPR1 | −5.80786 | −4.69544 | −3.96241 |
| 79720 | VACUOLAR PROTEIN SORTING 37B (YEAST) | VPS37B | −1.85816 | −2.02574 | |
| 55275 | VACUOLAR PROTEIN SORTING 53 (YEAST) | VPS53 | −3.0736 | | −3.25681 |
| 128434 | CHROMOSOME 20 OPEN READING FRAME 102 | VSTM2L | −2.56128 | | −2.36445 |
| 340706 | VON WILLEBRAND FACTOR A DOMAIN CONTAINING 2 | VWA2 | −2.6436 | −1.88033 | |
| 11193 | WW DOMAIN BINDING PROTEIN 4 (FORMIN BINDING PROTEIN 21) | WBP4 | −2.88433 | | −1.89074 |
| 55759 | WD REPEAT DOMAIN 12 | WDR12 | −2.06357 | −2.34823 | |
| 79269 | WD REPEAT DOMAIN 32 | WDR32 | −2.20716 | −2.26755 | |
| 55339 | WD REPEAT DOMAIN 33 | WDR33 | −2.97558 | | −1.90321 |
| 401551 | SIMILAR TO HYPOTHETICAL PROTEIN FLJ25955 | WDR38 | −1.87216 | −2.76576 | −3.11598 |
| 139170 | WD REPEAT DOMAIN 40B | WDR40B | −2.30236 | | −2.23209 |
| 50717 | WD REPEAT DOMAIN 42A | WDR42A | −2.37447 | −2.61814 | −2.94676 |
| 348793 | WD REPEAT DOMAIN 53 | WDR53 | −4.71096 | −4.37789 | |
| 55100 | WD REPEAT DOMAIN 70 | WDR70 | −2.00896 | | −2.72161 |
| 79819 | WD REPEAT DOMAIN 78 | WDR78 | −3.82026 | −4.04859 | |
| 23038 | WD AND TETRATRICOPEPTIDE REPEATS 1 | WDTC1 | −2.08283 | −2.63992 | −2.30554 |
| 147179 | WIRE PROTEIN | WIPF2 | −2.63385 | −2.09502 | |
| 80014 | WW, C2 AND COILED-COIL DOMAIN CONTAINING 2 | WWC2 | −5.43289 | | −2.11183 |
| 51741 | PUTATIVE OXIDOREDUCTASE | WWOX | −2.98849 | | −2.36351 |
| 2829 | CHEMOKINE (C MOTIF) RECEPTOR 1 | XCR1 | −2.09586 | −2.71649 | |
| 286046 | CHROMOSOME 8 OPEN READING FRAME 7 | XKR6 | −2.09863 | −1.89435 | |
| 91419 | XRCC6 BINDING PROTEIN 1 | XRCC6BP1 | −2.27418 | −1.92721 | |
| 541465 | CANCER/TESTIS ANTIGEN CT45-2 | XX- | −2.78451 | −2.59265 | −2.14218 |
| | | FW88277B6.1 | | | |
| 10652 | SNARE PROTEIN YKT6 | YKT6 | −2.29434 | | −2.30459 |
| 79693 | YRDC DOMAIN CONTAINING (E. COLI) | YRDC | −3.01605 | −2.64556 | |
| 253943 | YTH DOMAIN FAMILY, MEMBER 3 | YTHDF3 | −2.76914 | −2.84077 | |
| 7532 | TYROSINE 3-MONOOXYGENASE/TRYPTOPHAN 5-MONOOXYGENASE | YWHAG | −3.31557 | −2.31734 | |
| | ACTIVATION PROTEIN, GAMMA POLYPEPTIDE | | | | |
| 7528 | YY1 TRANSCRIPTION FACTOR | YY1 | −3.29105 | −3.64289 | |
| 57684 | ZINC FINGER AND BTB DOMAIN CONTAINING 26 | ZBTB26 | −3.34289 | −3.49875 | |
| 9877 | ZINC FINGER CCCH-TYPE CONTAINING 11A | ZC3H11A | −2.84078 | | −3.43356 |
| 84240 | ZINC FINGER, CCHC DOMAIN CONTAINING 9 | ZCCHC9 | −3.2696 | −2.5597 | |
| 84936 | ZINC FINGER, FYVE DOMAIN CONTAINING 19 | ZFYVE19 | −2.05237 | −1.99609 | |
| 84217 | ZINC FINGER, MYND-TYPE CONTAINING 12 | ZMYND12 | −2.94854 | −3.47689 | −2.51531 |
| 118490 | ZINC FINGER, MYND-TYPE CONTAINING 17 | ZMYND17 | | −2.47856 | −1.89741 |
| 7690 | ZINC FINGER PROTEIN 131 (CLONE PHZ-10) | ZNF131 | −1.88418 | −2.64243 | −2.1069 |
| 7766 | ZINC FINGER PROTEIN 223 | ZNF223 | −1.97433 | −2.11869 | |
| 7572 | ZINC FINGER PROTEIN 24 (KOX 17) | ZNF24 | −2.06908 | −2.42245 | |
| 10224 | ZINC FINGER PROTEIN 443 | ZNF443 | −2.11094 | −2.36031 | |
| 114821 | ZINC FINGER PROTEIN 452 | ZNF452 | −3.09253 | −3.53396 | −2.54663 |
| 284443 | ZINC FINGER PROTEIN 493 | ZNF493 | −2.27517 | | −2.61513 |
| 22869 | ZINC FINGER PROTEIN 510 | ZNF510 | −5.67033 | −2.21998 | −2.82731 |
| 25925 | ZINC FINGER PROTEIN 521 | ZNF521 | −2.85699 | −2.52397 | |
| 147741 | ZINC FINGER PROTEIN 560 | ZNF560 | −2.22818 | −2.81243 | −2.94421 |
| 284346 | ZINC FINGER PROTEIN 575 | ZNF575 | −2.32433 | −2.1127 | −2.03963 |
| 169270 | ZINC FINGER PROTEIN 596 | ZNF596 | | −2.20356 | −2.17493 |
| 121274 | ZINC FINGER PROTEIN 641 | ZNF641 | −1.89321 | −2.1958 | |
| 146542 | ZINC FINGER PROTEIN 688 | ZNF688 | | −3.74763 | −2.11944 |
| 163051 | ZINC FINGER PROTEIN 709 | ZNF709 | −2.23282 | | −1.86393 |
| 7627 | ZINC FINGER PROTEIN 75A | ZNF75A | −3.62969 | −4.07762 | |
| 7629 | ZINC FINGER PROTEIN 76 (EXPRESSED IN TESTIS) | ZNF76 | −2.05861 | −2.09435 | |
| 7633 | ZINC FINGER PROTEIN 79 (PT7) | ZNF79 | −3.36041 | −2.94994 | |
| 30834 | ZINC RIBBON DOMAIN CONTAINING, 1 | ZNRD1 | −2.01992 | −3.05193 | |
| 23140 | ZINC FINGER, ZZ-TYPE WITH EF-HAND DOMAIN 1 | ZZEF1 | | −1.90416 | −2.10696 |

One or more currently preferred embodiments have been described by way of example. It will be apparent to persons skilled in the art that a number of variations and modifications can be made without departing from the scope of the invention as defined in the claims.

What is claimed is:

1. A method of reducing viability of a tumor cell in a subject, comprising the steps of:
introducing into a tumor cell in said subject an agent effective to modulate endoplasmic reticulum (ER) stress response and sensitize the tumour cell to cytolytic activity of an oncolytic virus in said subject; and
contacting the tumor cell with an oncolytic virus in an amount effective to reduce viability of the sensitized tumour cell,
wherein viability of the tumor cell is reduced.

2. The method of claim 1, wherein the agent is effective to enhance, diminish or inhibit the ER stress response in said subject.

3. (canceled)

4. The method of claim 1, wherein the agent is a siRNA specific to ERN, ATF6, Derlin1, Derlin2 or SEC61, a molecule effective to modulate ERN, ATF6, Derlin1, Derlin2 or SEC61 signaling, or a modified oncolytic virus wherein said modification renders the oncolytic virus effective to modulate ER stress response and sensitize the tumour cell to cytolytic activity.

5. (canceled)

6. The method of claim 1, wherein the tumour cell or cancer cell is selected from colon cancer, lung cancer, liver cancer, prostate cancer, bladder cancer, neck and mouth cancer, breast cancer, glioblastoma, lymphoma, carcinoma, renal cell cancer, pancreatic cancer, and ovarian cancer cells.

7. The method of claim 1, wherein the oncolytic virus is a native or modified herpes virus, Adenovirus, Adeno-associated virus, influenza virus, reovirus, rhabdovirus, Newcastle virus, vaccinia virus, poliovirus, measles virus, mumps virus, sindbis virus (SIN) or sendai virus (SV).

8. The method of claim 7, wherein the oncolytic virus is a native or modified rhabdovirus.

9. The method of claim 8, wherein the oncolytic virus is a native or modified vesicular stomatitis virus (VSV) or Maraba virus.

10. The method of claim 9, wherein the virus is a mutant virus modified with a function-improving mutation to make the virus a more effective cancer or tumour cell lysing agent.

11. A method of modulating sensitivity of cancer cells to infection by an oncolytic virus, the method comprising introducing into a cancer cell an agent effective to modulate endoplasmic reticulum (ER) stress response and sensitize the cancer cell to cytolytic activity of the oncolytic virus, wherein the cancer cells are sensitized to infection by the oncolytic virus.

12. A method of identifying a tumour cell sensitizing agent effective for sensitizing tumour cells to infection by an oncolytic virus, comprising:
providing a test molecule with putative endoplasmic reticulum (ER) stress response modulating activity,
adding the test molecule to a sample of said tumor cells,
contacting the tumor cells with the oncolytic virus, and
comparing cytolytic activity of the oncolytic virus in the sample of tumour cells with the test molecule to activity of the oncolytic virus in a sample of tumour cells without the test molecule,
wherein increased cytolytic activity of the oncolytic virus in the sample of tumour cells with the test molecule indicates the presence of a tumour cell sensitizing agent.

13. A compound effective to modulate endoplasmic reticulum (ER) stress response and sensitize a tumour cell to cytolytic activity of an oncolytic virus in a subject.

14. The compound of claim 13, wherein the compound is effective to inhibit the ER stress response in said subject.

15. The compound of claim 13, wherein the compound is a siRNA specific to ERN, ATF6, Derlin1, Derlin2 or SEC61 or a molecule effective to modulate ERN, ATF6, Derlin1, Derlin2 or SEC61 signaling.

16. The compound of claim 15, wherein the compound is effective to block or enhance ERN, ATF6, Derlin1, Derlin2 or SEC61 signaling.

17. The compound of claim 16, wherein the compound is a protein, a small molecule, a nucleic acid, or an antibody.

18. A composition comprising the compound of claim 13 and an acceptable carrier or excipient.

19. (canceled)

20. (canceled)

21. (canceled)

22. (canceled)

23. (canceled)

24. (canceled)

25. (canceled)

26. (canceled)

27. A method of sensitizing a tumor to cytolytic activity of an oncolytic virus, said method comprising inducing in a subject a mild stress to the endoplasmic reticulum (ER).

28. The method of claim 27, wherein inducing the mild stress comprises genetically disrupting an ER stress response gene.

29. The method of claim 28, wherein the ER stress response gene is selected from the group consisting of IRE1/ERN, DERLIN, and ATF6.

30. The method of claim 27, wherein inducing the mild stress comprises chemically inhibiting IRE1/ERN1

31. The method of claim 30, wherein compound 2 is administered to the subject to chemically inhibit IRE1/ERN1.

32. (canceled)

33. (canceled)

34. (canceled)

35. (canceled)

36. (canceled)

37. (canceled)

38. (canceled)

39. (canceled)
